SEQUENCE LISTING (27 sequences)

SEQ ID NO: 1 - protein, 406 aa, Synthetic construct
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 401 aa, synthetic construct
NVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSMEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWGGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVC

SEQ ID NO: 3 - protein, 925 aa, synthetic construct
MIHTNLKKKFSCCVLVFLLFAVICVWKEKKKGSYYDSFKLQTKEFQVLKSLGKLAMGSDSQSVSSSSTQDPHRGRQTLGSLRGLAKAKPGGGGSGGGGSGGGGSNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSMEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWGGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCGSGATNFSLLKQAGDVEENPGPMRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 4 - protein, 934 aa, synthetic construct
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGGGGGSGGGGSGGGGSNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSMEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWGGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCGSGATNFSLLKQAGDVEENPGPMRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 5 - protein, 398 aa, synthetic construct
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 6 - protein, 22 aa, Synthetic construct
GSGATNFSLLKQAGDVEENPGP

SEQ ID NO: 7 - protein, 21 aa, synthetic construct
GSGEGRGSLLTCGDVEENPGP

SEQ ID NO: 8 - protein, 23 aa, synthetic construct
GSGQCTNYALLKLAGDVESNPGP

SEQ ID NO: 9 - protein, 25 aa, synthetic construct
GSGVKQTLNFDLLKLAGDVESNPGP

SEQ ID NO: 10 - DNA, 1221 bp, synthetic construct
  source 1..1221: mol_type = other DNA
atgatccacaccaatctgaagaagaagtttagctgttgcgtgctggtgttcctgctgttcgctgtgatctgcgtgtggaaggagaagaagaagggcagctattatgactcctttaagctgcagaccaaggagttccaggtgctgaagtccctgggcaagctggccatgggcagcgactcccagtccgtgagctcctccagcacccaggacccccaccggggaaggcagaccctgggatccctgaggggcctggctaaggccaagcctgaggcttccttccaggtgtggaataaggatagctccagcaagaacctgatccctaggctgcagaagatctggaagaactatctgagcatgaacaagtataaggtgagctataagggccctggccctggcatcaagttctccgccgaggccctgaggtgccacctgagagatcacgtgaatgtgagcatggtggaggtgaccgacttcccttttaacacctccgagtgggagggctacctgcccaaggagagcatccggaccaaggccggcccttggggcagatgtgccgtggtgtccagcgccggctccctgaagagctcccagctgggccgggagatcgatgaccacgatgctgtgctgaggttcaatggcgctcctaccgctaactttcagcaggatgtgggcaccaagaccaccatccggctgatgaatagccagctggtgaccaccgagaagcggtttctgaaggactccctgtacaacgagggcatcctgatcgtgtgggacccctccgtgtaccacagcgacatccccaagtggtatcagaaccccgactacaacttcttcaataattacaagacctaccggaagctgcaccccaaccagcccttttatatcctgaagccccagatgccttgggagctgtgggacatcctgcaggagatctcccccgaggagatccagcctaacccccctagcagcggcatgctgggcatcatcatcatgatgaccctgtgcgatcaggtggatatctacgagttcctgcccagcaagcggaagaccgacgtgtgttattattatcagaagttcttcgattccgcctgcaccatgggcgcctatcaccctctgctgtacgagaagaatctggtgaagcacctgaaccagggcaccgacgaggacatctatctgctgggcaaggccaccctgcccggctttaggaccatccactgctga

SEQ ID NO: 11 - DNA, 1545 bp, synthetic construct
  source 1..1545: mol_type = other DNA
atgaaaaactttttattattaactttaatattacttactgcttgtaataattcagaagaaaatacacaatctattattaaaaatgatattaataaaactattattgatgaggagtatgttaatttagagccaattaatcaatcaaacatctcttttacaaaacactcttgggtacaaacttgtggtacgcaacaactattaacagaacaaaataaagagtcaatatcattatctgtagtggcgccacgattagatgacgatgaaaagtactgctttgattttaatggtgttagtaataaaggtgaaaaatatataacaaaagtaacattaaacgtagtggctccatctttagaggtttatgttgatcatgcatctcttccaactcttcagcagctaatggatattattaaatcggaagaagaaaatcctacagcacaaagatatatagcttgggggagaatagttccgactgatgagcaaatgaaagagttaaatattacatcgtttgcattgataaataaccatacaccagctgacttagtacaagaaattgttaagcaagcacaaacaaagcatagattgaatgttaaacttagctctaacactgctcattcatttgataatttagtgccaatactaaaagaattaaattcgtttaataacgttacggtaacaaatatagatttatatgatgatggttcagcagaatatgtaaatttatataactggagagatacattaaataaaacagataatttaaaaattggtaaagattatcttgaggatgtcattaatggtatcaatgaagacacttcaaatacaggaacatcatctgtttataactggcaaaaactatatccagctaactaccattttttaagaaaagattatttaactttagaaccatcattacatgagttacgagactatattggtgatagtttaaagcaaatgcaatgggatggtttcaaaaaattcaatagcaaacaacaagaattattcttatcgattgttaattttgacaaacaaaaattacaaaatgaatataattcatctaatttaccaaactttgtgtttacaggtacgactgtatgggctggtaaccatgaaagagagtattatgcgaaacaacaaattaatgtcattaataatgcaattaatgaatcgagcccacattatttaggcaatagttatgatttgttcttcaaaggtcaccctggtggcggtatcattaatacattaataatgcaaaactatccttcaatggttgatattccatcaaaaatatcatttgaagttttgatgatgacagatatgcttcctgatgcagttgctggtatagcgagctctttatatttcacgataccagctgaaaaaattaaatttatagtttttacatcgacagaaactataactgatcgtgaaactgctttgagaagtcctttagttcaagtaatgataaaactaggtattgtaaaagaagagaatgtacttttttgggctgatctgccaaattgtgaaacaggtgtttgtattgcagtctag

SEQ ID NO: 12 - DNA, 2778 bp, synthetic construct
  source 1..2778: mol_type = other DNA
atgatccacaccaacctgaagaagaagttctcctgctgtgtgctggtgtttctgctgttcgctgtgatctgcgtgtggaaggagaagaagaagggcagctattatgacagctttaagctgcagaccaaggagtttcaggtgctgaagtccctgggcaagctggccatgggctccgactcccagtccgtgtccagctccagcacccaggatcctcaccggggcaggcagaccctgggctctctgagaggcctggctaaggctaagcccggcggcggaggcagcggaggaggaggatctggcggcggaggatccaatgtggtggccccttccctggaggtgtatgtggaccacgcctccctgcccaccctgcagcagctgatggatatcatcaagagcgaggaggagaatcctaccgcccagcggtatatcgcctggggccggatcgtgcctaccgacgagcagatgaaggagctgaacatcacctccttcgctctgatcaacaatcacacccccgccgacctggtgcaggagatcgtgaagcaggcccagaccaagcaccggctgaacgtgaagctgtcctccaacaccgcccactcctttgacaacctggtgcctatcctgaaggagctgaattcctttaataacgtgaccgtgaccaacatcgacctgtacgacgacggctccatggagtatgtgaacctgtataactggagggacaccctgaataagaccgacaatctgaagatcggcaaggactatctggaggacgtgatcaatggcatcaatgaggacacctccaacaccggcaccagcagcgtgtataactggcagaagctgtaccctgctaattaccactttctgaggaaggattatctgaccctggagccttccctgcacgagctgagggactacatcggcgatagcctgaagcagatgcagtgggatggctttaagaagttcaacagcaagcagcaggagctgtttctgagcatcgtgaattttgacaagcagaagctgcagaacgagtataactcctccaatctgcctaactttgtgttcaccggcaccaccgtgtggggcggcaatcacgagcgggagtactatgccaagcagcagatcaacgtgatcaataatgccatcaatgagagcagcccccactacctgggcaatagctacgatctgttcttcaagggccaccctggcggcggcatcatcaacaccctgatcatgcagaactatccttccatggtggatatccctagcaagatctccttcgaggtgctgatgatgaccgacatgctgcccgacgccgtggctggcatcgcctctagcctgtattttaccatccccgctgagaagatcaagttcatcgtgtttaccagcaccgagaccatcaccgaccgggagaccgccctgaggtccccactggtgcaggtgatgatcaagctgggcatcgtgaaggaggagaacgtgctgttttgggccgacctgcccaactgtgagaccggcgtgtgtggctccggcgctaccaacttctccctgctgaagcaggctggcgatgtggaggagaatcccggccctatgcggctgcgggagcccctgttgtccggctctgccgctatgcccggcgcttccctgcagagagcctgtcggctgctggtggctgtgtgcgctctgcacctgggcgtgaccctggtgtactatctggctggccgggatctgagccggctgcctcagctggtgggcgtgagcacccccctgcagggaggatccaactccgccgctgccatcggccagtcctccggagagctgcggaccggaggcgctaggcctccaccaccactgggcgcttcttcccagcctcggcccggaggagatagcagccccgtggtggactccggccctggacctgcttccaacctgaccagcgtgcccgtgcctcacaccaccgctctgtccctgcccgcctgtcccgaggagtcccctctgctggtgggccctatgctgatcgagtttaatatgcctgtggacctggagctggtggctaagcagaatcctaacgtgaagatgggcggcaggtacgctcccagggactgcgtgagccctcacaaggtggccatcatcatccctttccggaaccggcaggagcacctgaagtactggctgtactacctgcaccctgtgctgcagaggcagcagctggactatggcatctacgtgatcaaccaggccggcgacaccatctttaaccgggccaagctgctgaatgtgggctttcaggaggccctgaaggattatgactacacctgctttgtgtttagcgatgtggatctgatccctatgaacgatcacaacgcctaccggtgcttcagccagcctcggcacatcagcgtggccatggacaagttcggcttctccctgccttacgtgcagtacttcggcggcgtgtccgccctgtccaagcagcagttcctgaccatcaacggctttcctaataactactggggctggggcggcgaggatgatgacatctttaatcggctggtgtttaggggcatgagcatcagccggcctaacgccgtggtgggcaggtgcaggatgatcaggcactcccgggacaagaagaacgagcctaatcctcagaggtttgacaggatcgctcacaccaaggagaccatgctgagcgacggcctgaatagcctgacctaccaggtgctggacgtgcagaggtaccctctgtatacccagatcaccgtggatatcggcacccctagctga

SEQ ID NO: 13 - DNA, 1431 bp, synthetic construct
  source 1..1431: mol_type = other DNA
atgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggcggaggcagcggaggaggaggatctggcggcggaggatccaatgtggtggccccttccctggaggtgtatgtggaccacgcctccctgcccaccctgcagcagctgatggatatcatcaagagcgaggaggagaatcctaccgcccagcggtatatcgcctggggccggatcgtgcctaccgacgagcagatgaaggagctgaacatcacctccttcgctctgatcaacaatcacacccccgccgacctggtgcaggagatcgtgaagcaggcccagaccaagcaccggctgaacgtgaagctgtcctccaacaccgcccactcctttgacaacctggtgcctatcctgaaggagctgaattcctttaataacgtgaccgtgaccaacatcgacctgtacgacgacggctccatggagtatgtgaacctgtataactggagggacaccctgaataagaccgacaatctgaagatcggcaaggactatctggaggacgtgatcaatggcatcaatgaggacacctccaacaccggcaccagcagcgtgtataactggcagaagctgtaccctgctaattaccactttctgaggaaggattatctgaccctggagccttccctgcacgagctgagggactacatcggcgatagcctgaagcagatgcagtgggatggctttaagaagttcaacagcaagcagcaggagctgtttctgagcatcgtgaattttgacaagcagaagctgcagaacgagtataactcctccaatctgcctaactttgtgttcaccggcaccaccgtgtggggcggcaatcacgagcgggagtactatgccaagcagcagatcaacgtgatcaataatgccatcaatgagagcagcccccactacctgggcaatagctacgatctgttcttcaagggccaccctggcggcggcatcatcaacaccctgatcatgcagaactatccttccatggtggatatccctagcaagatctccttcgaggtgctgatgatgaccgacatgctgcccgacgccgtggctggcatcgcctctagcctgtattttaccatccccgctgagaagatcaagttcatcgtgtttaccagcaccgagaccatcaccgaccgggagaccgccctgaggtccccactggtgcaggtgatgatcaagctgggcatcgtgaaggaggagaacgtgctgttttgggccgacctgcccaactgtgagaccggcgtgtgt

SEQ ID NO: 14 - DNA, 1197 bp, synthetic construct
  source 1..1197: mol_type = other DNA
atgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggaagcaatagcgccgctgccatcggccagtcctccggagagctgcggaccggaggcgctaggccacctccacctctgggcgcttcctcccagcctcggcctggaggagatagcagccccgtggtggatagcggccctggcccagcttctaacctgaccagcgtgcctgtgccccacaccaccgccctgagcctgcctgcttgccccgaggagtcccccctgctggtgggaccaatgctgatcgagttcaatatgcctgtggatctggagctggtggccaagcagaatcccaatgtgaagatgggcggccggtacgctccccgggattgtgtgtcccctcacaaggtggctatcatcatccctttccggaaccggcaggagcacctgaagtactggctgtattacctgcaccccgtgctgcagaggcagcagctggactatggcatctacgtgatcaatcaggctggcgacaccatcttcaatcgggctaagctgctgaacgtgggctttcaggaggctctgaaggactacgactacacctgctttgtgttcagcgatgtggacctgatccccatgaacgaccacaacgcttataggtgcttctcccagcctaggcacatctccgtggctatggacaagtttggcttctccctgccctacgtgcagtattttggcggcgtgtccgccctgagcaagcagcagtttctgaccatcaatggctttcctaacaattactggggctggggcggcgaggatgatgacatcttcaaccggctggtgttcaggggcatgtccatcagcaggcctaacgctgtggtgggccggtgtaggatgatcaggcactcccgggacaagaagaatgagcccaatcctcagcggtttgaccggatcgcccacaccaaggagaccatgctgtccgacggcctgaattccctgacctaccaggtgctggacgtgcagaggtatcctctgtacacccagatcaccgtggatatcggcaccccttcctga

SEQ ID NO: 15 - protein, 313 aa, synthetic construct
MFTPVRRRVRTAALALSAAAALVLGSTAASGASATPSPAPAPAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 16 - protein, 843 aa, synthetic construct
MDKHLLVKRTLGCVCAATLMGAALATHHDSLNTVKAEEKTVQTGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

SEQ ID NO: 17 - DNA, 1104 bp, synthetic construct
  source 1..1104: mol_type = other DNA
gactcgaagcactcgctcggcgcttccgactggcggggcgccgaccatcccgttgggagaccatcagccatgttcactccggttcgcagaagggtgcggacggctgcgctcgcgctctcggccgccgcggccctcgtcctcggttccaccgccgcgagcggcgcgtcagcgaccccctcacccgctccggccccggccccggccccggtgaagcaggggccgacctcggtggcctacgtcgaggtgaacaacaacagcatgctcaacgtcggcaagtacaccctggcggacggaggcggcaacgccttcgacgtagccgtgatcttcgcggcgaacatcaactacgacaccggcacgaagacggcctacctgcacttcaacgagaacgtgcagcgcgtccttgacaacgctgtcacgcagatacggccgttgcagcaacagggcatcaaggtcctcctctcggtgctcggcaaccaccagggcgccgggttcgcgaacttcccctcacagcaggcggcttcggcgttcgcgaagcagctctcggacgccgtggcgaagtacggcctcgacggcgtcgacttcgacgacgaatacgccgagtacggcaacaacggcaccgcgcagcccaacgacagttcgttcgtgcacctggtgacggcactgcgcgcgaacatgcccgacaagatcatcagcctctacaacatcggcccggccgcgtcccgcctgtcgtacggcggtgtcgacgtctccgacaagttcgactacgcctggaatccctactacggcacctggcaggtccccggcatcgcactgcccaaggcgcagctgtcgccggcggccgtcgagatcggccggacctcacggagcaccgtcgccgacctcgcccgtcgcaccgtcgacgaggggtacggcgtctatctgacgtacaacctcgacggcggcgatcgcaccgccgacgtctccgcgttcaccagggagctgtacggcagcgaggcggtccggacgccgtaggggcgtcggggcctgccgtcagtccagtacgaaggtgccgccggcggtggtcgcctggccgtgcccgaaagcggccgccggcgtccaggatcc

SEQ ID NO: 18 - DNA, 2776 bp, synthetic construct
  source 1..2776: mol_type = other DNA
ggcgaactataggaatgcgcttacatagatggtatatcagatgggaagccatgacttagtaccaaaaataaggagtgtccaaatggataaacatttgttggtaaaaagaacactagggtgtgtttgtgctgcaacgttgatgggagctgccttagcgacccaccatgattcactcaatactgtaaaagcggaggagaagactgttcaaacaggaaagacagatcagcaggttggtgctaaattggtacaggaaatccgtgaaggaaaacgcggaccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatgaccgaagacaaacgctatgatgtcattgatcaaaaagacattcctgacccagcattaagagaacaaatcattcaacaagttggacagtataaaggcgatttggaacgttataacaagacattggtgcttacaggagataagattcaaaatcttaaaggactagaaaaattaagcaagttacaaaaattagagttgcgccagctatctaacgttaaagaaattactccagaacttttgccggaaagcatgaaaaaagatgctgagcttgttatggtaggcatgactggtttagaaaaactaaaccttagtggtctaaatcgtcaaactttagacggtatagacgtgaatagtattacgcatttgacatcatttgatatttcacataatagtttggacttgtcggaaaagagtgaagaccgtaaactattaatgactttgatggagcaggtttcaaatcatcaaaaaataacggtgaaaaatacggcttttgaaaatcaaaaaccgaaaggttattatcctcagacgtatgataccaaagaaggtcattatgatgttgataatgcagaacatgatattttaactgattttgtttttggaactgttactaaacgtaatacctttattggagacgaagaagcatttgctatctataaagaaggagctgtcgatggtcgacaatatgtgtctaaagactatacttatgaagcttttcgtaaagactataaaggttacaaggttcatttaactgcttctaacctaggagaaacagttacttctaaggtaactgctactactgatgaaacttacttagtagatgtttctgatggggaaaaagttgttcaccacatgaaactcaatataggatctggtgccatcatgatggaaaatctggcaaaaggggctaaagtgattggtacatctggggactttgagcaagcaaagaagattttcgatggtgaaaagtcagatagattcttcacttggggacaaactaactggatagcttttgatctaggagaaattaatcttgcgaaggaatggcgtttatttaatgcagagacaaatactgaaataaagacagatagtagcttaaacgtggctaaaggacgtcttcagattttaaaagatacaactattgatttagaaaaaatggacataaaaaatcgtaaagagtatctgtcgaatgatgaaaattggactgatgttgctcagatggatgatgcaaaagcgatatttaatagtaaattatccaatgttttatctcggtattggcggttttgtgtagatggtggagctagctcttattaccctcaatataccgaacttcaaatcctcggacaacgtttatcaaatgatgtcgctaatacgctgaaggattgatattaaagtcctacgattacaaagataaacagtttctcaaagttgatttaagcactttaaaacctaactaaaaatctgagatgaatagtcccagatttttagtcttttataggttttgatgacataaagctaaataatcgttagactaccagaaagtggcgc

SEQ ID NO: 19 - DNA, 66 bp, synthetic construct
  source 1..66: mol_type = other DNA
ggctccggcgctaccaacttctccctgctgaagcaggctggcgatgtggaggagaatcccggccct

SEQ ID NO: 20 - DNA, 63 bp, synthetic construct
  source 1..63: mol_type = other DNA
ggttcaggtgagggccgcggcagtctgctgacttgcggcgacgtcgaggagaaccccggtcct

SEQ ID NO: 21 - DNA, 69 bp, synthetic construct
  source 1..69: mol_type = other DNA
gggagtgggcaatgcactaactatgcattgcttaagttggctggtgatgtagaatctaatcctgggccc

SEQ ID NO: 22 - DNA, 75 bp, synthetic construct
  source 1..75: mol_type = other DNA
ggcagtggggtgaagcagaccctcaattttgacttgttgaaactcgcaggcgacgtagaatctaaccccggacct

SEQ ID NO: 23 - DNA, 7840 bp, synthetic construct
  source 1..7840: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgatccacaccaatctgaagaagaagtttagctgttgcgtgctggtgttcctgctgttcgctgtgatctgcgtgtggaaggagaagaagaagggcagctattatgactcctttaagctgcagaccaaggagttccaggtgctgaagtccctgggcaagctggccatgggcagcgactcccagtccgtgagctcctccagcacccaggacccccaccggggaaggcagaccctgggatccctgaggggcctggctaaggccaagcctgaggcttccttccaggtgtggaataaggatagctccagcaagaacctgatccctaggctgcagaagatctggaagaactatctgagcatgaacaagtataaggtgagctataagggccctggccctggcatcaagttctccgccgaggccctgaggtgccacctgagagatcacgtgaatgtgagcatggtggaggtgaccgacttcccttttaacacctccgagtgggagggctacctgcccaaggagagcatccggaccaaggccggcccttggggcagatgtgccgtggtgtccagcgccggctccctgaagagctcccagctgggccgggagatcgatgaccacgatgctgtgctgaggttcaatggcgctcctaccgctaactttcagcaggatgtgggcaccaagaccaccatccggctgatgaatagccagctggtgaccaccgagaagcggtttctgaaggactccctgtacaacgagggcatcctgatcgtgtgggacccctccgtgtaccacagcgacatccccaagtggtatcagaaccccgactacaacttcttcaataattacaagacctaccggaagctgcaccccaaccagcccttttatatcctgaagccccagatgccttgggagctgtgggacatcctgcaggagatctcccccgaggagatccagcctaacccccctagcagcggcatgctgggcatcatcatcatgatgaccctgtgcgatcaggtggatatctacgagttcctgcccagcaagcggaagaccgacgtgtgttattattatcagaagttcttcgattccgcctgcaccatgggcgcctatcaccctctgctgtacgagaagaatctggtgaagcacctgaaccagggcaccgacgaggacatctatctgctgggcaaggccaccctgcccggctttaggaccatccactgcggcagcggcgctaccaatttcagcctgctgaagcaggctggcgatgtggaggagaaccccggccccatgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggaagcaatagcgccgctgccatcggccagtcctccggagagctgcggaccggaggcgctaggccacctccacctctgggcgcttcctcccagcctcggcctggaggagatagcagccccgtggtggatagcggccctggcccagcttctaacctgaccagcgtgcctgtgccccacaccaccgccctgagcctgcctgcttgccccgaggagtcccccctgctggtgggaccaatgctgatcgagttcaatatgcctgtggatctggagctggtggccaagcagaatcccaatgtgaagatgggcggccggtacgctccccgggattgtgtgtcccctcacaaggtggctatcatcatccctttccggaaccggcaggagcacctgaagtactggctgtattacctgcaccccgtgctgcagaggcagcagctggactatggcatctacgtgatcaatcaggctggcgacaccatcttcaatcgggctaagctgctgaacgtgggctttcaggaggctctgaaggactacgactacacctgctttgtgttcagcgatgtggacctgatccccatgaacgaccacaacgcttataggtgcttctcccagcctaggcacatctccgtggctatggacaagtttggcttctccctgccctacgtgcagtattttggcggcgtgtccgccctgagcaagcagcagtttctgaccatcaatggctttcctaacaattactggggctggggcggcgaggatgatgacatcttcaaccggctggtgttcaggggcatgtccatcagcaggcctaacgctgtggtgggccggtgtaggatgatcaggcactcccgggacaagaagaatgagcccaatcctcagcggtttgaccggatcgcccacaccaaggagaccatgctgtccgacggcctgaattccctgacctaccaggtgctggacgtgcagaggtatcctctgtacacccagatcaccgtggatatcggcaccccttcctgatgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 24 - DNA, 8131 bp, synthetic construct
  source 1..8131: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgatccacaccaacctgaagaagaagttctcctgctgtgtgctggtgtttctgctgttcgctgtgatctgcgtgtggaaggagaagaagaagggcagctattatgacagctttaagctgcagaccaaggagtttcaggtgctgaagtccctgggcaagctggccatgggctccgactcccagtccgtgtccagctccagcacccaggatcctcaccggggcaggcagaccctgggctctctgagaggcctggctaaggctaagcccggcggcggaggcagcggaggaggaggatctggcggcggaggatccaatgtggtggccccttccctggaggtgtatgtggaccacgcctccctgcccaccctgcagcagctgatggatatcatcaagagcgaggaggagaatcctaccgcccagcggtatatcgcctggggccggatcgtgcctaccgacgagcagatgaaggagctgaacatcacctccttcgctctgatcaacaatcacacccccgccgacctggtgcaggagatcgtgaagcaggcccagaccaagcaccggctgaacgtgaagctgtcctccaacaccgcccactcctttgacaacctggtgcctatcctgaaggagctgaattcctttaataacgtgaccgtgaccaacatcgacctgtacgacgacggctccatggagtatgtgaacctgtataactggagggacaccctgaataagaccgacaatctgaagatcggcaaggactatctggaggacgtgatcaatggcatcaatgaggacacctccaacaccggcaccagcagcgtgtataactggcagaagctgtaccctgctaattaccactttctgaggaaggattatctgaccctggagccttccctgcacgagctgagggactacatcggcgatagcctgaagcagatgcagtgggatggctttaagaagttcaacagcaagcagcaggagctgtttctgagcatcgtgaattttgacaagcagaagctgcagaacgagtataactcctccaatctgcctaactttgtgttcaccggcaccaccgtgtggggcggcaatcacgagcgggagtactatgccaagcagcagatcaacgtgatcaataatgccatcaatgagagcagcccccactacctgggcaatagctacgatctgttcttcaagggccaccctggcggcggcatcatcaacaccctgatcatgcagaactatccttccatggtggatatccctagcaagatctccttcgaggtgctgatgatgaccgacatgctgcccgacgccgtggctggcatcgcctctagcctgtattttaccatccccgctgagaagatcaagttcatcgtgtttaccagcaccgagaccatcaccgaccgggagaccgccctgaggtccccactggtgcaggtgatgatcaagctgggcatcgtgaaggaggagaacgtgctgttttgggccgacctgcccaactgtgagaccggcgtgtgtggctccggcgctaccaacttctccctgctgaagcaggctggcgatgtggaggagaatcccggccctatgcggctgcgggagcccctgttgtccggctctgccgctatgcccggcgcttccctgcagagagcctgtcggctgctggtggctgtgtgcgctctgcacctgggcgtgaccctggtgtactatctggctggccgggatctgagccggctgcctcagctggtgggcgtgagcacccccctgcagggaggatccaactccgccgctgccatcggccagtcctccggagagctgcggaccggaggcgctaggcctccaccaccactgggcgcttcttcccagcctcggcccggaggagatagcagccccgtggtggactccggccctggacctgcttccaacctgaccagcgtgcccgtgcctcacaccaccgctctgtccctgcccgcctgtcccgaggagtcccctctgctggtgggccctatgctgatcgagtttaatatgcctgtggacctggagctggtggctaagcagaatcctaacgtgaagatgggcggcaggtacgctcccagggactgcgtgagccctcacaaggtggccatcatcatccctttccggaaccggcaggagcacctgaagtactggctgtactacctgcaccctgtgctgcagaggcagcagctggactatggcatctacgtgatcaaccaggccggcgacaccatctttaaccgggccaagctgctgaatgtgggctttcaggaggccctgaaggattatgactacacctgctttgtgtttagcgatgtggatctgatccctatgaacgatcacaacgcctaccggtgcttcagccagcctcggcacatcagcgtggccatggacaagttcggcttctccctgccttacgtgcagtacttcggcggcgtgtccgccctgtccaagcagcagttcctgaccatcaacggctttcctaataactactggggctggggcggcgaggatgatgacatctttaatcggctggtgtttaggggcatgagcatcagccggcctaacgccgtggtgggcaggtgcaggatgatcaggcactcccgggacaagaagaacgagcctaatcctcagaggtttgacaggatcgctcacaccaaggagaccatgctgagcgacggcctgaatagcctgacctaccaggtgctggacgtgcagaggtaccctctgtatacccagatcaccgtggatatcggcacccctagctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 25 - DNA, 6573 bp, synthetic construct
  source 1..6573: mol_type = other DNA
gacggatcgggagatctcccgatcccctatggtgcactctcagtacaatctgctcgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgatccacaccaatctgaagaagaagtttagctgttgcgtgctggtgttcctgctgttcgctgtgatctgcgtgtggaaggagaagaagaagggcagctattatgactcctttaagctgcagaccaaggagttccaggtgctgaagtccctgggcaagctggccatgggcagcgactcccagtccgtgagctcctccagcacccaggacccccaccggggaaggcagaccctgggatccctgaggggcctggctaaggccaagcctgaggcttccttccaggtgtggaataaggatagctccagcaagaacctgatccctaggctgcagaagatctggaagaactatctgagcatgaacaagtataaggtgagctataagggccctggccctggcatcaagttctccgccgaggccctgaggtgccacctgagagatcacgtgaatgtgagcatggtggaggtgaccgacttcccttttaacacctccgagtgggagggctacctgcccaaggagagcatccggaccaaggccggcccttggggcagatgtgccgtggtgtccagcgccggctccctgaagagctcccagctgggccgggagatcgatgaccacgatgctgtgctgaggttcaatggcgctcctaccgctaactttcagcaggatgtgggcaccaagaccaccatccggctgatgaatagccagctggtgaccaccgagaagcggtttctgaaggactccctgtacaacgagggcatcctgatcgtgtgggacccctccgtgtaccacagcgacatccccaagtggtatcagaaccccgactacaacttcttcaataattacaagacctaccggaagctgcaccccaaccagcccttttatatcctgaagccccagatgccttgggagctgtgggacatcctgcaggagatctcccccgaggagatccagcctaacccccctagcagcggcatgctgggcatcatcatcatgatgaccctgtgcgatcaggtggatatctacgagttcctgcccagcaagcggaagaccgacgtgtgttattattatcagaagttcttcgattccgcctgcaccatgggcgcctatcaccctctgctgtacgagaagaatctggtgaagcacctgaaccagggcaccgacgaggacatctatctgctgggcaaggccaccctgcccggctttaggaccatccactgctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 26 - DNA, 6550 bp, synthetic construct
  source 1..6550: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggaagcaatagcgccgctgccatcggccagtcctccggagagctgcggaccggaggcgctaggccacctccacctctgggcgcttcctcccagcctcggcctggaggagatagcagccccgtggtggatagcggccctggcccagcttctaacctgaccagcgtgcctgtgccccacaccaccgccctgagcctgcctgcttgccccgaggagtcccccctgctggtgggaccaatgctgatcgagttcaatatgcctgtggatctggagctggtggccaagcagaatcccaatgtgaagatgggcggccggtacgctccccgggattgtgtgtcccctcacaaggtggctatcatcatccctttccggaaccggcaggagcacctgaagtactggctgtattacctgcaccccgtgctgcagaggcagcagctggactatggcatctacgtgatcaatcaggctggcgacaccatcttcaatcgggctaagctgctgaacgtgggctttcaggaggctctgaaggactacgactacacctgctttgtgttcagcgatgtggacctgatccccatgaacgaccacaacgcttataggtgcttctcccagcctaggcacatctccgtggctatggacaagtttggcttctccctgccctacgtgcagtattttggcggcgtgtccgccctgagcaagcagcagtttctgaccatcaatggctttcctaacaattactggggctggggcggcgaggatgatgacatcttcaaccggctggtgttcaggggcatgtccatcagcaggcctaacgctgtggtgggccggtgtaggatgatcaggcactcccgggacaagaagaatgagcccaatcctcagcggtttgaccggatcgcccacaccaaggagaccatgctgtccgacggcctgaattccctgacctaccaggtgctggacgtgcagaggtatcctctgtacacccagatcaccgtggatatcggcaccccttcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 27 - DNA, 8201 bp, synthetic construct
  source 1..8201: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctaagcttgccgccaccatgagggtccccgctcagctcctggggctcctgctgctctggctcccaggtgcacgatgtgatatccaaatgactcaaagtccaagtagtctgtccgcaagcgtcggcgatcgcgtgaccatcacatgtagagcttctcaaggcatccggaactacctggcttggtaccagcagaagcctggcaaggcccccaagctgctgatctatgccgcttctaccctccagtccggcgtgcctagcagattttccggctccggatctggaacagacttcaccctgaccatctcctctctgcagcctgaggacgtggccacctactactgccagcggtacaacagagccccatacaccttcggccagggcaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgaggatccactagtccagtgtggtggaattcagatccgttaacggttaccaactacctagactggattcgtgacaacatgcggccgtgatatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacagctatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgggtacgtcctcacattcagtgatcagcactgaacacagacccgcggccgctcgaggccgccaccatgggttggagcctcatcttgctcttccttgtcgctgttgctacgcgtgtcgaagtgcaactagtggaaagtggtggtggtctcgtgcagcccggaaggtctctgcggctgtcctgtgctgcttccggcttcaccttcgacgattatgccatgcactgggtccggcaagctcctggcaagggcctggagtgggtctctgccatcacctggaattctggccacatcgactacgccgactccgtggaaggcagatttaccatctccagagacaacgccaagaacagcctgtacctgcagatgaactccctgagagccgaggatacagccgtgtactactgcgccaaagtgtcctacctgtctaccgcttctagcctggactactggggccagggcaccctggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2025/0333690 A1
(43) Pub. Date: Oct. 30, 2025

(54) CELLS FOR GLYCOENGINEERING AND METHODS OF USE

(71) Applicant: Rock BioMedical Inc., Taipei City (TW)

(72) Inventors: Chi-Huey WONG, Rancho Santa Fe, CA (US); Szu-Wen WANG, Taipei City (TW); Jeng Shin LEE, Lincoln, MA (US)

(21) Appl. No.: 18/747,694

(22) Filed: Jun. 19, 2024

Related U.S. Application Data
(60) Provisional application No. 63/581,389, filed on Sep. 8, 2023.
(60) Provisional application No. 63/639,286, filed on Apr. 26, 2024.

(51) Int. Cl. C12N 5/073; C12N 9/10; C12P 21/00

(52) U.S. Cl. CPC C12N 5/0604; C12N 9/1051; C12N 9/1081; C12P 21/005; C12N 2510/00; C12Y 204/01038; C12Y 204/99001

(57) ABSTRACT

The present disclosure relates to glycoengineering, including cells and methods for glycoengineering a recombinant glycoprotein, whereby the produced glycoproteins are conjugated with desired glycans.

### CROSS-REFERENCE TO RELATED APPLICATIONS

This application claims the priority of U.S. Provisional Patent Applications No. 63/639,286, filed on Apr. 26, 2024. The entirety of the aforementioned application is incorporated herein by reference.

### SEQUENCE LISTING

The instant application contains a Sequence Listing which is submitted electronically in .xml format and is hereby incorporated by reference in its entirety. The .xml copy, created on Jun. 17, 2024, is named “A1000-01700US_20240617_SeqListing.xml” and is 78,281 bytes in size.

### FIELD

The present disclosure is related to compositions and methods for glycoprotein engineering and production of glycoengineered therapeutic antibodies.

### BACKGROUND OF THE INVENTION

Over the past few years, protein therapeutics have gained increasing prominence in almost every field of medicine. Most of these protein therapeutics are glycoproteins, and the glycans on those glycoproteins have been proven to be essential and determinative of therapeutic efficacy. Therefore, post-translational modifications of these biologics, particularly towards their glycosylation states, have drawn increased attention in academic and industrial sectors. With the ongoing trend to produce protein therapeutics in mammalian expression platforms, there is a continuing need for platforms that offer a desired glycan profile that is favorable for improved efficacy.

### BRIEF SUMMARY OF THE INVENTION

In one aspect, the present disclosure provides a cell for expressing a sialylated glycoprotein wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity.

In one aspect, the present disclosure provides a method for glycoengineering a recombinant glycoprotein comprising: delivering an expression vector into a cell according to an embodiment of the present disclosure, wherein the expression vector comprises a payload nucleic acid configured to encode the recombinant glycoprotein; and expressing the payload nucleic acid in the cell, thereby obtaining a plurality of recombinant glycoproteins, where at least one recombinant glycoprotein of the plurality is conjugated with a sialylated glycan.

In one aspect, the present disclosure provides a plurality of enriched recombinant glycoproteins, wherein at least 50% of the plurality of recombinant glycoproteins is configured with a sialylated glycan.

In one aspect, the present disclosure provides a cell for expressing a GlcNAc glycoprotein, being deficient in N-acetylglucosaminyltransferase I (GnTI) activity and constitutively or controllably expressing an exogenous endoglycosidase.

In one aspect, the present disclosure provides a method for glycoengineering a recombinant glycoprotein, comprising: delivering an expression vector into a cell according to an embodiment of the present disclosure, wherein the expression vector comprises a payload nucleic acid configured to encode a recombinant glycoprotein; and expressing the payload nucleic acid in the cell, thereby obtaining a plurality of recombinant glycoproteins, where at least one recombinant glycoprotein of the plurality is conjugated with a GlcNAc glycan.

In one aspect, the present disclosure provides a plurality of enriched recombinant glycoproteins, wherein at least 50% of the plurality of recombinant glycoproteins is configured with a GlcNAc glycan.

### BRIEF DESCRIPTION OF THE DRAWINGS

FIG. 1 is a graphic representation of a flow cytometry assay showing the exemplary results of an SNA staining assay conducted to examine the exemplary Adalimumab produced by using the exemplary CHO cell (SWG-006) of the present disclosure. A comparative cell transfected with B4GALT1 and ST6GAL1 configured to be expressed in separate transcriptions, and an untransfected cell (i.e., a parental cell, as a negative control). In this particular exemplary embodiment, the Adalimumab antibodies produced by the exemplary cell (SWG-006) of the present disclosure had a 5.44% SNA-positive population. The Adalimumab antibodies produced by the comparative cell had a 4.58% SNA-positive population. The parental cell expressed 0.022% SNA-positive Adalimumab antibodies.

FIG. 2 is a graphic representation of a flow cytometry assay showing the results of an SNA staining assay conducted to examine the Adalimumab produced by using the exemplary SWG-006 CHO cell of the present disclosure, the exemplary SWG-015 cell of the present disclosure, and untransfected cells (i.e., a parental cell, as a negative control). The Adalimumab antibodies produced by the SWG-006 cells of the present disclosure had a 39.8% SNA-positive population. The Adalimumab antibodies produced by the SWG-015 cells of the present disclosure had a 28.5% SNA-positive population. The parental cell expressed 0.25% SNA-positive Adalimumab antibodies.

FIG. 3A is a graphic representation of an LS/MS-MS spectrum conducted to demonstrate the glycoforms of the Adalimumab antibodies produced by an exemplary stabilized clone SAII-A3 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3B is a graphic representation of an LS/MS-MS spectrum conducted to demonstrate the glycoforms of the exemplary Adalimumab antibodies produced by an exemplary stabilized clone SAI-G1 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3C is a graphic representation of an LS/MS-MS spectrum conducted to demonstrate the glycoforms of the exemplary Adalimumab antibodies produced by an exemplary stabilized clone SAI-D4 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3D is a graphic representation of an LS/MS-MS spectrum conducted to demonstrate the glycoforms of the exemplary Adalimumab antibodies produced by an exemplary stabilized clone SAI-F12 of the CHO SWG-006 cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3E is a graphic representation of an LS/MS-MS spectrum conducted to demonstrate the glycoforms of the exemplary Adalimumab antibodies produced by a parental CHO cell. No sialylated glycans were observed. The Diamond shape represents sialic acid; the open circle shape represents galactose; the square shape represents GlcNAc; the solid circle shape represents mannose; the triangle shape represents fucose.

FIG. 4A is a graphic representation of an LS/MS-MS spectrum conducted to investigate the glycoforms of a commercial product of Adalimumab (Humira®). No sialylated glycans were observed. The Diamond shape represents sialic acid; the open circle shape represents galactose; the square shape represents GlcNAc; the solid circle shape represents mannose; the triangle shape represents fucose.

FIG. 4B is a graphic representation of an LS/MS-MS spectrum conducted to demonstrate the glycoforms of the Adalimumab antibodies produced by a parental CHO cell. No sialylated glycans were observed. The Diamond shape represents sialic acid; the open circle shape represents galactose; the square shape represents GlcNAc; the solid circle shape represents mannose; the triangle shape represents fucose.

FIG. 4C is a graphic representation of an LS/MS-MS spectrum conducted to demonstrate the glycoforms of the Adalimumab antibodies produced by an exemplary stabilized clone SAII-A3 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 5A is a graphic representation of the data of an intact protein mass (IPM) analysis conducted to investigate the glycoforms of the chMC81370 antibodies produced by a parental HEK293T cell. No sialylated glycans were observed. The Diamond shape represents sialic acid; the open circle shape represents galactose; the square shape represents GlcNAc; the solid circle shape represents mannose; the triangle shape represents fucose.

FIG. 5B is a graphic representation of the data of an intact protein mass (IPM) analysis conducted to demonstrate the glycoforms of the chMC81370 antibodies produced by an exemplary HEK293 cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 6 is a graphic representation of the data of a glycoform analysis conducted to investigate the chMC81370 antibodies produced by a wild-type cell (i.e. a parental cell) and an exemplary HEK293 cell according to the present disclosure (an “engineered” cell). Left: percentage of fucosylated glycan; Middle: percentage of galactosylated glycan; Right: percentage of sialylated glycan.

FIG. 7 is a graphic representation of the data of an intact protein mass (IPM) analysis conducted to demonstrate the glycoforms of the chMC81370 antibodies produced by an exemplary HEK293 cell according to the present disclosure, untreated (top), treated with Streptococcus pneumonia α2-3 neuraminidase (middle), or treated with Clostridium perfringens neuraminidase (bottom). The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 8 is a graphic representation of the data of an intact protein mass (IPM) analysis conducted to demonstrate the glycoforms of the chMC81370 antibodies produced by a comparative cell transfected with a hB4GalT1 gene but not hST6GAL1 gene. The data shows that the antibodies had increased galactosylation generated from cells with knock-in of hB4GalT1 only but did not show terminal sialylation. The Diamond shape represents sialic acid; the open circle shape represents galactose; the square shape represents GlcNAc; the solid circle shape represents mannose; the triangle shape represents fucose.

FIG. 9A is a graphic representation of the data of an intact protein mass (IPM) analysis conducted to investigate the changes in glycoforms of the chMC81370 antibodies produced by an exemplary HEK293 cell according to the present disclosure over five days. Top: Day 0 to Day 3 (samples collected on Day 3); Bottom: Day 4 to Day 5 (samples collected on Day 5). The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 9B is a graphic representation of the data of an intact protein mass (IPM) analysis conducted to investigate the changes in glycoforms of the chMC81370 antibodies produced by a comparative FUT8 knock-out cell over five days. Top: Day 0 to Day 3 (samples collected on Day 3); Bottom: Day 4 to Day 5 (samples collected on Day 5). The Diamond shape represents sialic acid; the open circle shape represents galactose; the square shape represents GlcNAc; the solid circle shape represents mannose; the triangle shape represents fucose.

FIG. 10 is a graphic representation of the data of an intact protein mass (IPM) analysis conducted to investigate the changes in glycoforms of the chMC81370 antibodies produced by an exemplary HEK293 cell according to the present disclosure over three days. Samples were collected on Day 1 (Top), Day 2 (Middle), and Day 3 (Bottom) respectively. The Diamond shape represents sialic acid; the open circle shape represents galactose; the square shape represents GlcNAc; the solid circle shape represents mannose; the triangle shape represents fucose.

FIG. 11 is a graphic representation of ELISA data showing the binding affinity of the antibodies produced by a wild-type cell (i.e. a parental cell) and an exemplary HEK293 cell according to the present disclosure (an “SCT-enriched” cell) to FcγIIA receptors (Left), FcγIIB receptors (Middle), and FcγIIIA receptors (Right). The binding of antibody was measured by anti-human IgG Fc antibody conjugated with HRP.

FIG. 12A presents a result of protein electrophoresis showing that the chMC18370 antibodies expressed by an exemplary HEK293 cell according to the present disclosure (Endo H KI “+”) and a parental GnTI-KO EXPI293 cell (Endo H LI “−”) are different in molecular weight as band shifting was observed.

FIG. 12B presents a result of a transglycosylation assay showing that the chMC18370 antibodies expressed by an exemplary Endo H knock-in cell, according to the present disclosure, had a high efficiency of transglycosylation. Transglycosylation was observed within 15 minutes.

FIG. 12C is a graphic representation of the data of an intact protein mass (IPM) analysis conducted to investigate the glycoforms of the chMC81370 antibodies produced by a parental cell (GnTI-KO EXPI293 cell). The square shape represents GlcNAc, and the solid circle shape represents mannose.

FIG. 12D is a graphic representation of the data of an intact protein mass (IPM) analysis conducted to investigate the glycoforms of the chMC81370 antibodies produced by an exemplary Endo H knock-in cell according to the present disclosure. The square shape represents GlcNAc, and the solid circle shape represents mannose.

FIG. 12E is a graphic representation of the data of a glycoform analysis conducted to investigate the chMC81370 antibodies produced by a GnTI-KO EXPI293 cell, an exemplary Endo H knock-in according to the present disclosure, and an exemplary Endo S2 knock-in cell according to the present disclosure. Left: percentage of mannose type glycan; Right: percentage of mono-GlcNAc glycan.

FIG. 13A presents a result of protein electrophoresis showing that the chMC18370 antibodies expressed by an exemplary HEK293 cell according to the present disclosure (Endo S2 KI “+”) and a parental GnTI-KO EXPI293 cell (Endo S2 KI “−”) are different in molecular weight as band shifting was observed.

FIG. 13B presents a result of a transglycosylation assay showing that the chMC18370 antibodies expressed by an exemplary Endo S2 knock-in cell, according to the present disclosure, had a high efficiency of transglycosylation. Transglycosylation was observed within 15 minutes.

FIG. 13C is a graphic representation of the data of an intact protein mass (IPM) analysis conducted to investigate the glycoforms of the chMC81370 antibodies produced by an exemplary Endo S2 knock-in cell according to the present disclosure. The square shape represents GlcNAc.

FIG. 14A is a graphic representation of ELISA data showing the binding affinity of the antibodies produced by an exemplary HEK293 cell according to the present disclosure (“SCT-enriched”), and an exemplary Endo H knock-in cell with a transglycosylation according to the present disclosure (“SCT homogeneous”).

FIG. 14B is a graphic representation of the data of an ADCC reporter assay showing the comparison between the antibodies produced by a wild-type cell (i.e. a parental cell), an exemplary HEK293 cell according to the present disclosure (“SCT-enriched”), and an exemplary Endo H knock-in cell with a transglycosylation according to the present disclosure (“SCT homogeneous”).

### DETAILED DESCRIPTION OF THE INVENTION

Glycosylation is a common post- or co-translational modification found on most cell proteins, especially surface proteins. The importance of a glycan profile (i.e., glycoform) of a glycoprotein and the impact on therapeutic effects has been recognized. Taking therapeutic antibodies as an example, binding between an antibody and a target cell or a pathogen triggers a variety of downstream immune functions, including phagocytosis, cellular cytotoxicity, vaccinal effect, complement activation, etc. These immune cell-based responses require the binding of the antibody Fc domain to specific Fc receptors on immune cells, in which the glycoform of the antibody is believed to be critical. For instance, it is believed that the IgG N-glycosylation at N297 on the constant region of the heavy chain is critical in the binding between the IgG and the FcγIIIA receptor on NK cells that results in the activation of antibody-dependent cellular cytotoxicity (ADCC). Therefore, it is important to engineer and obtain an optimized glycoform to improve the efficacy and safety of therapeutic glycoproteins.

Conventional mammalian cell lines used for glycoprotein production usually produce a mixture of glycoforms with core-fucosylated bi-antennary complex-type glycans, which are not optimal for binding an Fcγ receptor because of the inhibitory function of core-fucosylation or the off-target delivery caused by terminal galactosylation. In comparison, an α2-6 linked sialyl complex type (SCT) glycan provides enhanced binding to FcγIIIA receptors and FcγIIA receptors, which are associated with ADCC, antibody-dependent cellular phagocytosis (ADCP), and vaccinal effect. In addition, mono-GlcNAc (N-Acetylglucosamine) and GlcNac-Fuc-bearing glycoforms are good acceptor substrates for endoglycosidase (glycosynthase)-mediated transglycosylation and are, therefore, beneficial for engineering a desired glycan chain. Accordingly, efforts must be employed to modify the glycoforms.

### Cell

### Cells for Expressing a Sialylated Glycoprotein

One aspect of the present disclosure provides a cell for expressing a sialylated glycoprotein, which expresses constitutively and/or controllably an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity. As used herein, “translated in close proximity” describes the translation event of the exogenous sialyltransferase catalytic peptide and the translation event of the transcription of the exogenous galactosyltransferase catalytic peptide happen closely to each other both temporally and spatially.

In some embodiments, the exogenous sialyltransferase catalytic peptide and the exogenous galactosyltransferase catalytic peptide are expressed in a single transcript. As used herein, “in a single transcript” means the transcription of the exogenous sialyltransferase catalytic peptide and the transcription of the exogenous galactosyltransferase catalytic peptide are performed transcriptionally under the same promoter or the DNA encoding the exogenous sialyltransferase catalytic peptide and the DNA encoding the exogenous galactosyltransferase catalytic peptide are transcribed into a single mRNA molecule.

The present disclosure contemplates that translating the exogenous sialyltransferase catalytic peptide and the exogenous galactosyltransferase catalytic peptide in proximity to each other or expressing the two peptides in a single transcription provides higher sialylation to the glycoprotein. Without wishing to be bound by any theories, since the catalytic reaction exerted by a galactosyltransferase generates a galactosylated glycan, which is the substrate of a sialyltransferase, translating the two enzymes in proximity or expressing them in a single transcription ensures the two catalytic reactions happen closely or nearly simultaneously, thereby higher sialylation efficiency.

Ribosomal shifting approach. In some embodiments, the cell of the present disclosure comprises a first nucleic acid configured to express the exogenous sialyltransferase catalytic peptide; and a second nucleic acid configured to express the exogenous galactosyltransferase catalytic peptide, wherein the first nucleic and the second nucleic acid are controlled under the same promoter. In some embodiments, the first nucleic acid and the second nucleic acid are connected to each other via a connecting nucleic acid, which is configured to encode a ribosomal shifting peptide. A ribosomal shifting peptide is configured to render ribosomal skipping, where the ribosomal shifting peptide prevents the ribosome from covalently linking a newly inserted amino acid while continuing the translation, resulting in co-translational cleavage of a polyprotein into separate peptides/proteins. In some embodiments, the ribosomal shifting peptide comprises an amino acid sequence of DxExNPGP (SEQ ID NO: 28), wherein x denotes any amino acid, D denotes aspartic acid, E donates glutamic acid, N denotes asparagine, P denotes proline, and G denotes glycine. In certain embodiments, the ribosomal shifting peptide comprises an amino acid sequence as set forth in SEQ ID NO: 06, SEQ ID NO: 07, SEQ ID NO: 08, or SEQ ID NO: 09. In certain embodiments, the connecting nucleic acid comprises a sequence as set forth in SEQ ID NO: 19, SEQ ID NO: 20, SEQ ID NO: 21, or SEQ ID NO: 22.

Fusion protein approach. Alternatively, the exogenous sialyltransferase catalytic peptide and the exogenous galactosyltransferase catalytic peptide are, in some embodiments, configured to be expressed into a fusion protein. In some embodiments, the fusion protein comprises a first portion having the sialyltransferase catalytic peptide and a second portion having the galactosyltransferase catalytic peptide, wherein the first portion and the second portion are connected to each other via a cleavable linker, and the cleavable linker is configured to be cleavable post-translation of the fusion protein, thereby upon cleavage releasing the sialyltransferase catalytic peptide and the galactosyltransferase catalytic peptide as separate proteins.

In some embodiments, the sialyltransferase catalytic peptide is an alpha-2,6-sialyltransferase. In some embodiments, the sialyltransferase is a beta-galactoside alpha-2,6-sialyltransferase 1. In certain embodiments, the sialyltransferase catalytic peptide comprises a product of an ST6Gal1 gene or a PspST gene. In certain embodiments, the sialyltransferase catalytic peptide comprises an amino acid sequence as the sequence set forth in SEQ ID NO: 01 or SEQ ID NO: 02. Yet in certain embodiments, the sialyltransferase catalytic peptide comprises an amino acid sequence as set forth in SEQ ID NO: 03 or SEQ ID NO: 04.

In some embodiments, the galactosyltransferase catalytic peptide is a beta-1,4-galactosyltransferase 1. In certain embodiments, the galactosyltransferase catalytic peptide comprises a product of a B4GALT1 gene. In certain embodiments, the galactosyltransferase catalytic peptide comprises an amino acid sequence as set forth in SEQ ID NO: 05.

In some embodiments, the cell comprises a first nucleic acid configured to express the exogenous sialyltransferase catalytic peptide, wherein the first nucleic acid is derived from an ST6Gal1 gene or a PspST gene. As used herein, “derived from” describes that the first nucleic acid comprises a nucleotide sequence exactly the same as a reference gene; for example, the first nucleic acid might comprise a nucleotide sequence as set forth in SEQ ID NO: 10 or SEQ ID NO: 11. In some other situations, the “derived from” describes that the first nucleic acid comprises a nucleotide sequence modified from the reference gene, provided that the product of the modified nucleotide sequence exerts the same or similar catalytic functionality as the product of the reference gene. The modification might be made for accommodating codon usage or optimizing transcription/translation efficiency or accuracy in a host cell. In other examples, the modification adds a signal peptide directing the gene product to a certain place within or out of the cells. For example, the first nucleic acid can be derived from a PspST gene that the first nucleic acid comprises a nucleotide sequence encoding a product of the PspST gene and a nucleotide sequence encoding a signal peptide that directs the gene product to the Golgi body where glycosylation takes place. The signal peptide, in certain embodiments, can be the signal peptide of an ST6Gal1 gene or a B4GALT1 gene. In such embodiments, the first nucleic acid comprises a nucleotide sequence as set forth in SEQ ID NO: 12 or SEQ ID NO: 13.

In some embodiments, the cell comprises a second nucleic acid configured to express the exogenous galactosyltransferase catalytic peptide, wherein the second nucleic acid is derived from a B4GALT1 gene. In certain embodiments, the second nucleic acid comprises a nucleotide sequence as set forth in SEQ ID NO: 14.

In some embodiments, the first nucleic acid and the second nucleic acid are transcriptionally controlled under the same promoter, which can be a constitutive promoter or an activable promoter. The constitutive promoter can be but is not limited to a CMV promoter, a T7 promoter, a Human Elongation Factor 1 Alpha (EF1α) promoter, a Chicken β-Actin (CAG) promoter, or an SV40 promoter. The activable promoter provides a controllable expression of the exogenous sialyltransferase and the exogenous galactosyltransferase. The activable promoter can be but is not limited to a Tetracycline-Inducible Promoter (activable by doxycycline) or a dihydrofolate reductase (DHFR) gene promoter (used to select and amplify gene expression).

Parental cell. In some embodiments, the cell of the present disclosure is derived from a parental cell (e.g., a wild-type cell), which can be a mammalian cell. In some embodiments, the parental cell does not have an endogenous sialyltransferase and/or an endogenous galactosyltransferase. In some other embodiments, the parental cell might have an endogenous sialyltransferase and/or an endogenous galactosyltransferase. Nevertheless, the present disclosure surprisedly discovers that overexpression of the endogenous sialyltransferase and/or the endogenous galactosyltransferase does not increase the sialylation as expressing exogenous ones can achieve. In some embodiments, the parental cell and/or the cell of the present disclosure are deficient in fucosyltransferase 8 activity. In certain embodiments, the parental cell and/or the cell of the present disclosure are deficient in a FUT8 gene encoding a product of the fucosyltransferase activity. In some embodiments, the parental cell and/or the cell of the present disclosure are or are derived from a Chinese hamster ovary (CHO) cell or a HEK293 cell. The CHO cell can be but is not limited to EXPICHO®, CHO-KI™, CHO-C®, CHOZN™, and CHOK1Q™. In some embodiments, “cell” used herein is interchangeably with “cell line.”

Cells for expressing a recombinant glycoprotein. In some embodiments, the cell further comprises a payload nucleic acid configured to encode a recombinant glycoprotein, and the expression of the payload nucleic acid is transcriptionally controlled by a constitutive or an activable promoter. The constitutive promoter and the activable promoter are as described above and herein. In some embodiments, the recombinant glycoprotein expressed or produced by the cell is conjugated with a glycan. In certain embodiments, the glycan can be an N-linked glycan (i.e., N-glycan) or an O-linked glycan (i.e., O-glycan).

Glycan and glycoprotein. The cell of the present disclosure is capable of expressing a sialylated glycoprotein. In some embodiments, the sialylated glycoprotein comprises a sialyl complex type (SCT) glycan, which can be conjugated to the protein via a lysine residue (i.e., O-glycan) or an asparagine residue (i.e., N-glycan). In some embodiments, the cell of the present disclosure is configured to express an SCT-enriched protein. As used herein, “SCT-enriched” describes that the proteins expressed by the cell of the present disclosure have a higher SCT glycan percentage compared to the same proteins expressed by a parental cell used to produce the cells of the present disclosure. In some embodiments, the SCT glycan percentage is 10%, 20%, 30%, 40%, 50%, 60%, 70%, 80%, 90%, 100%, 200%, 300%, 400%, 500% higher than that of the proteins expressed by a parental cell, or any range defined by the foregoing endpoints, such as 10% to 500%, 10% to 400%, 10% to 300%, 10% to 200%, 10% to 100%, 10% to 90%, 10% to 80%, 10% to 70%, 10% to 60%, 10% to 50%, 10% to 40%, 10% to 30%, 10% to 20%, 30% to 500%, 30% to 400%, 30% to 300%, 30% to 200%, 30% to 100%, 30% to 90%, 30% to 80%, 30% to 70%, 30% to 60%, 30% to 50%, or 30% to 40%. In certain embodiments, the SCT glycan percentage is enriched to 1.2, 1.5, 2, 2.5, 3, 3.5, 4, 4.5, 5, 5.5, 6, 6.5, 7, 7.5, 8, 8.5, 9, 9.5, or 10 times the SCT glycan percentage of the proteins expressed by a parental cell, or any range defined by the foregoing endpoints, such as 1.2 to 10 times, 1.2 to 9 times, 1.2 to 8 times, 1.2 to 7 times, 1.2 to 6 times, 1.2 to 5 times, 1.2 to 4 times, 1.2 to 3 times, 1.2 to 2 times, 2 to 10 times, 2 to 9 times, 2 to 8 times, 2 to 7 times, 2 to 6 times, 2 to 5 times, 2 to 4 times, 2 to 3 times, 5 to 10 times, 5 to 9 times, 5 to 8 times, 5 to 7 times, or 5 to 6 times. In certain embodiments, the glycan is a mono-antennary or bi-antennary α2-6 sialyl complex type (SCT) glycan. In some embodiments, the glycan is a galactose-rich SCT glycan with or without core-fucose. In some other embodiments, the glycan is a fully galactosylated SCT glycan with or without core-fucose.

In certain embodiments, the glycoprotein (e.g., a recombinant glycoprotein) is an antibody or an antigen-binding fragment thereof. A basic antibody unit is a heterotetrameric glycoprotein composed of two identical light (L) chains and two identical heavy (H) chains. Each L chain is linked to an H chain by at least one (and typically one) covalent disulfide bond, while the two H chains are linked to each other by one or more disulfide bonds depending on the H chain isotype. Each H and L chain also has regularly spaced intrachain disulfide bridges. Each H chain has at the N-terminus a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable domains. The pairing of a V_{H} and V_{L} together forms a single antigen-binding site.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ), and mu (μ), respectively. The γ and α classes are further divided into subclasses on the basis of relatively minor differences in CH sequence and function, e.g., humans express the following subclasses: IgG1, IgG2, IgG3, IgG4, IgA1, and IgA2. It will be appreciated that mammals encoding multiple Ig isotypes will be able to undergo isotype class switching.

An “antibody fragment” or “antigen-binding fragment of an antibody” is a polypeptide comprising or consisting of a portion of an intact antibody, preferably the antigen binding or variable region of the intact antibody. Examples of antibody fragments include an F(ab′)₂ fragment, an Fv fragment, a single-chain Fv (ScFv) antibody, a diabody, minibody, nanobody (V_{H}H), and a linear antibody (see U.S. Pat. No. 5,641,870; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fe” fragment, a designation reflecting the ability to crystallize readily. The Fab fragment consists of an entire L chain along with the variable region domain of the H chain (V_{H}) and the first constant domain of one heavy chain (C_{H1}). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Both the Fab and F(ab′)₂ are examples of “antigen-binding fragments.” Fab′ fragments differ from Fab fragments by having an additional few residue at the carboxy terminus of the CH1 domain, including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions (i.e., the C_{H2} and C_{H3} domains of IgG) of both H chains held together by disulfides. The effector functions of antibodies are determined by sequences in the Fc region. The Fc domain is the portion of the antibody recognized by cell receptors, such as the FcR, and to which the complement-activating protein, C1q, binds. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure).

“Fv” is the minimum antibody fragment that contains a complete antigen-recognition and antigen-binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. Preferably, the sFv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993). Other antibody fragments and molecules comprising the same include, for example, linear antibodies, tandem scFv, scFv-Fc, tandem scFv-Fc, scFv dimer, scFv-zipper, diabody-Fc, diabody-C_{H3}, scDiabodies, scDiabody-Fc, scDiabody-CH3, nanobodies, TandAbs, minibodies, miniantibodies, triabodies, tetrabodies, scFab, Fab-scFv, Fab-scFv-Fc, scFv-CH-CL-scFv, and F(ab′)₂-scFv2, all of which are also contemplated herein.

In the embodiments that the glycoprotein is an antibody or an antigen-binding fragment thereof, the glycan can be located on a heavy chain or an Fc region. It is important to note that the glycosylation site of an antibody that regulates the antibody functions can differ from subtype to subtype. For example, the preBCR assembly is important for B cell development and is critically regulated by the N-glycan at N46 on μHC. The N-glycan at N402 on μHC has been linked to antibody oligomerization and complement activation. Besides, IgG N-glycosylation at N297 on γHC plays a critical role in complement activation and Fcγ receptor activation leading to various effector functions. Therefore, in some embodiments, the glycan can be located on N46, N402, and/or N297 of a heavy chain.

In certain embodiments, the glycoprotein is Adalimumab (Humira®), Adalimumab-atto (Amjevita®), Rituximab (Rituxan®), Rituximab-atto (Truxima®), Cetuximab (Erbitux®), Bevacizumab (Avastin®), Infliximab (Remicade®), Trastuzumab (Herceptin®), Pembrolizumab (Keytruda®), Etanercept (Enbrel®), Ipilimumab (Yervoy®), Ofatumumab (Arzerra), Golimumab (Simponi®), Atezolizumab (Tecentriq®), Ocrelizumab (OCREVUS®), Durvalumab (Durvalumab®), Avelumab (Bavencio®), Nivolumab (Opdivo®), Pertuzumab (Perjeta®), Obinutuzumab (Gazyva®), Gazyvaro Infliximab (Remicade®), or Trastuzumab emtansine (Kadcyla®).

In certain embodiments, the glycoprotein is an immunogenic protein, such as a viral envelop protein, or a spike protein of a virus. For example, the immunogenic protein can be but is not limited to an influenza hemagglutinin or a SARS-COV-2 spike protein.

### Cells for Expressing a GlcNAc Glycoprotein

One aspect of the present disclosure provides a cell for expressing a GlcNAc glycoprotein, wherein the cell is deficient in N-acetylglucosaminyltransferase I (GnTI) activity and constitutively or controllably expresses an exogenous endoglycosidase. As described herein, “GlcNAc glycoprotein” describes that the glycan conjugated on the glycoprotein is composed of mainly GlcNAc. In some embodiments, the GlcNAc glycan can be a mono-GlcNAc glycan or a GlcNAc-Fuc glycan. In some embodiments, the glycoprotein is as described above and herein.

The exogenous endoglycosidase can be but is not limited to an endoglycosidase H (Endo H) or an endoglycosidase S2 (Endo S2). In certain embodiments, the exogenous endoglycosidase comprises an amino acid sequence as set forth in SEQ ID NO: 15 or SEQ ID NO: 16. In some certain embodiments, the cell comprises a nucleic acid configured to encode the exogenous endoglycosidase, wherein the nucleic acid might comprise a nucleotide sequence as set forth in SEQ ID NO: 17 or SEQ ID NO: 18. The nucleic acid can be controlled under a constitutive or an activable promoter. The constitutive promoter can be but is not limited to a CMV promoter, a T7 promoter, an EF1A promoter, a CAG promoter, or an SV40 promoter. The activable promoter provides a controllable expression of the exogenous sialyltransferase and the exogenous galactosyltransferase. The activable promoter can be but is not limited to a Tetracycline-Inducible Promoter (activable by doxycycline) or a dihydrofolate reductase (DHFR) gene promoter (used to select and amplify gene expression).

As described herein, “deficient in N-acetylglucosaminyltransferase I (GnTI) activity” means that the cell does not have a functioning N-acetylglucosaminyltransferase I, which can result from knocking out an endogenous gene encoding the N-acetylglucosaminyltransferase I or a mutation at the gene causing missense, nonsense, or frameshift silent of the gene. Without wishing to be bound by any theories, a cell deficient in GnTI activity produces a mannose-rich glycan, which is preferentially cleaved by an endoglycosidase, thereby generating a GlcNAc glycoprotein.

Parent cell. In some embodiments, the cell of the present disclosure is derived from a parent cell, which can be a mammalian cell. In some embodiments, the parent cell is deficient in N-acetylglucosaminyltransferase I (GnTI) activity. In some other embodiments, the parent cell is not deficient in acetylglucosaminyltransferase I (GnTI) activity, while the cell of the present disclosure is genetically engineered to be deficient in GnTI activity. In some embodiments, the parent cell and/or the cell of the present disclosure are deficient in fucosyltransferase 8 activity. In certain embodiments, the parent cell and/or the cell of the present disclosure are deficient in a FUT8 gene encoding a product of the fucosyltransferase activity. In some embodiments, the parent cell and/or the cell of the present disclosure are or are derived from a Chinese hamster ovary (CHO) cell or a HEK293 cell. The CHO cell can be but is not limited to EXPICHO®, CHO-K1™, CHO-C®, CHOZN™, and CHOK1Q™, and the HEK293 cell can be Expi293F™ GnTI KO.

Cells for expressing a recombinant glycoprotein. In some embodiments, the cell further comprises a payload nucleic acid configured to encode a recombinant glycoprotein, and the expression of the payload nucleic acid is controlled by a constitutive or an activable promoter. The constitutive promoter and the activable promoter are as described above and herein. In some embodiments, the recombinant glycoprotein expressed or produced by the cell is conjugated with a glycan. In certain embodiments, the glycan can be an N-linked glycan.

### Production of the Cells of the Present Disclosure

The production of the cells according to an embodiment of the present disclosure entails engineering a parent cell so that the engineered cell expresses the exogenous enzymes. In some embodiments, the production of the cells entails engineering a parent cell to knock in and/or knock out a gene of interest. In some embodiments, the engineering can be performed using transfection approaches or gene editing approaches, which can be either stable or transient.

### Transfection

The transfection can be conducted using any conventional methodologies with the consideration of high transfection efficiency, minimal cell toxicity, low or no significant effects on normal physiology, and ease of operation.

In some embodiments, the transfection can be performed using virus-mediated methods. The virus-mediated methods use a viral vector to bring a nucleic acid configured to encode a desired gene product into a host cell. Some commonly used viral vectors include but are not limited to Adenovirus, Adeno-associated virus, retrovirus murine leukemia virus, Herpes simplex virus, Vaccinia virus, and Sindbis virus. Virus-mediated methods are highly effective, given the infectious nature of the viral particles. The downsides of these methods, however, lie in the concerns of immunogenicity and cytotoxicity. Nevertheless, many virus-mediated methods have been well-studied and developed to ease the concerns. Besides, in some embodiments, the cell of the present disclosure is used to produce a desired glycoprotein in which uses, the transfection is not performed on a patient or a human subject directly. Therefore, the immunogenicity and cytotoxicity concerns are less critical for the present disclosure.

In some embodiments, the transfection can be performed using chemical methods, such as using a cationic polymer, calcium phosphate, a cationic lipid, or a cationic amino acid. Specific examples of chemical methods include but are not limited to using DEAE-dextran, polyethyleneimine, dendrimer, polybrene, calcium phosphate, LIPOFECTIN™, DOTAP, LIPOFECTAMINE®, CTAB/DOPE, or DOTMA. The underlying principle of those methods is using those positively charged chemicals to make nucleic acid/chemical complexes with negatively charged nucleic acids. The nucleic acid/chemical complexes are believed to be attracted to the negatively charged cell membrane and pass through it eventually via endocytosis, phagocytosis, or both. The efficiency of those chemical methods can be affected by several factors including the nucleic acid/chemical ratio, pH value of the environment, and cell membrane conditions.

In some embodiments, the transfection can be performed using physical methods, such as direct microinjection, biolistic particle delivery, electroporation, laser-based transfection, ultrasound transfection, and magnetofection. Those methods can be useful but usually require a higher skill level and are labor-intensive.

### Genome Editing

In some embodiments, the engineering can be performed using genome editing. The genome editing methods can be non-targeted gene editing, such as Sleeping Beauty transposon or I-SceI meganuclease-mediated transposon, or targeted gene editing, such as using transcription activator-like effector nuclease (TALEN) or clustered regularly interspaced short palindromic repeats (CRISPR)/CRISPR-associated protein. Gene editing provides integration of the exogenous nucleic acid into the genome of the host cell (parent cell).

The targeted gene editing methods induce DNA double-strand breaks (DSBs) at a targeted locus, which enable the opportunities to alter the function (inserting an exogenous gene or silencing an endogenous gene) of a gene of interest. The TALEN approach entails using a chimeric molecule consisting of a transcription activator-like effector (TALE) domain and a FokI nuclease catalytic domain. The TALE domain comprises a DNA-binding motif, which can be designed to target a specific locus of the host cell's genome. Normally, a pair of two chimeric molecules, each targeting the forward and reverse directions respectively, are required. Once the TALE domains bind the targeted locus in the forward and reverse directions, the FokI nuclease catalytic domains of the pair of two chimeric molecules form a dimer, thereby activating the catalytic activity of the nuclease to cleave the DNA and generate a DSB. The CRISPR method comprises using a complex of Cas protein and a guide RNA. The guide RNA comprises a scaffold sequence for Cas-binding and a spacer (around 20 nucleotides) having a complementary sequence to a specific locus of the host cell's genome. Normally, the spacer must have a sequence that is unique and specific to the target locus to prevent off-target binding, and the target locus is followed by a protospacer adjacent motif (PAM) site, which is necessary for Cas protein recognition. Once the guide RNA/Cas protein complex binds to the target site, the Cas nuclease activity will be activated and cleave the DNA to generate a DCB.

The DSBs induced can be repaired by three different repair mechanisms, including homology-directed repair (HDR), non-homologous end joining (NHEJ), and micro-homology-mediated end joining (MMEJ). In the process of the repair mechanism, deletion or frameshift can result in gene silence, or, by using a donor vector comprising microhomology sequences homologous to a targeted locus, the repair mechanism can efficiently induce a targeted integration of exogenous genes into the genome.

### Methods for Glycoengineering a Recombinant Glycoprotein and Recombinant Glycoproteins Obtained Thereby

One aspect of the present disclosure provides a method for glycoengineering a recombinant glycoprotein. The method comprises delivering an expression vector into a cell according to an embodiment of the present disclosure, wherein the expression vector comprises a payload nucleic acid configured to encode a recombinant glycoprotein; and expressing the payload nucleic acid in the cell, thereby obtaining a plurality of recombinant glycoproteins, where at least one recombinant glycoprotein of the plurality is conjugated with a desired glycan

The glycoprotein can be as those described herein. In certain embodiments, the glycoprotein is a therapeutic antibody or an antigen-binding fragment thereof. In the embodiments that the glycoprotein is an antibody or an antigen-binding fragment thereof, the glycan can be located on a heavy chain or an Fc region. In some embodiments, the glycan is located at a glycosylation site of the antibody. In certain embodiments, the glycan can be located on N46, N402, and/or N297 of a heavy chain.

As used herein, “glycoengineering” refers to producing the recombinant glycoprotein using the cell whereby the recombinant glycoprotein carries or is conjugated with a desired glycoform or desired glycan. In some embodiments, at least 50%, 55%, 60%, 65%, 70%, 75%, 80%, 85%, 90%, 95%, 99%, or 100% of them carry the desired glycoform (e.g., a sialylated glycan or a GlcNAc glycan), or an integer ranging between any of the above two numbers, or any range defined by the foregoing endpoints, such as 50 to 100%, 50 to 99%, 50 to 95%, 50 to 90%, 50 to 85%, 50 to 80%, 50 to 75%, 50 to 70%, 50 to 65%, 50 to 60%, 50 to 55%, 60 to 100%, 60 to 99%, 60 to 95%, 60 to 90%, 60 to 85%, 60 to 80%, 60 to 75%, 60 to 70%, 60 to 65%, 70 to 100%, 70 to 99%, 70 to 95%, 70 to 90%, 70 to 85%, 70 to 80%, 70 to 75%, 80 to 100%, 80 to 99%, 80 to 95%, 80 to 90%, 80 to 85%, 90 to 100%, 90 to 99%, or 90 to 95%.

In some embodiments, the plurality of recombinant glycoproteins is conjugated with the desired glycoform (e.g., a sialylated glycan or a GlcNAc glycan) at homogeneity of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range defined by the foregoing endpoints, such as 1% to 100%, 1% to 90%, 1% to 80%, 1% to 70%, 1% to 60%, 1% to 50%, 1% to 40%, 1% to 30%, 1% to 20%, 1% to 10%, 5% to 100%, 5% to 95%, 5% to 85%, 5% to 75%, 5% to 65%, 5% to 55%, 5% to 45%, 5% to 35%, 5% to 25%, 5% to 10%, 10% to 100%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 100%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 100%, 40% to 99%, 40% to 95%, 40% to 90%, 40% to 85%, 40% to 80%, 40% to 75%, 40% to 70%, 40% to 65%, 40% to 60%, 40% to 55%, 40% to 50%, 60% to 100%, 60% to 99%, 60% to 95%, 60% to 90%, 60% to 85%, 60% to 80%, 60% to 75%, 50% to 100%, 50% to 99%, 50% to 95%, 50% to 90%, 50% to 85%, 50% to 80%, 50% to 75%, 50% to 70%, 50% to 65%, 50% to 60%, 55% to 100%, 55% to 99%, 55% to 98%, 55% to 97%, 55% to 96%, 55% to 95%, 55% to 94%, 55% to 93%, 55% to 92%, 55% to 91%, 55% to 90%, 55% to 85%, 55% to 80%, 55% to 75%, 55% to 70%, 55% to 65%, 55% to 60%, 70% to 100%, 70% to 99%, 70% to 95%, 70% to 90%, 70% to 85%, 70% to 80%, 70% to 75%, 80% to 100%, 80% to 99%, 80% to 95%, 80% to 90%, 80% to 85%, 90% to 100%, 90% to 99%, or 90% to 95%, including or excluding any foregoing numbers. All numbers can be modified by “about,” which is defined herein.

In some embodiments, the cell is according to the cell of the present disclosure described above for expressing a sialylated glycoprotein. In some embodiments, the sialylated glycoprotein comprises a sialyl complex type (SCT) glycan. In some embodiments, the method of the present disclosure is configured to express an SCT-enriched protein, which is defined as described herein. In some other embodiments, the cell is according to the cell of the present disclosure above for expressing a GlcNAc glycoprotein, which is defined as described herein.

In some embodiments, the method further comprises harvesting the recombinant glycoprotein within 200, 180, 150, 120, 100, 80, 50, 30, 20, 10, 5, or 1 hour from the expression of the payload nucleic acid, or an integer ranging between any of the above two numbers, or any range defined by the foregoing endpoints, such as 1 to 200 hours, 1 to 180 hours, 1 to 150 hours, 1 to 120 hours, 1 to 100 hours, 1 to 80 hours, 1 to 50 hours, 1 to 30 hours, 1 to 20 hours, 1 to 10 hours, 1 to 5 hours, 5 to 200 hours, 5 to 180 hours, 5 to 150 hours, 5 to 120 hours, 5 to 100 hours, 5 to 80 hours, 5 to 50 hours, 5 to 30 hours, 5 to 20 hours, or 5 to 10 hours. In some embodiments, the recombinant glycoprotein is harvested within 10, 9, 8, 7, 6, 5, 4, 3, 2, or 1 day from the expression of the payload nucleic acid, or an integer ranging between any of the above two numbers, or any range defined by the foregoing endpoints, such as 1 to 10 days, 1 to 9 days, 1 to 8 days, 1 to 7 days, 1 to 6 days, 1 to 5 days, 1 to 4 days, 1 to 3 days, or 1 to 2 days. As described herein, “from the expression of the payload nucleic acid” means starting from the time point that the expression vector is delivered into the cell, or starting from the time point that the expression vector is delivered into the cell and the promoter controlling the expression of the payload nucleic acid is activated.

In some embodiments, the expression of the payload nucleic acid is constitutive or controllable. In certain embodiments, the expression of the payload nucleic acid is controlled by a constitutive promoter or an activable promoter. The constitutive promoter can be but is not limited to a CMV promoter, a T7 promoter, an EF1A promoter, a CAG promoter, or an SV40 promoter. The activable promoter provides a controllable expression of the exogenous sialyltransferase and the exogenous galactosyltransferase. The activable promoter can be but is not limited to a Tetracycline-Inducible Promoter (activable by doxycycline) or a dihydrofolate reductase (DHFR) gene promoter (used to select and amplify gene expression).

In some embodiments, the expression vector can be delivered via gene engineering, which can be performed using transfection approaches or gene editing approaches, which can be either stable or transient. The transfection approaches and the gene editing approaches can be as those described herein for producing the cells of the present disclosure.

Another aspect of the present disclosure provides a plurality of recombinant glycoproteins, wherein at least 50% of the plurality of the recombinant glycoproteins is configured with a desired glycan, which can be a sialylated glycan or a GlcNAc glycan. The recombinant glycoprotein can be described herein. In certain embodiments, the plurality of recombinant glycoproteins is obtained by using the methods for glycoengineering a recombinant glycoprotein of the present disclosure.

In some embodiments, the plurality of recombinant glycoproteins is a plurality of enriched recombinant glycoprotein. The term “enriched” describes that the plurality of recombinant glycoproteins of the present disclosure have a higher or enriched desired glycan percentage compared to the same proteins expressed by a parental cell or a wild-type cell used to produce the cells of the present disclosure, or the plurality of recombinant glycoproteins of the present disclosure have a higher or enriched desired glycan percentage compared to the desired glycan percentage of a plurality of naturally-occurred proteins having the same or similar amino acid sequences. In some embodiments, the desired glycan percentage is enriched by at least 30%, 35%, 40%, 45%, 50%, 55%, 60%, 65%, 70%, 75%, 80%, 85%, 90%, 95%, 100%, 150%, 200%, 250%, 300%, 350%, 400%, 450%, or 500%, or an integer ranging between any of the above two numbers, or any range defined by the foregoing endpoints, such as 10% to 500%, 10% to 400%, 10% to 300%, 10% to 200%, 10% to 100%, 10% to 90%, 10% to 80%, 10% to 70%, 10% to 60%, 10% to 50%, 10% to 40%, 10% to 30%, 10% to 20%, 30% to 500%, 30% to 400%, 30% to 300%, 30% to 200%, 30% to 100%, 30% to 90%, 30% to 80%, 30% to 70%, 30% to 60%, 30% to 50%, or 30% to 40%, including or excluding any foregoing numbers. All numbers can be modified by “about,” which is defined herein. In certain embodiments, the desired glycan percentage is enriched to 1.2, 1.5, 2, 2.5, 3, 3.5, 4, 4.5, 5, 5.5, 6, 6.5, 7, 7.5, 8, 8.5, 9, 9.5, or 10 times the SCT glycan percentage of the proteins expressed by a parental cell, or an integer ranging between any of the above two numbers, or any range defined by the foregoing endpoints, such as 1.2 to 10 times, 1.2 to 9 times, 1.2 to 8 times, 1.2 to 7 times, 1.2 to 6 times, 1.2 to 5 times, 1.2 to 4 times, 1.2 to 3 times, 1.2 to 2 times, 2 to 10 times, 2 to 9 times, 2 to 8 times, 2 to 7 times, 2 to 6 times, 2 to 5 times, 2 to 4 times, 2 to 3 times, 5 to 10 times, 5 to 9 times, 5 to 8 times, 5 to 7 times, or 5 to 6 times, including or excluding any foregoing numbers. All numbers can be modified by “about,” which is defined herein.

In some embodiments, the desired glycan percentage is enriched by at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%, or an integer ranging between any of the above two numbers, or any range defined by the foregoing endpoints, such as 51% to 95%, 51% to 94%, 51% to 93%, 51% to 92%, 51% to 91%, 51% to 90%, 51% to 85%, 51% to 80%, 51% to 75%, 51% to 70%, 51% to 65%, 51% to 60%, 55% to 95%, 55% to 90%, 55% to 85%, 55% to 80%, 55% to 75%, 55% to 70%, 55% to 65%, 55% to 60%, 60% to 95%, 60% to 90%, 60% to 85%, 60% to 80%, 60% to 75%, 60% to 70%, 60% to 65%, 70% to 95%, 70% to 90%, 70% to 85%, 70% to 80%, or 70% to 75%, including or excluding any foregoing numbers. All numbers can be modified by “about,” which is defined herein.

In some embodiments, at least 50%, 55%, 60%, 65%, 70%, 75%, 80%, 85%, 90%, 95%, 99%, or 100% of the plurality of the recombinant glycoprotein carry the desired glycoform (e.g., a sialylated glycan or a GlcNAc glycan), or an integer ranging between any of the above two numbers, or any range defined by the foregoing endpoints, such as 50 to 100%, 50 to 99%, 50 to 95%, 50 to 90%, 50 to 85%, 50 to 80%, 50 to 75%, 50 to 70%, 50 to 65%, 50 to 60%, 50 to 55%, 60 to 100%, 60 to 99%, 60 to 95%, 60 to 90%, 60 to 85%, 60 to 80%, 60 to 75%, 60 to 70%, 60 to 65%, 70 to 100%, 70 to 99%, 70 to 95%, 70 to 90%, 70 to 85%, 70 to 80%, 70 to 75%, 80 to 100%, 80 to 99%, 80 to 95%, 80 to 90%, 80 to 85%, 90 to 100%, 90 to 99%, or 90 to 95%. All numbers can be modified by “about,” which is defined herein.

In some embodiments, the plurality of recombinant glycoproteins is conjugated with the sialylated glycan at homogeneity of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range defined by the foregoing endpoints, such as 1% to 100%, 1% to 90%, 1% to 80%, 1% to 70%, 1% to 60%, 1% to 50%, 1% to 40%, 1% to 30%, 1% to 20%, 1% to 10%, 5% to 100%, 5% to 95%, 5% to 85%, 5% to 75%, 5% to 65%, 5% to 55%, 5% to 45%, 5% to 35%, 5% to 25%, 5% to 10%, 10% to 100%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 100%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 100%, 40% to 99%, 40% to 95%, 40% to 90%, 40% to 85%, 40% to 80%, 40% to 75%, 40% to 70%, 40% to 65%, 40% to 60%, 40% to 55%, 40% to 50%, 60% to 100%, 60% to 99%, 60% to 95%, 60% to 90%, 60% to 85%, 60% to 80%, 60% to 75%, 50% to 100%, 50% to 99%, 50% to 95%, 50% to 90%, 50% to 85%, 50% to 80%, 50% to 75%, 50% to 70%, 50% to 65%, 50% to 60%, 55% to 100%, 55% to 99%, 55% to 98%, 55% to 97%, 55% to 96%, 55% to 95%, 55% to 94%, 55% to 93%, 55% to 92%, 55% to 91%, 55% to 90%, 55% to 85%, 55% to 80%, 55% to 75%, 55% to 70%, 55% to 65%, 55% to 60%, 70% to 100%, 70% to 99%, 70% to 95%, 70% to 90%, 70% to 85%, 70% to 80%, 70% to 75%, 80% to 100%, 80% to 99%, 80% to 95%, 80% to 90%, 80% to 85%, 90% to 100%, 90% to 99%, or 90% to 95%, including or excluding any foregoing numbers. All numbers can be modified by “about,” which is defined herein.

In the embodiments that the cell is according to the cell of the present disclosure above for expressing a GlcNAc glycoprotein, the method of the present disclosure can further comprise a transglycosylation step. The transglycosylation step can comprise collecting the plurality of recombinant glycoproteins expressed in the preceding steps of the method and incubating the plurality of recombinant glycoproteins with an endoglycosidase and a donor glycan, thereby conjugating the donor glycan onto at least one glycoprotein of the plurality of recombinant glycoproteins (i.e., a transglycosylation reaction).

In some embodiments, the donor glycan comprises an oxazoline moiety configured to react with the endoglycosidase thereby conjugating the donor glycan onto at least one glycoprotein of the plurality of recombinant glycoproteins. In certain embodiments, the donor glycan is a sialyl complex type (SCT) glycan. In some embodiments, the endoglycosidase is an endoglycosidase H (Endo H), an endoglycosidase S2 (Endo S2), or a derivative thereof. In some embodiments, the derivative of the Endo S2 comprises an Endo S2 D184 mutant, including but not limited to, a D184M mutant or a D184Q mutant.

In some embodiments, the transglycosylation step generates a plurality of recombinant glycoprotein conjugated with the donor glycan at a homogeneity of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range defined by the foregoing endpoints, such as 1% to 100%, 1% to 90%, 1% to 80%, 1% to 70%, 1% to 60%, 1% to 50%, 1% to 40%, 1% to 30%, 1% to 20%, 1% to 10%, 5% to 100%, 5% to 95%, 5% to 85%, 5% to 75%, 5% to 65%, 5% to 55%, 5% to 45%, 5% to 35%, 5% to 25%, 5% to 10%, 10% to 100%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 100%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 100%, 40% to 99%, 40% to 95%, 40% to 90%, 40% to 85%, 40% to 80%, 40% to 75%, 40% to 70%, 40% to 65%, 40% to 60%, 40% to 55%, 40% to 50%, 60% to 100%, 60% to 99%, 60% to 95%, 60% to 90%, 60% to 85%, 60% to 80%, 60% to 75%, 50% to 100%, 50% to 99%, 50% to 95%, 50% to 90%, 50% to 85%, 50% to 80%, 50% to 75%, 50% to 70%, 50% to 65%, 50% to 60%, 55% to 100%, 55% to 99%, 55% to 98%, 55% to 97%, 55% to 96%, 55% to 95%, 55% to 94%, 55% to 93%, 55% to 92%, 55% to 91%, 55% to 90%, 55% to 85%, 55% to 80%, 55% to 75%, 55% to 70%, 55% to 65%, 55% to 60%, 70% to 100%, 70% to 99%, 70% to 95%, 70% to 90%, 70% to 85%, 70% to 80%, 70% to 75%, 80% to 100%, 80% to 99%, 80% to 95%, 80% to 90%, 80% to 85%, 90% to 100%, 90% to 99%, or 90% to 95%, including or excluding any foregoing numbers. All numbers can be modified by “about,” which is defined herein.

### Definition

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The practice of the present disclosure will employ, unless otherwise indicated, conventional techniques of microbiology, tissue culture, molecular biology, chemistry, biochemistry, and recombinant DNA technology, which are within the skill of the art. The materials, methods, and examples are illustrative only and not limiting. The following is presented by way of illustration and is not intended to limit the scope of the disclosure.

Numbers expressing quantities of ingredients, properties such as homogeneity, enrich level, molecular weight, reaction conditions and results, and so forth, used to describe and claim certain embodiments of the present disclosure are to be understood as being modified in some instances by the term “about.” A skilled artisan in the field would understand the meaning of the term “about” in the context of the value that it qualifies. The numerical values presented in some embodiments of the present disclosure may contain certain errors resulting from the standard deviation in their respective testing measurements. For example, the term “about,” as used herein, refers to a measurable value such as an amount, a temporal duration, and the like and is meant to encompass variations of ±5%, ±1%, or ±0.1% from the specified value, as such variations are appropriate.

As used herein, “substantially” means sufficient to work for the intended purpose. The term “substantially” thus allows for minor, insignificant variations from an absolute or perfect state, dimension, measurement, result, or the like, such as expected by a person of ordinary skill in the field, but that does not appreciably affect overall performance. When used with respect to numerical values or parameters or characteristics expressed as numerical values, “substantially” means within ten percent.

As used herein, “treat,” “treatment,” and “treating” refer to an approach for obtaining beneficial or desired results, for example, clinical results. For this disclosure, beneficial or desired results may include inhibiting or suppressing the initiation or progression of an infection or a disease; ameliorating, or reducing the development of, symptoms of an infection or disease; or a combination thereof.

As used herein, “preventing” and “prevention” are used interchangeably with “prophylaxis” and can mean complete prevention of infection or prevention of the development of symptoms of that infection, a delay in the onset of a disease or its symptoms; or a decrease in the severity of a subsequently developed infection or its symptoms.

As used herein, “glycan” refers to a polysaccharide, oligosaccharide, or monosaccharide. Glycans can be monomers or polymers of sugar residues and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.).

As used herein, a “recombinant glycoprotein” or a “recombinant protein” is a protein produced by introducing an engineered nucleic acid into a host organism, like bacteria, yeast, or mammalian cells using laboratory or industrial processes, and in some situations, isolated or purified for clinical or industrial uses.

### EXAMPLE

### Example 1: Manufacturing Exemplary CHO Cells of the Present Disclosure for Expressing a Sialylated Glycoprotein

This experiment manufactured an exemplary cell according to an embodiment of the present disclosure from a parental CHO cell. EXPICHO® cells (Thermo Fisher Scientific) were purchased and maintained EXPICHO®™ Expression Medium according to the product manual. Then, the cells were transferred with a SWG-006 plasmid (i.e. an expression vector) using an ExpiFectamine™ CHO Transfection Kit according to the manufacturer's instructions. The SWG-006 plasmid (SEQ ID NO: 23) was made of pcDNA™ 3.1 plasmid and was constructed with a CMV enhancer, a CMV promoter, a ST6GAL1 gene (SEQ ID NO: 10), a P2A sequence (SEQ ID NO: 19), and a B4GALT1 gene (SEQ ID NO: 14). Alternatively, cells were co-transfected with a ST6GAL1 plasmid (SEQ ID NO: 25) and a B4GALT1 plasmid (SEQ ID NO: 26), each was constructed to express the ST6GAL1 gene and the B4GALT1 gene separately under a CMV promoter.

Then, the cells were transfected with an Adalimumab plasmid (pcDNA2 TADA, SEQ ID NO: 27) using the ExpiFectamine™ CHO Transfection Kit to express Adalimumab (Humira). Three days after the transfection, the transfected cells (a transient clone) were washed with staining buffer (EDTA 1 mM, NaN₃ 0.02%, BSA 1%, in PBS) and stained with biotinylated SNA (VectorLabs), which is designed to bind a sialic acid linked to a N-acetylgalactosamine or galactose. After being washed, the cells were stained with Streptoavidin-Alexa647 (BioLegend) and analyzed using flow cytometry (BD LSRFortessa).

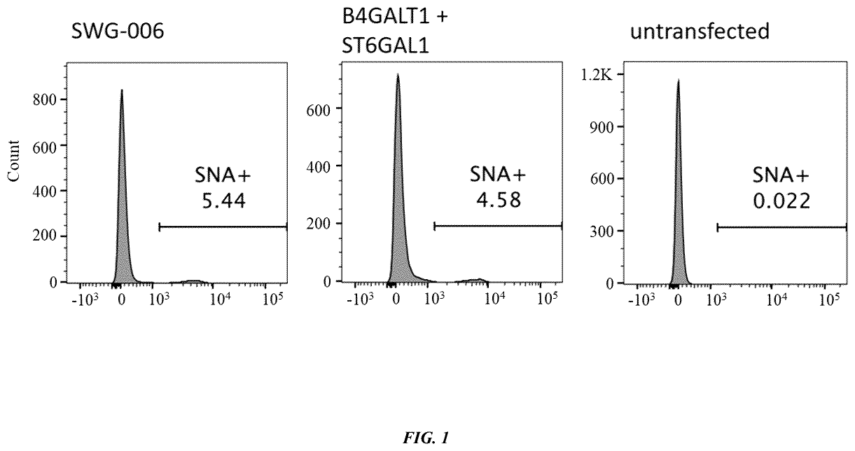

The results (FIG. 1) showed transfection with the SWG-006 plasmid or co-transfected with the ST6GAL1 plasmid and the B4GALT1 plasmid were able to promote α2,6-sialylation. In comparison, the parent CHO cell (EXPICHO® cells) does not have an endogenous α2,6-sialyltransferase, so no signal was detected from untransfected cells. Most importantly, the results also showed that transfection with the SWG-006 plasmid provided higher α2,6-sialylation than co-transfection with the ST6GAL1 plasmid and the B4GALT1 plasmid.

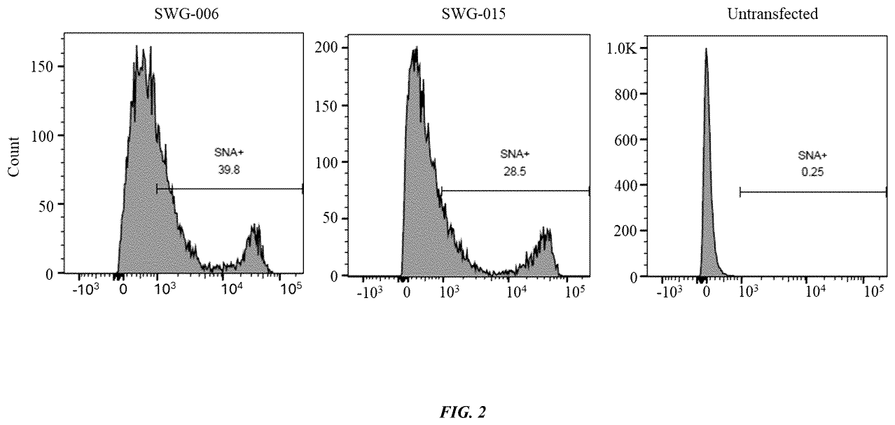

Similarly, another exemplary cell according to an embodiment of the present disclosure was made by transfecting an EXPICHO® cell (Thermo Fisher Scientific) using the ExpiFectamine™ CHO Transfection Kit with a SWG-015 plasmid (SEQ ID NO: 24). The SWG-015 vector wad made of pcDNA™ 3.1 plasmid and was constructed with a CMV enhancer, a CMV promoter, a modified PspST gene (SEQ ID NO: 12), a P2A sequence (SEQ ID NO: 19), and a B4GALT1 gene (SEQ ID NO: 14). The cell was also transfected with the Adalimumab plasmid (pcDNA2 TADA, SEQ ID NO: 27) to express Adalimumab (Humira). Then, the sialylation of Adalimumab produced by the cell, which is also a transient clone, was observed using SNA staining as described above. The cell transfected with the SWG-006 vector was also examined to compare with the cell transfected with the SWG-015 vector. The results showed that the cell transfected with the SWG-015 provided a similar level of sialylation as that of the cell transfected with the SWG-006 vector (FIG. 2).

### Example 2: Stabilization of the Exemplary Cells of the Present Disclosure

Plasmid SWG-006 was linearized by digestion with PvuI and then purification. The linearized SWG-006 was transfected into EXPICHO® cells by using ExpiFectamine™ CHO Transfection Kit (Thermo Fisher Scientific) according to the manufacturer's instructions. On day 2 post-transfection, G418 was added at 400 μg/ml for drug selection. After recovering to 90% survival, the cells were stained with biotinylated SNA and then streptavidin-BV421 wherein the SNA bound endogenous membrane proteins produced by the cells. The cells were then sorted for high SNA binding by using WOLF G2® Cell Sorter (Nanocellect Biomedical, Inc.) into single cells cultured in 96 well plates. After a period of time of culture, the single clone cells were stained with biotinylated SNA and then streptavidin-APC and analyzed by using flow cytometry to select clones with highly surface alpha2,6-sialylation. The selected cells (>99% SNA positive) were then transfected with Adalimumab expression plasmid (SEQ ID NO: 27) to overexpress Adalimumab. The expressed Adalimumab was purified by using protein A beads and analyzed by using an SNA hybridization blot to select clones that were able to express highly alpha2,6-sialylation Adalimumab. The glycoform of Adalimumab was further analyzed by LC/MS-MS.

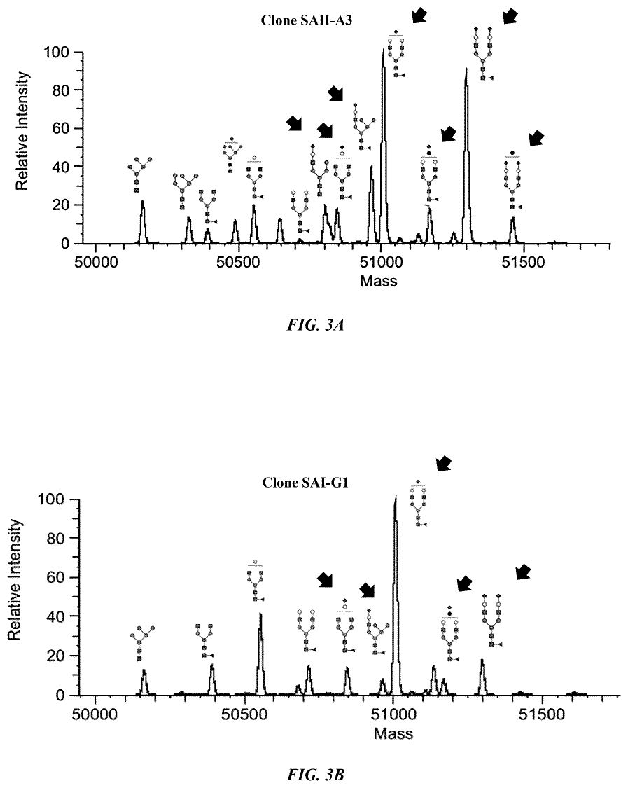

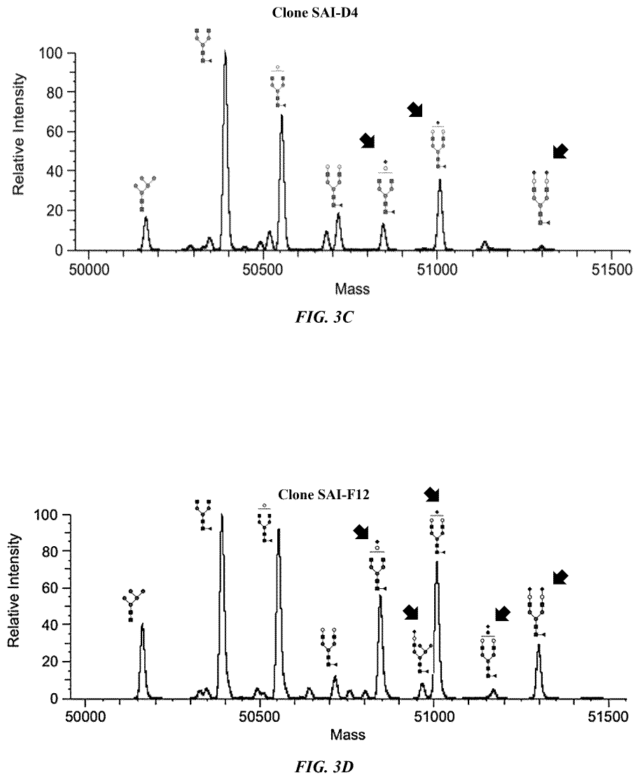

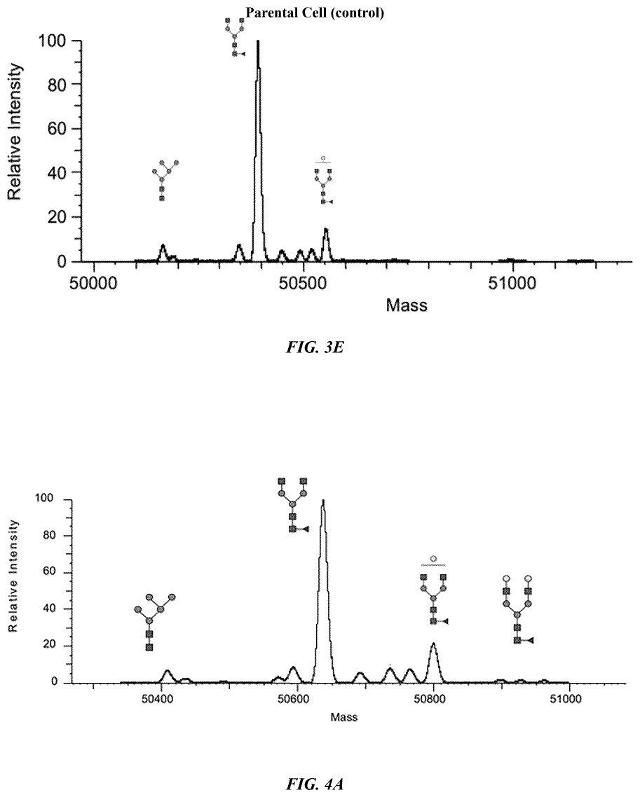

The LC/MS-MS data shows that the four stabilized clones tested in this example, SAII-A3, SAI-GI, SAI-D4, and SAI-F12, all expressed a high percentage of sialylation of Adalimumab. The stabilized Clone SAII-A3 showed 70.9% (about 70% to 72%) sialylation of the Adalimumab it expressed (FIG. 3A); the stabilized Clone SAI-GI showed 55.39% (about 55% to 57%) sialylation of the Adalimumab it expressed (FIG. 3B); the stabilized Clone SAI-D4 showed 16.8% (about 16% to 18%) sialylation of the Adalimumab it expressed (FIG. 3C); and the stabilized Clone SAI-F12 showed 36.14% (about 36% to 38%) sialylation of the Adalimumab it expressed (FIG. 3D). In comparison, the Adalimumab expressed by the parental cell did not show significant sialylation (FIG. 3E).

### Example 3: Glycoengineering Adalimumab Using an Exemplary Stabilized Cell According to the Present Disclosure

In this experiment, a stabilized clone (SAII-A3) of the SWG-006 cells obtained as described in Example 2 above was transfected with an Adalimumab expression vector (SEQ ID NO: 27) using an ExpiFectamine™ CHO Transfection Kit (Thermo Fisher Scientific) according to the manufacturer's instructions to express Adalimumab, and the glycoform thereof was observed and compared with the same antibody expressed by a parent cell (EXPICHO® cell).

Briefly, cells were diluted to a final density of 5×10\hat{ }6 viable cells/mL with fresh 15 mL EXPICHO®™ Expression Medium in a flask, pre-warmed to 37° C. The flask was then swirled gently. The cells were then maintained in a 37° C. incubator with a humidified atmosphere of 8% CO₂ in the air on a 125 rpm orbital shaker before transfection. Plasmid DNA (i.e., the expression vector of SEQ ID NO: 27, 12 μg) was diluted with 0.6 mL of cold OptiPRO™ medium, and ExpiFectamine™ CHO Reagent (48 μL) was diluted with 552 μL of cold OptiPRO™ medium. The dilated DNA was then mixed with the diluted ExpiFectamine™ CHO Reagent to obtain ExpiFectamine™ CHO/plasmid DNA complexes. The ExpiFectamine™ CHO/plasmid DNA complexes were added to the cell culture in a 37° C. incubator with a humidified atmosphere of 8% CO₂ in the air on a 125 rpm orbital shaker. After transfection, 90 μl ExpiFectamine™ CHO Enhancer and 3.6 ml EXPICHO®™ Feed volumes were added to the cells. The cells in the flask were then maintained in the 37° C. incubator with a humidified atmosphere of 8% CO₂ with shaking. After 8-10 days post-transfection, the supernatant of the cell culture was harvested for protein purification.

Then, the antibodies produced by the SAII-A3 cells and the parental EXPICHO® cells were isolated respectively using protein A beads (GE HealthCare). Briefly, 15 mL supernatant was passed through the 200 μL protein A column. The column was washed with 10-column volume (CV) PBS. The bound antibody was eluted with 15 CV 0.1 M glycine pH=3. The yielding antibodies were dialysis against 10 mM ammonium acetate. The antibodies were then reduced using 25 mM DTT and analyzed by LC/MS-MS. A commercial product of Adalimumab (Humira®) was purchased and analyzed using LC/MS-MS as a control.

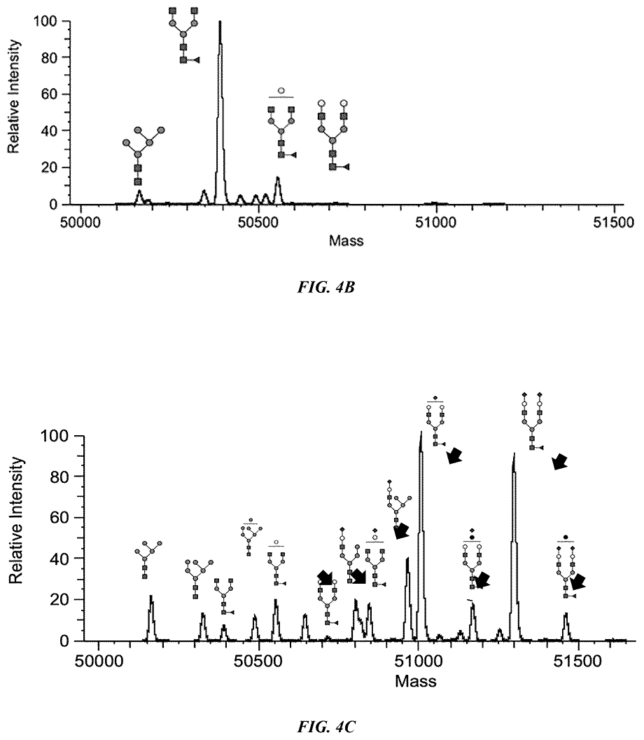

The LC/MS-MS data shows that the Adalimumab produced by the exemplary cells of the present disclosure had a variety of sialylated glycans (See FIG. 4C), the sialylated glycans are labeled with solid arrows), including G2S2F, G2S1F, G1S1F, N3H6F1S1, N3H4S1, N3H5F1S1, N4H6F1S1, N4H6F1S2. The sialylation percentage of the total glycans was 70.9%, and the sialylation percentage of the bi-antennary complex type glycan was 79.8%. In comparison, although the commercial products (FIG. 4A) and the Adalimumab produced by parental EXPICHO® cells (FIG. 4B) also carried glycans, no sialylation was observed.

### Example 4: Manufacturing Exemplary HEK293 Cells of the Present Disclosure for Expressing a Sialylated Glycoprotein

This experiment manufactured an exemplary cell according to an embodiment of the present disclosure from a parental HEK293T cell using CRISPR technologies. The results were very similar to the results of using the ExpiFectamine™ CHO Transfection Kit on CHO cells, as described in Example 1.

Cells, Plasmids, and Transfection. The parental HEK293T cells (ATCC) were cultured in the DMEM (Thermo Fisher Scientific) supplied with 10% FBS (Thermo Fisher Scientific) at 37° C. with 5% CO₂ in the cell incubator. The plasmids for CRISPR/Cas9 expression were constructed as suggested by the kit manual (Thermo Fisher Scientific). Briefly, the validated sgRNA sequence targeting glycosyltransferase was synthesized (Integrated DNA Technologies) and cloned into the vector for Cas9 and sgRNA expression. The synthetic codon-optimized gene insert (Integrated DNA Technologies), flanked by the homologous arm of the target gene at the sgRNA target site, was cloned into an empty vector as the donor plasmid. Transfection of 293T cells was mediated by TransIT-293 (Mirus Bio) following the reagent manual. A plasmid for expressing antibody chMC81370 (a humanized antibody targeting specific stage embryonic antigen 4 (SSEA4)) was transfected into cells followed by incubation. At the time of harvest, the culture medium was collected and then subjected to protein A sepharose beads (GE HealthCare) column to purify the antibodies.

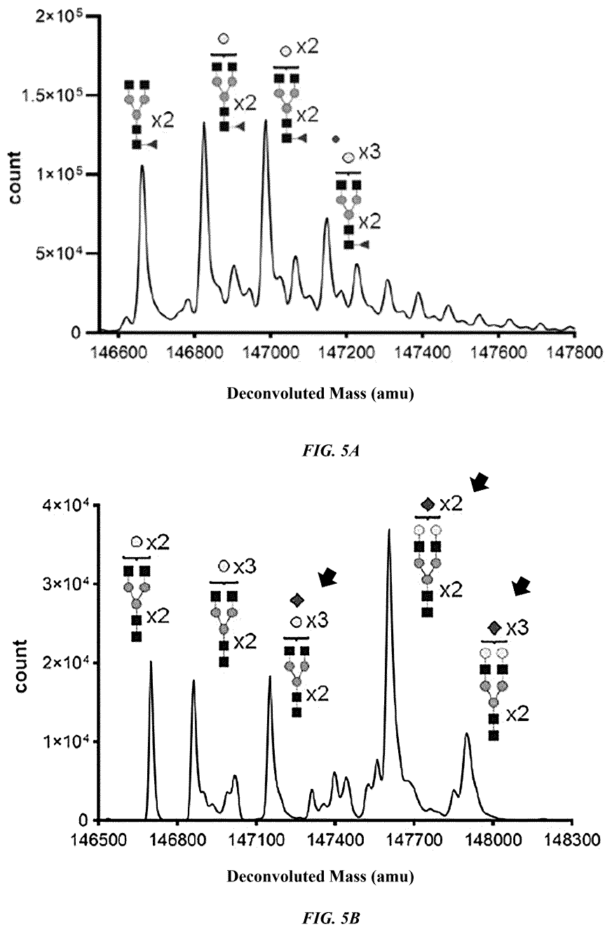

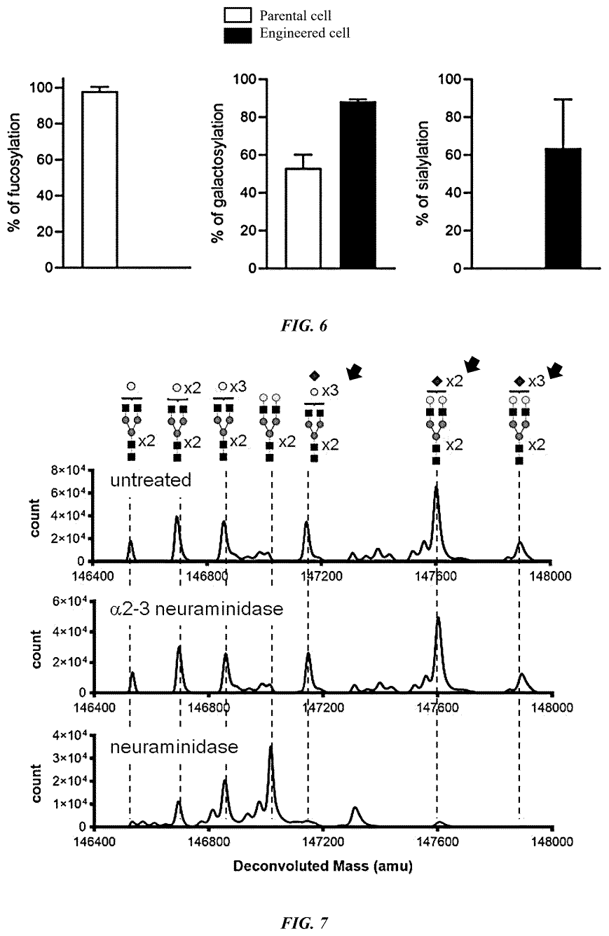

Analysis. The purified antibodies were analyzed using intact protein mass (IPM) analysis. Briefly, the samples were diluted with LCMS grade water at a concentration between 5-10 μM and analyzed by 6230 TOF LC/MS with a Dual AJS ESI ion source (Agilent Technologies) with PLRP-S 1000 Å 5 μm column (Agilent Technologies). Solvent A was 0.1% Formic Acid in H₂O and Solvent B was 0.1% Formic Acid in CAN. The results show a significant increase in galactosylated and sialylated antibody glycoforms (FIG. 5B) compared to the antibodies generated by the parental HEK293T cells (FIG. 5A). The results of the IPM Glycoform analysis were presented in bar charts, which revealed a significant increase in antibody galactosylation and sialylation, with a lack of core-fucosylation (FIG. 6).

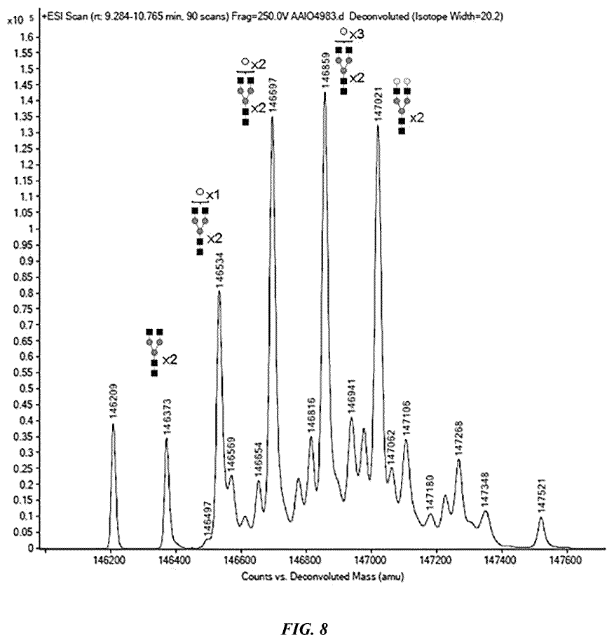

Characterizing the sialylation. To confirm the sialylation on the antibody was mediated by hST6Gal1, the antibody was treated with two different sialidases, Streptococcus pneumonia α2-3 neuraminidase and Clostridium perfringens neuraminidase at 37° C. overnight respectively followed by purification or gel electrophoresis. The intact protein analysis did not show any obvious difference in the distribution of asialylated and sialylated antibodies between the untreated and α2-3 neuraminidase-treated group (FIG. 7 upper and middle; sialylated glycoforms are labeled with the solid arrows), indicating that the sialylation on antibody was not due to the endogenous α2-3 sialyltransferases. On the other hand, treatment with Clostridium perfringens neuraminidase, which could remove all linkages of sialylation, largely eliminated the terminal sialic acids (FIG. 7 bottom). In addition, the antibodies with increased galactosylation generated from cells with knock-in of hB4GalT1 only did not show terminal sialylation (FIG. 8). These results confirmed that sialylation on the antibody was introduced by hST6Gal1 and the increase of hB4Gal1 expression indeed elevated the galactosylation of antibody (FIG. 5).

The sialyltransferase hST6Gal1 is known as the only human glycosyltransferase that mediates α2-6 sialylation and preferentially uses the α1-3 branch arm of the biantennary glycan as substrates. Some in vitro studies show glycoengineering on antibody glycans by hST6Gal1 takes a long time and is difficult to yield fully sialylated antibody glycans. Furthermore, this also could explain why only the antibody with at least one fully galactosylated glycan was sialylated. If the galactose on G1 glycan was not on the al-3 branch arm, the hST6Gal1 may not sialylate it. Therefore, the expression level of glycosyltransferases may not be the bottleneck in this situation. Rather, substrate preference and enzyme activity are the major factors. Using α2-6 sialyltransferases from other organisms may have similar or better activity than hST6Gal1, which is evident by the other examples described herein that used a PspST gene.

### Example 5: Time Factor in Glycoengineering Using an Exemplary Cell of the Present Disclosure

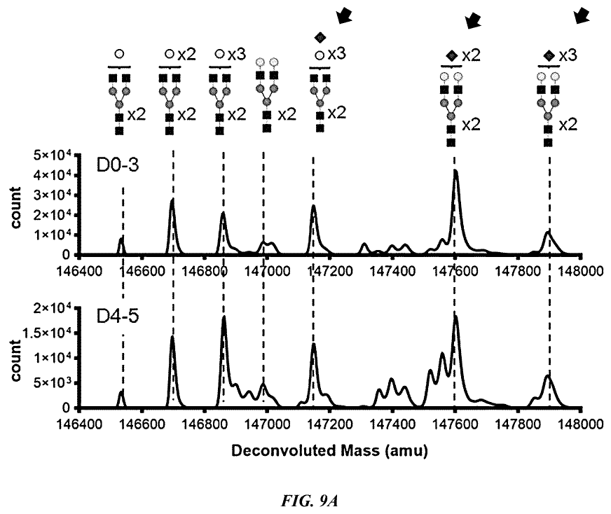

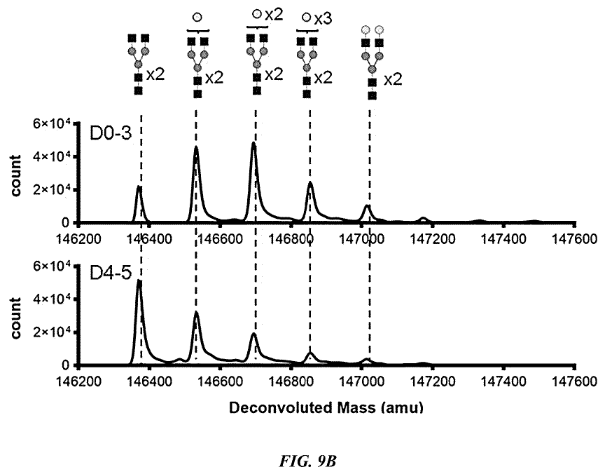

This experiment tested whether the amount and the time of antibody expression using an exemplary cell of the present disclosure affect glycosylation. The culture time was extended to 5 days, and the medium of the culture was collected on Day 3 and Day 5, respectively. The produced antibodies were examined using intact protein mass (IPM) analysis. The results show a relatively strong sialylation in the first collection (first three days) (FIG. 9A, D0-3), and then sialylation was decreased in the second collection (last two days), accompanied by an increase of terminal galactosylation (FIG. 9A, D4-5), suggesting that the proteins involved in glycosylation may not be sufficient to effectively produce Fc-SCT antibody at a later stage.

Because the hST6Gal1 and hB4GalT1 involved in the FC-SCT antibody production were almost artificially and constitutively expressed in the cells, we also examined if endogenous glycosyltransferases faced the same problem of reduced glycosylation. By expressing antibodies in FUT8 KO cells, the antibody was almost terminally glycosylated with at least one or more galactoses in the first collection (FIG. 9B, D0-3), but the proportion of galactosylated antibody decreased, and even the antibody glycans without any galactoses became the dominant glycoforms in the last two days ((FIG. 9B, D4-5). The result showed that the dynamic glycosylation level after the antibody expression can be observed on endogenous glycosylation products as well.

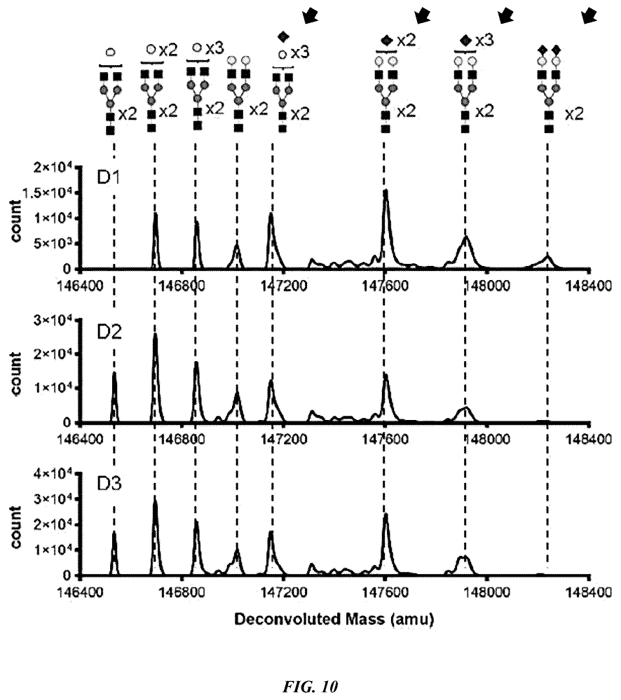

The aforementioned finding suggests that antibody production at different time points may impact glycosylation differently. Therefore, in the next experiment, antibodies were purified every day after plasmid transfection and analyzed the dynamic change of antibody glycans from multiple engineered cells. The antibody with full sialylation could be detected by intact protein mass analysis on the first day and then gradually decreased over time (FIG. 10). Regarding galactosylation, the antibody glycans were capped by at least two galactoses (2˜4 galactoses in total) on the first day, but started to decline on the second day, as evidenced by the appearance of one galactose on antibody glycans (FIG. 10). These observations suggest that antibody glycosylation was almost complete at the beginning of the protein expression and gradually decreased over time.

### Example 6: Binding Between Sialylated Antibodies and Fcγ Receptors

FcγRIIA (Fc gamma RIIA) and FcγRIIB (Fc gamma RIIB) are usually expressed simultaneously by the antigen-presenting cells, such as dendritic cells and macrophages, and these two receptors work together to modulate immune responses. FcγRIIIA is critical for NK cells to mediate ADCC3. This experiment tested whether the Fc-SCT-enriched antibodies produced using the exemplary cells of the present disclosure show better binding affinity than wide-type antibodies (i.e., antibodies produced by parental cells). The Fc-SCT-enriched antibodies were produced and purified as described in Example 4. The binding affinity was examined using an ELISA assay.

ELISA. The recombinant soluble FcγIIA, FcγIIB, and FcγRIIIA (Fc gamma RIIIA, R&D) was coated with 50 ng/well in bicarbonate/carbonate coating buffer (50 mM, pH 10) at 4° C. overnight before blocking the well with 5% BSA in TPBS (0.05% Tween 20 in PBS) at 4° C. overnight. The antibody was added into wells with final concentration started at 100 μg/ml with 5× series dilution and incubated at room temperature for 1 hour followed by incubation with HRP-conjugated goat anti-human IgG antibody (Jackson ImmunoResearch) for another 1 hour at room temperature. Finally, the TMB substrate (Bethyl Laboratories) was added to react with HRP at RT prior to stopping the reaction by H₂SO₄. The absorbance at 450 nm was detected by SPECTRAMAX® M5 spectrum reader (Molecular Device). The wells were washed by TPBS 3-5 times between each step.

The results show that the binding of Fc-SCT-enriched antibodies to FcRs was increased in three tested Fc receptors (FIG. 11). Among these receptors, the increased binding of FcγRIIA and FcγRIIIA was consistent with previous studies. Interestingly, the WT antibody showed very weak binding to the inhibitory receptor FcγRIIB. Despite the improvement being less significant than that of FcγRIIA and FcγRIIIA, the glycoengineering by the cells of the present disclosure was able to improve the antibody's binding to receptor FcγRIIB. Although the increased binding to the two opposing functional receptors, FcγRIIA and FcγRIIB, which are typically expressed on the antigen-presenting cells (APCs) simultaneously, appears to be conflicting, the eventual immune response was determined by the expression level and signaling strength of FcγRIIA and FcγRIIB on effector cells. The Fc-SCT-enriched antibody also exhibited stronger FcγRIIIA binding ability than the antibody produced from wild-type cells (FIG. 11). Without wishing to be bound by theories, this enhancement was also contributed by the removal of core-fucose11 and the addition of galactose.

### Example 7: Manufacturing Exemplary HEK293 Cells of the Present Disclosure for Expressing GlcNAc Glycoproteins

This experiment manufactured an exemplary cell according to an embodiment of the present disclosure from a parental GnTI KO ExpiHEK293F cell using CRISPR technologies. The parental cells were prepared as described in Example 4. The N-glycan expressed by this cell line was a high mannose type which was preferentially cleaved by Endo H to generate Fc-GlcNAc antibody. Therefore, this experiment aims to knock in Endo H (UniProtKB/Swiss-Prot: P04067.1, Sec SEQ ID NO: 15 and SEQ ID NO: 17) in this cell line by CRISPR-Cas9. The plasmid for the CRISPR technology was prepared as described below.

Plasmids and Transfection. The plasmids for CRISPR/Cas9 expression were constructed as suggested by the kit manual (Thermo Fisher Scientific). Briefly, the validated sgRNA sequence targeting glycosyltransferase was synthesized (Integrated DNA Technologies) and cloned into the vector for Cas9 and sgRNA expression. The synthetic codon-optimized gene insert (Integrated DNA Technologies), flanked by the homologous arm of the target gene at the sgRNA target site, was cloned into an empty vector as the donor plasmid. Transfection of 293T cells was mediated by using an Expi293Fectamine™ Transfection Kit (Thermo Fisher Scientific) following the reagent manual. A plasmid for expressing antibody chMC81370 was transfected into cells followed by incubation. At the time of harvest, the culture medium was collected and then subjected to protein A sepharose beads (GE HealthCare) column to purify the antibody.

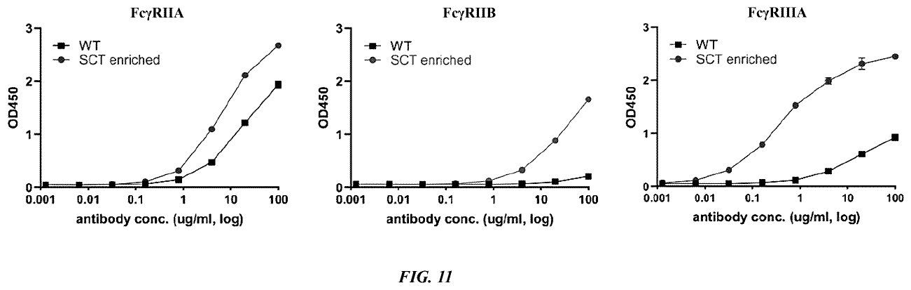

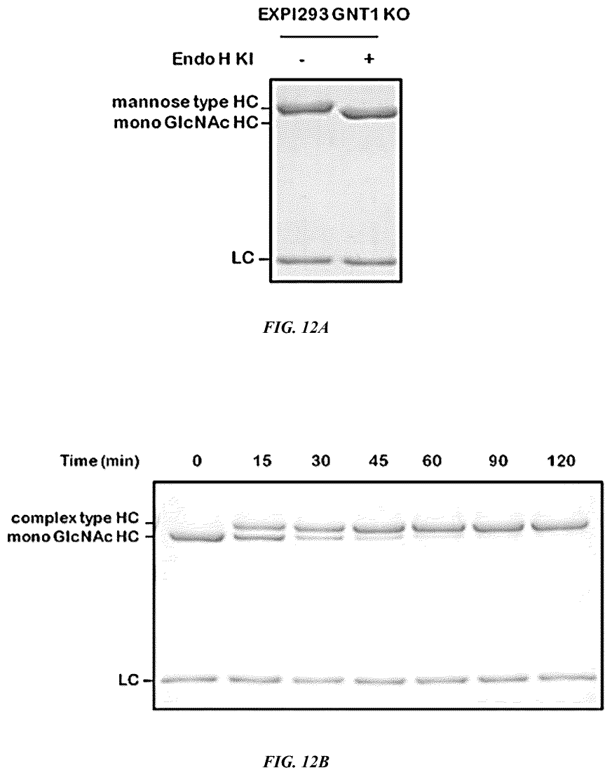

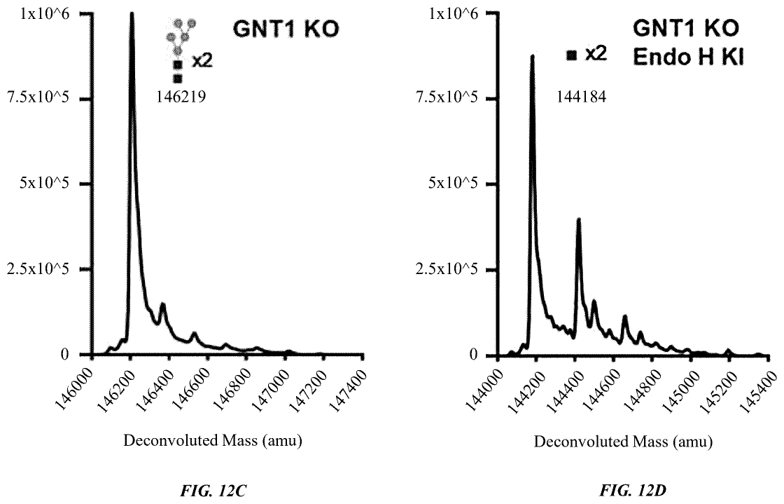

Analysis. The antibodies expressed by the cells were analyzed by electrophoresis. The protein was heated at 95° C. for 5 mins in the LDS sample buffer supplied with 2-Mercaptoethanol and then subjected to gel electrophoresis with 12% SDS-PAGE. The protein on the gel was stained by Coomassie blue buffer (ApexBio). The result showed that the antibody heavy chain (AbHC) expressed by these cells had faster mobility than AbHC from its parental cells (GnT1 KO), and most AbHCs were downshifted (FIG. 12A).

This antibody was also subjected to a transglycosylation assay. The antibody was incubated with SCT glycan-oxazoline and Endo S2 mutant in Tris buffer at 37° C. for the time indicated before harvest for purification or gel electrophoresis. The transglycosylation generated antibodies with homogenous glycan (in this experiment, homogenous SCT glycan). The efficiency of the transglycosylation was pretty good (FIG. 12B). The glycans on these antibodies were then by intact protein mass (IPM) analysis. The results showed that most antibodies (over 95%) contain only GlcNAc on each HC (FIG. 12C, FIG. 12D, and FIG. 12E).

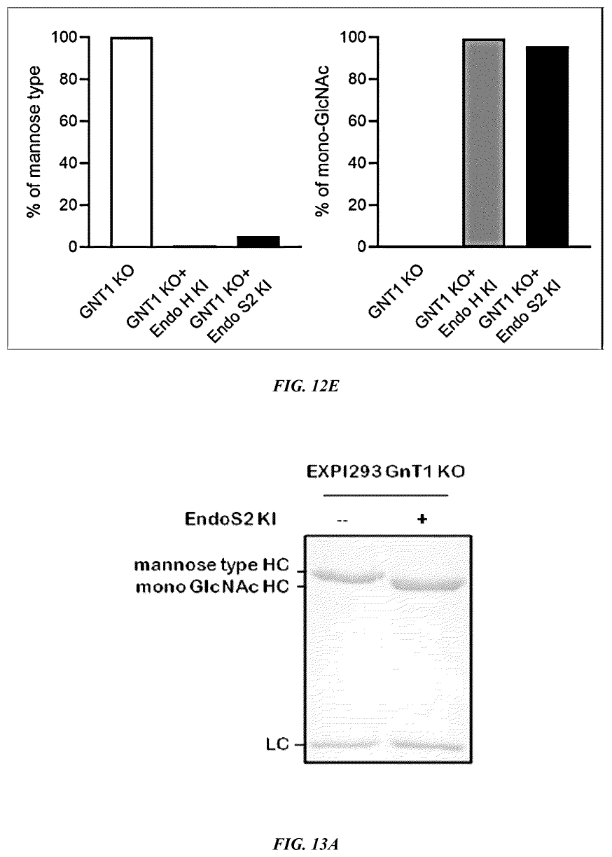

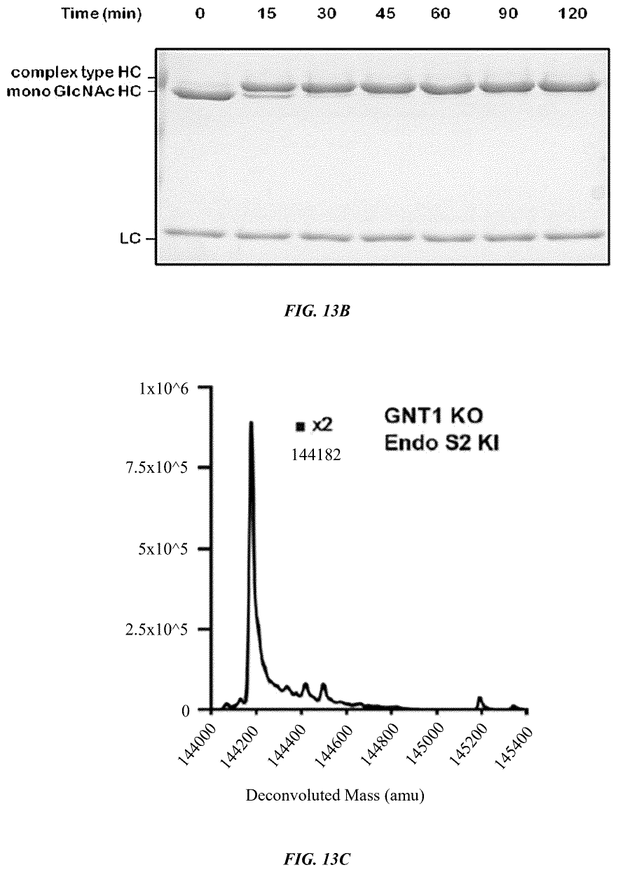

Alternatively, instead of knocking in Endo H, endoglycosidase S2 (Endo S2; UniProtKB/Swiss-Prot: T1WGN1.1, See SEQ ID NO: 16 and SEQ ID NO: 18) from Streptococcus pyogenes was knocked in because this enzyme can hydrolyze both high mannose and complex type N-glycans to Fc-GlcNAc. The donor vector and CRISPR-Cas9 plasmids were delivered to GnT1 KO cells. Then the antibody glycans generated by these cells were examined as previously described. The protein gel showed an obvious downshifted band of AbHC compared with its parental cells (GNT1 KO) (FIG. 13A). Besides, the glycans also can be added onto AbHC through enzymatic transglycosylation (FIG. 13B) and intact protein mass analysis showed a clear signal of Fc-GlcNAc antibody (FIG. 13C), most antibodies (over 90%) contain only GlcNAc on each HC (FIG. 12E).

Based on these results, it is concluded that both Endo H and Endo S2 can be introduced to cells by CRISPR-Cas9 to process the N-glycans to Fc-GlcNAc anti-body for in vitro transglycosylation. In addition, this cell-based method can be used for other glycoproteins, such as influenza hemagglutinin and SARS-COV-2 spike protein to generate the mono-GlcNAc decorated glycoforms as vaccines to elicit broadly protective immune responses

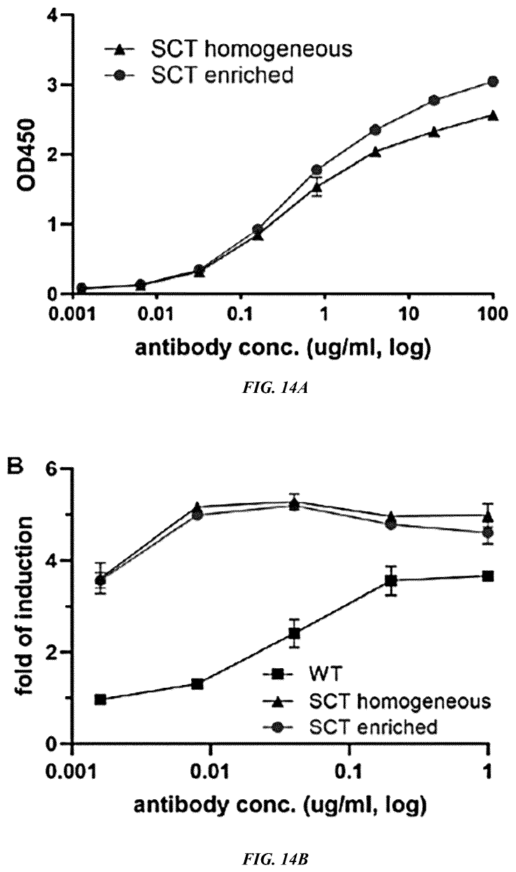

Next, since the binding of Fc-SCT enriched antibodies (i.e., the antibody obtained in Example 4) to FcγIIIA receptor was elevated, the homogeneous Fc-SCT antibodies obtained above through transglycosylation were compared with the Fc-SCT enriched antibodies for their binding affinity to FcγIIIA receptors. The result showed that Fc-SCT enriched antibodies exhibited similar binding avidity to homogenous SCT antibodies (FIG. 14A).

Furthermore, an ADCC reporter assay was conducted. Briefly, the assay was performed according to the kit manual. The SSEA4 expressing target cells SK-OV3 were seeded into a 96-well plate, followed by the addition of an antibody with the final concentration started at 1 g/ml with 5×series dilution. Then, the FcγRIIIA expressing effector cells (effector: target cell ratio, 6:1) were added and incubated with target cells in the incubator at 37° C. for 6 hrs. The plate was placed at room temperature for 15 min prior to adding the luciferase substrate. After 5 min incubation, the luminescence was measured by SPECTRAMAX® M5 spectrum reader. The induction fold was calculated by RLU (induced−background)/RLU (no antibody control−background). It was observed that at the lowest antibody concentration used for induction, WT antibody did not induce any obvious cell activation, but the homogenous Fc-SCT and Fc-SCT enriched antibodies can trigger cell activation and rapidly reached a maximum at next concentration (FIG. 14B). Even at the higher concentration, the WT antibody can reach a maximum induction, but the activity was still much lower than that of homogenous Fc-SCT or Fc-SCT enriched antibody (FIG. 14B).

### EXEMPLARY EMBODIMENTS

Embodiment 1: A cell for expressing a sialylated glycoprotein wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity.

Embodiment 2: The cell of Embodiment 1, wherein the exogenous sialyltransferase catalytic peptide and the exogenous galactosyltransferase catalytic peptide are expressed in a single transcript.

Embodiment 3: The cell of Embodiment 1 or Embodiment 2, comprising a first nucleic acid configured to express the exogenous sialyltransferase catalytic peptide and a second nucleic acid configured to express the exogenous galactosyltransferase catalytic peptide, wherein the first nucleic and the second nucleic acid are transcriptionally controlled by the same promoter.

Embodiment 4: The cell of Embodiment 3, wherein the first nucleic acid and the second nucleic acid are connected to each other via a connecting nucleic acid, which is configured to encode a ribosomal shifting peptide.

Embodiment 5: The cell of Embodiment 4, wherein the ribosomal shifting peptide comprises an amino acid sequence of DxExNPGP, wherein x denotes any amino acid, D denotes aspartic acid, E denotes glutamic acid, N denotes asparagine, P denotes proline, and G denotes glycine.

Embodiment 6: The cell of Embodiment 4 or Embodiment 5, wherein the ribosomal shifting peptide comprises an amino acid sequence as set forth in SEQ ID NO: 06, SEQ ID NO: 07, SEQ ID NO: 08, or SEQ ID NO: 09.

Embodiment 7: The cell of Embodiment 3, wherein the exogenous sialyltransferase catalytic peptide and the exogenous galactosyltransferase catalytic peptide are co-configured to be expressed as a fusion protein.

Embodiment 8: The cell of Embodiment 7, wherein the fusion protein comprises a first portion having the sialyltransferase catalytic peptide domain and a second portion having the galactosyltransferase catalytic peptide domain, wherein the first portion and the second portion are connected to each other via a cleavable linker, further wherein the cleavable linker is configured to be cleavable post-translation of the fusion protein, thereby upon cleavage releasing the sialyltransferase catalytic peptide and the galactosyltransferase catalytic peptide as separate proteins.

Embodiment 9: The cell of any one of Embodiments 3 to 8, wherein the first nucleic acid is derived from an ST6Gal1 gene or a PspST gene.

Embodiment 10: The cell of any one of Embodiments 3 to 9, wherein the first nucleic acid comprises a nucleotide sequence as set forth in SEQ ID NO: 10 or SEQ ID NO: 11.

Embodiment 11: The cell of Embodiment 10, wherein the first nucleic acid comprises a nucleotide sequence as set forth in SEQ ID NO: 12 or SEQ ID NO: 13.

Embodiment 12: The cell of any one of Embodiments 3 to 11, wherein the second nucleic acid is derived from an B4GALT1 gene.

Embodiment 13: The cell of any one of Embodiments 3 to 12, wherein the second nucleic acid comprises a nucleotide sequence as set forth in SEQ ID NO: 14.

Embodiment 14: The cell of any one of Embodiments 3 to 13, wherein the promoter is a constitutive promoter or an activable promoter.

Embodiment 15: The cell of Embodiment 14, wherein the constitutive promoter is a CMV promoter, T7 promoter, a Human Elongation Factor 1 Alpha (EF1α) promoter, a Chicken β-Actin (CAG) promoter, or an SV40 promoter.

Embodiment 16: The cell of Embodiment 14, wherein the activable promoter is a Tetracycline-Inducible Promoter (activable by doxycycline) or a dihydrofolate reductase (DHFR) gene promoter.

Embodiment 17: The cell of any one of Embodiments 1 to 16, wherein the sialyltransferase catalytic peptide is an alpha-2,6-sialyltransferase.

Embodiment 18: The cell of Embodiment 17, wherein the sialyltransferase is a beta-galactoside alpha-2,6-sialyltransferase 1.

Embodiment 19: The cell of any one of Embodiments 1 to 18, wherein the sialyltransferase catalytic peptide comprises an amino acid sequence as set forth in SEQ ID NO: 01 or SEQ ID NO: 02.

Embodiment 20: The cell of Embodiment 19, wherein the sialyltransferase catalytic peptide comprises an amino acid sequence as set forth in SEQ ID NO: 03 or SEQ ID NO: 04.

Embodiment 21: The cell of any one of Embodiments 1 to 20, wherein the galactosyltransferase catalytic peptide is a beta-1,4-galactosyltransferase 1.

Embodiment 22: The cell of Embodiment 21, wherein the galactosyltransferase catalytic peptide comprises an amino acid sequence as set forth in SEQ ID NO: 05.

Embodiment 23: The cell of any one of Embodiments 1 to 22, wherein the cell is deficient in fucosyltransferase activity.

Embodiment 24: The cell of Embodiment 23, wherein the cell is deficient in fucosyltransferase 8 activity.

Embodiment 25: The cell of Embodiment 23 or Embodiment 24, wherein the cell is deficient in a FUT8 gene encoding a product of the fucosyltransferase activity.

Embodiment 26: The cell of any one of Embodiments 1 to 25, wherein the cell is a mammalian cell.

Embodiment 27: The cell of Embodiment 26, wherein the cell is derived from a Chinese hamster ovary cell or a HEK293 cell.

Embodiment 28: The cell of any one of Embodiments 1 to 27, wherein the cell further comprises a payload nucleic acid configured to encode a recombinant glycoprotein, and the expression of the payload nucleic acid is transcriptionally controlled by a constitutive or an activable promoter.

Embodiment 29: The cell of Embodiment 28, wherein the recombinant glycoprotein is an antibody or an antigen-binding fragment thereof.

Embodiment 30: The cell of Embodiment 29, wherein the antibody is a therapeutic antibody.

Embodiment 31: A method for glycoengineering a recombinant glycoprotein comprising: delivering an expression vector into a cell according to any one of Embodiments 1 to 27, wherein the expression vector comprises a payload nucleic acid configured to encode the recombinant glycoprotein; and expressing the payload nucleic acid in the cell, thereby obtaining a plurality of recombinant glycoproteins, where at least one recombinant glycoprotein of the plurality is conjugated with a sialylated glycan.

Embodiment 32: The method of Embodiment 31, wherein the sialylated glycan is an α2-6 sialyl complex type (SCT) glycan.

Embodiment 33: The method of Embodiment 32, wherein the α2-6 sialyl complex type (SCT) glycan is mono-antennary or bi-antennary.

Embodiment 34: The method of Embodiment 31 or Embodiment 32, wherein the sialylated glycan lacks a core-fucose.

Embodiment 35: The method of any one of Embodiments 31 to 34, wherein the expression of the payload nucleic acid is constitutive or controllable.

Embodiment 36: The method of any one of Embodiments 31 to 35, wherein at least 50% of the plurality of the recombinant glycoproteins is conjugated with the sialylated glycan.

Embodiment 37: The method of any one of Embodiments 31 to 36, wherein the plurality of the recombinant glycoproteins is conjugated with the sialylated glycan at an enriched homogeneity of at least 10%, 15%, 20%, 25%, 30%, 35%, 40%, 45% 50%, 55%, 60%, 65%, 70%, 75%, 80%, 85%, 90%, 94%, 95%, 99%, 100% or an integer ranging between any of the above two numbers.

Embodiment 38: The method of any one of Embodiments 31 to 37, further comprising harvesting the plurality of recombinant glycoproteins within 200 hours from the expression of the payload nucleic acid in the cell.

Embodiment 39: The method of Embodiment 38, wherein the plurality of recombinant glycoproteins was harvested within 100 hours from the expression of the payload nucleic acid in the cell.

Embodiment 40: The method of any one of Embodiments 31 to 39, wherein the recombinant glycoprotein is an antibody or an antigen-binding fragment thereof.

Embodiment 41: The method of Embodiment 40, wherein the glycan is on a constant region of the antibody.

Embodiment 42: The method of Embodiment 41, wherein the glycan is on a heavy chain of the antibody.

Embodiment 43: The method of any one of Embodiments 40 to 42, wherein the glycan is on a glycosylation site of the antibody.

Embodiment 44: The method of Embodiment 43, wherein the glycan is on an N46, N402, and/or N297 site of the antibody.

Embodiment 45: The method of any one of Embodiments 40 to 44, wherein the antibody is a therapeutic antibody.

Embodiment 46: The method of Embodiment 45, wherein the therapeutic antibody is Adalimumab (Humira®), Adalimumab-atto (Amjevita®), Rituximab (Rituxan®), Rituximab-atto (Truxima®), Cetuximab (Erbitux®), Bevacizumab (Avastin®), Infliximab (Remicade®), Trastuzumab (Herceptin®), Pembrolizumab (Keytruda®), Etanercept (Enbrel®), Ipilimumab (Yervoy®), Ofatumumab (Arzerra), Golimumab (Simponi®), Atezolizumab (Tecentriq®), Ocrelizumab (OCREVUS®), Durvalumab (Durvalumab®), Avelumab (Bavencio®), Nivolumab (Opdivo®), Pertuzumab (Perjeta®), Obinutuzumab (Gazyva®), Gazyvaro Infliximab (Remicade®), or Trastuzumab emtansine (Kadcyla®).

Embodiment 47: A plurality of enriched recombinant glycoproteins, wherein at least 50% of the plurality of recombinant glycoproteins is configured with a sialylated glycan.

Embodiment 48: The plurality of enriched recombinant glycoproteins of Embodiment 47, wherein at least 70% of the plurality of enriched recombinant glycoproteins is configured with the sialylated glycan.

Embodiment 49: The plurality of enriched recombinant glycoproteins of Embodiment 47 or Embodiment 48, wherein the plurality of enriched recombinant glycoproteins is conjugated with the sialylated glycan at homogeneity of at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Embodiment 50: The plurality of enriched recombinant glycoproteins of Embodiment 47 or Embodiment 48, wherein the plurality of the enriched recombinant glycoproteins is conjugated with the sialylated glycan at homogeneity of 1% to 100%, 1% to 90%, 1% to 80%, 1% to 70%, 1% to 60%, 1% to 50%, 1% to 40%, 1% to 30%, 1% to 20%, 1% to 10%, 5% to 100%, 5% to 95%, 5% to 85%, 5% to 75%, 5% to 65%, 5% to 55%, 5% to 45%, 5% to 35%, 5% to 25%, 5% to 10%, 10% to 100%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 100%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 100%, 40% to 99%, 40% to 95%, 40% to 90%, 40% to 85%, 40% to 80%, 40% to 75%, 40% to 70%, 40% to 65%, 40% to 60%, 40% to 55%, 40% to 50%, 60% to 100%, 60% to 99%, 60% to 95%, 60% to 90%, 60% to 85%, 60% to 80%, 60% to 75%, 50% to 100%, 50% to 99%, 50% to 95%, 50% to 90%, 50% to 85%, 50% to 80%, 50% to 75%, 50% to 70%, 50% to 65%, 50% to 60%, 70% to 100%, 70% to 99%, 70% to 95%, 70% to 90%, 70% to 85%, 70% to 80%, 70% to 75%, 80% to 100%, 80% to 99%, 80% to 95%, 80% to 90%, 80% to 85%, 90% to 100%, 90% to 99%, or 90% to 95%.

Embodiment 51: The plurality of enriched recombinant glycoproteins of any one of Embodiments 47 to 50, wherein the sialylated glycan is an α2-6 sialyl complex type (SCT) glycan.

Embodiment 52: The plurality of enriched recombinant glycoproteins of Embodiment 51, wherein the α2-6 sialyl complex type (SCT) glycan is mono-antennary or bi-antennary.

Embodiment 53: The plurality of enriched recombinant glycoproteins of Embodiment 51 or Embodiment 52, wherein the sialylated glycan lacks a core-fucose.

Embodiment 54: The plurality of enriched recombinant glycoproteins of any one of Embodiments 47 to 53, wherein the recombinant glycoprotein is an antibody or an antigen-binding fragment thereof.

Embodiment 55: The plurality of enriched recombinant glycoproteins of Embodiment 54, wherein the glycan is on a constant region of the antibody.

Embodiment 56: The plurality of enriched recombinant glycoproteins of Embodiment 55, wherein the glycan is on a heavy chain of the antibody.

Embodiment 57: The plurality of enriched recombinant glycoproteins of any one of Embodiments 54 to 56, wherein the glycan is on a glycosylation site of the antibody.

Embodiment 58: The plurality of enriched recombinant glycoproteins of Embodiment 57, wherein the glycan is on an N46, N402, and/or N297 site of the antibody.

Embodiment 59: The plurality of enriched recombinant glycoproteins of any one of Embodiments 54 to 58, wherein the antibody is a therapeutic antibody.

Embodiment 60: The plurality of enriched recombinant glycoproteins of any one of Embodiments 47 to 59, being obtained by using the method for glycoengineering a recombinant glycoprotein of any one of Embodiments 31 to 46.

Embodiment 61: A cell for expressing a GlcNAc glycoprotein, being deficient in N-acetylglucosaminyltransferase I (GnTI) activity and constitutively or controllably expressing an exogenous endoglycosidase.

Embodiment 62: The cell of Embodiment 61, wherein the exogenous endoglycosidase is an endoglycosidase H (Endo H) or an endoglycosidase S2 (Endo S2).

Embodiment 63: The cell of Embodiment 62, wherein the exogenous endoglycosidase comprises an amino acid sequence as set forth in SEQ ID NO: 15 or SEQ ID NO: 16.

Embodiment 64: The cell of any one of Embodiments 61 to 63, wherein the cell comprises a nucleic acid configured to encode the exogenous endoglycosidase.

Embodiment 65: The cell of Embodiment 64, wherein the nucleic acid comprises a nucleotide sequence as set forth in SEQ ID NO: 17 or SEQ ID NO: 18.

Embodiment 66: The cell of any one of Embodiments 61 to 65, wherein the cell is a mammalian cell.

Embodiment 67: The cell of Embodiment 66, wherein the cell is derived from a Chinese hamster ovary cell or a HEK293 cell.

Embodiment 68: The cell of any one of Embodiments 61 to 67, wherein the cell is derived from an Expi293F™ GnTI KO Cell.

Embodiment 69: The cell of any one of Embodiments 61 to 68, wherein the cell further comprises a payload nucleic acid configured to encode a recombinant glycoprotein, and the expression of the payload nucleic acid is controlled by a constitutive or an activable promoter.

Embodiment 70: The cell of Embodiment 69, wherein the recombinant glycoprotein is an antibody or an antigen-binding fragment thereof.

Embodiment 71: The cell of Embodiment 70, wherein the antibody is a therapeutic antibody.

Embodiment 72: A method for glycoengineering a recombinant glycoprotein, comprising: delivering an expression vector into a cell according to any one of Embodiments 61 to 68, wherein the expression vector comprises a payload nucleic acid configured to encode a recombinant glycoprotein; and expressing the payload nucleic acid in the cell, thereby obtaining a plurality of recombinant glycoproteins, where at least one recombinant glycoprotein of the plurality is conjugated with a GlcNAc glycan.

Embodiment 73: The method of Embodiment 72, wherein the GlcNAc glycan is a mono-GlcNAc glycan or a GlcNAc-Fuc glycan.

Embodiment 74: The method of Embodiment 72 or Embodiment 73, wherein the expression of the payload nucleic acid is constitutive or controllable.

Embodiment 75: The method of any one of Embodiments 72 to 74, wherein at least 50% of the plurality of recombinant glycoproteins is conjugated with the GlcNAc glycan.

Embodiment 76: The method of any one of Embodiments 72 to 75, wherein the plurality of recombinant glycoproteins is conjugated with the GlcNAc glycan at homogeneity of at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Embodiment 77: The method of any one of Embodiments 72 to 76, further comprising harvesting the plurality of recombinant glycoproteins within 200 hours from the expression of the payload nucleic acid in the cell.

Embodiment 78: The method of Embodiment 77, wherein the plurality of recombinant glycoproteins was harvested within 100 hours from the expression of the payload nucleic acid in the cell.

Embodiment 79: The method of any one of Embodiments 72 to 78, further comprising: collecting the plurality of recombinant glycoproteins; and incubating the recombinant glycoproteins with an endoglycosidase and a donor glycan.

Embodiment 80: The method of Embodiment 79, wherein the donor glycan comprises an oxazoline moiety.

Embodiment 81: The method of Embodiment 79 or Embodiment 80, wherein the donor glycan is a sialyl complex type (SCT) glycan.

Embodiment 82: The method of any one of Embodiments 79 to 81, wherein the endoglycosidase is an endoglycosidase H (Endo H) or an endoglycosidase S2 (Endo S2).

Embodiment 83: The method of any one of Embodiments 72 to 82, wherein the recombinant glycoprotein is an antibody or an antigen-binding fragment thereof.

Embodiment 84: The method of Embodiment 83, wherein the glycan is on a constant region of the antibody.

Embodiment 85: The method of Embodiment 84, wherein the glycan is on a heavy chain of the antibody.

Embodiment 86: The method of any one of Embodiments 83 to 85, wherein the glycan is on a glycosylation site of the antibody.

Embodiment 87: The method of Embodiment 86, wherein the glycan is located at an N46, N402, and/or N297 site of the antibody.

Embodiment 88: The method of any one of Embodiments 83 to 87, wherein the antibody is a therapeutic antibody.

Embodiment 89: The method of Embodiment 88, wherein the therapeutic antibody is Adalimumab (Humira®), Adalimumab-atto (Amjevita®), Rituximab (Rituxan®), Rituximab-atto (Truxima®), Cetuximab (Erbitux®), Bevacizumab (Avastin®), Infliximab (Remicade®), Trastuzumab (Herceptin®), Pembrolizumab (Keytruda®), Etanercept (Enbrel®), Ipilimumab (Yervoy®), Ofatumumab (Arzerra), Golimumab (Simponi®), Atezolizumab (Tecentriq®), Ocrelizumab (OCREVUS®), Durvalumab (Durvalumab®), Avelumab (Bavencio®), Nivolumab (Opdivo®), Pertuzumab (Perjeta®), Obinutuzumab (Gazyva®), Gazyvaro Infliximab (Remicade®), or Trastuzumab emtansine (Kadcyla®).

Embodiment 90: A plurality of enriched recombinant glycoproteins, wherein at least 50% of the plurality of recombinant glycoproteins is configured with a GlcNAc glycan.

Embodiment 91: The plurality of enriched recombinant glycoproteins of Embodiment 90, wherein at least 70% of the plurality of recombinant glycoproteins is configured with the GlcNAc glycan.

Embodiment 92: The plurality of enriched recombinant glycoproteins of Embodiment 90 or Embodiment 91, wherein plurality of the recombinant glycoproteins is conjugated with the GlcNAc glycan at homogeneity of at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%

Embodiment 93: The plurality of enriched recombinant glycoproteins of Embodiment 90 or Embodiment 91, wherein plurality of the recombinant glycoproteins is conjugated with the GlcNAc glycan at homogeneity of 1% to 100%, 1% to 90%, 1% to 80%, 1% to 70%, 1% to 60%, 1% to 50%, 1% to 40%, 1% to 30%, 1% to 20%, 1% to 10%, 5% to 100%, 5% to 95%, 5% to 85%, 5% to 75%, 5% to 65%, 5% to 55%, 5% to 45%, 5% to 35%, 5% to 25%, 5% to 10%, 10% to 100%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 100%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 100%, 40% to 99%, 40% to 95%, 40% to 90%, 40% to 85%, 40% to 80%, 40% to 75%, 40% to 70%, 40% to 65%, 40% to 60%, 40% to 55%, 40% to 50%, 60% to 100%, 60% to 99%, 60% to 95%, 60% to 90%, 60% to 85%, 60% to 80%, 60% to 75%, 50% to 100%, 50% to 99%, 50% to 95%, 50% to 90%, 50% to 85%, 50% to 80%, 50% to 75%, 50% to 70%, 50% to 65%, 50% to 60%, 70% to 100%, 70% to 99%, 70% to 95%, 70% to 90%, 70% to 85%, 70% to 80%, 70% to 75%, 80% to 100%, 80% to 99%, 80% to 95%, 80% to 90%, 80% to 85%, 90% to 100%, 90% to 99%, or 90% to 95%.

Embodiment 94: The plurality of enriched recombinant glycoproteins of any one of Embodiments 90 to 93, wherein the GlcNAc glycan is a mono-GlcNAc glycan or a GlcNAc-Fuc glycan.

Embodiment 95: The plurality of enriched recombinant glycoproteins of any one of Embodiments 90 to 94, wherein the recombinant glycoprotein is an antibody or an antigen-binding fragment thereof.

Embodiment 96: The plurality of enriched recombinant glycoproteins of Embodiment 95, wherein the glycan is on a constant region of the antibody.

Embodiment 97: The plurality of enriched recombinant glycoproteins of Embodiment 96, wherein the glycan is on a heavy chain of the antibody.

Embodiment 98: The plurality of enriched recombinant glycoproteins of any one of Embodiments 95 to 97, wherein the glycan is on a glycosylation site of the antibody.

Embodiment 99: The plurality of enriched recombinant glycoproteins of Embodiment 98, wherein the glycan is on an N46, N402, and/or N297 site of the antibody.

Embodiment 100: The plurality of enriched recombinant glycoproteins of any one of Embodiments 95 to 99, wherein the antibody is a therapeutic antibody.

Embodiment 101: The plurality of enriched recombinant glycoproteins of any one of Embodiments 90 to 100, being obtained by using the method for glycoengineering a recombinant glycoprotein of any one of Embodiments 72 to 89.

SEQUENCES

| | |
|---|---|
| SEQ ID NO: 01 | MIHTNLKKKFSCCVLVFLLFAVICVWKEKKKGSYYDSFKLQTKEFQVLKSLGKLAM |
| ST6GAL1, aa | GSDSQSVSSSSTQDPHRGRQTLGSLRGLAKAKPEASFQVWNKDSSSKNLIPRLQKIW |
| | KNYLSMNKYKVSYKGPGPGIKFSAEALRCHLRDHVNVSMVEVTDFPFNTSEWEGYL |
| | PKESIRTKAGPWGRCAVVSSAGSLKSSQLGREIDDHDAVLRFNGAPTANFQQDVGTK |
| | TTIRLMNSQLVTTEKRFLKDSLYNEGILIVWDPSVYHSDIPKWYQNPDYNFFNNYKT |
| | YRKLHPNQPFYILKPQMPWELWDILQEISPEEIQPNPPSSGMLGIIIMMTLCDQVDIYE |
| | FLPSKRKTDVCYYYQKFFDSACTMGAYHPLLYEKNLVKHLNQGTDEDIYLLGKATLP |
| | GFRTIHC |
| SEQ ID NO: 02 | NVVAPSLEVYVDHASLPTLQQLMDIIKSEEENPTAQRYIAWGRIVPTDEQMKELNITS |
| PspST only, aa | FALINNHTPADLVQEIVKQAQTKHRLNVKLSSNTAHSFDNLVPILKELNSFNNVTVTNI |
| | DLYDDGSMEYVNLYNWRDTLNKTDNLKIGKDYLEDVINGINEDTSNTGTSSVYNWQ |
| | KLYPANYHFLRKDYLTLEPSLHELRDYIGDSLKQMQWDGFKKFNSKQQELFLSIVNF |
| | DKQKLQNEYNSSNLPNFVFTGTTVWGGNHEREYYAKQQINVINNAINESSPHYLGNS |
| | YDLFFKGHPGGGIINTLIMQNYPSMVDIPSKISFEVLMMTDMLPDAVAGIASSLYFTIP |
| | AEKIKFIVFTSTETITDRETALRSPLVQVMIKLGIVKEENVLFWADLPNCETGVC |
| SEQ ID NO: 03 | MIHTNLKKKFSCCVLVFLLFAVICVWKEKKKGSYYDSFKLQTKEFQVLKSLGKLAM |
| Modified PspST | GSDSQSVSSSSTQDPHRGRQTLGSLRGLAKAKPGGGGSGGGGSGGGGSNVVAPSLE |
| (ST6GAL1 | VYVDHASLPTLQQLMDIIKSEEENPTAQRYIAWGRIVPTDEQMKELNITSFALINNHTP |
| signal peptide), | ADLVQEIVKQAQTKHRLNVKLSSNTAHSFDNLVPILKELNSFNNVTVTNIDLYDDGS |
| aa | MEYVNLYNWRDTLNKTDNLKIGKDYLEDVINGINEDTSNTGTSSVYNWQKLYPANY |
| | HFLRKDYLTLEPSLHELRDYIGDSLKQMQWDGFKKFNSKQQELFLSIVNFDKQKLQN |
| | EYNSSNLPNFVFTGTTVWGGNHEREYYAKQQINVINNAINESSPHYLGNSYDLFFKG |
| | HPGGGIINTLIMQNYPSMVDIPSKISFEVLMMTDMLPDAVAGIASSLYFTIPAEKIKFIV |
| | FTSTETITDRETALRSPLVQVMIKLGIVKEENVLFWADLPNCETGVCGSGATNFSLLK |
| | QAGDVEENPGPMRLREPLLSGSAAMPGASLQRACRLLVAVCALHLGVTLVYYLAGR |
| | DLSRLPQLVGVSTPLQGGSNSAAAIGQSSGELRTGGARPPPPLGASSQPRPGGDSSPV |
| | VDSGPGPASNLTSVPVPHTTALSLPACPEESPLLVGPMLIEFNMPVDLELVAKQNPNVK |
| | MGGRYAPRDCVSPHKVAIIIPFRNRQEHLKYWLYYLHPVLQRQQLDYGIYVINQAGD |
| | TIFNRAKLLNVGFQEALKDYDYTCFVFSDVDLIPMNDHNAYRCFSQPRHISVAMDKF |
| | GFSLPYVQYFGGVSALSKQQFLTINGFPNNYWGWGGEDDDIFNRLVFRGMSISRPNA |
| | VVGRCRMIRHSRDKKNEPNPQRFDRIAHTKETMLSDGLNSLTYQVLDVQRYPLYTQI |
| | TVDIGTPS |
| SEQ ID NO: 04 | MRLREPLLSGSAAMPGASLQRACRLLVAVCALHLGVTLVYYLAGRDLSRLPQLVGVS |
| Modified PspST | TPLQGGSNSAAAIGQSSGELRTGGARPPPPLGASSQPRPGGGGGGSGGGGSGGGGSN |
| (B4GALT1 | VVAPSLEVYVDHASLPTLQQLMDIIKSEEENPTAQRYIAWGRIVPTDEQMKELNITSFA |
| signal peptide), | LINNHTPADLVQEIVKQAQTKHRLNVKLSSNTAHSFDNLVPILKELNSFNNVTVTNID |
| aa | LYDDGSMEYVNLYNWRDTLNKTDNLKIGKDYLEDVINGINEDTSNTGTSSVYNWQK |
| | LYPANYHFLRKDYLTLEPSLHELRDYIGDSLKQMQWDGFKKFNSKQQELFLSIVNFD |
| | KQKLQNEYNSSNLPNFVFTGTTVWGGNHEREYYAKQQINVINNAINESSPHYLGNSY |
| | DLFFKGHPGGGIINTLIMQNYPSMVDIPSKISFEVLMMTDMLPDAVAGIASSLYFTIPAE |
| | KIKFIVFTSTETITDRETALRSPLVQVMIKLGIVKEENVLFWADLPNCETGVCGSGATN |
| | FSLLKQAGDVEENPGPMRLREPLLSGSAAMPGASLQRACRLLVAVCALHLGVTLVYY |
| | LAGRDLSRLPQLVGVSTPLQGGSNSAAAIGQSSGELRTGGARPPPPLGASSQPRPGGD |
| | SSPVVDSGPGPASNLTSVPVPHTTALSLPACPEESPLLVGPMLIEFNMPVDLELVAKQN |
| | PNVKMGGRYAPRDCVSPHKVAIIIPFRNRQEHLKYWLYYLHPVLQRQQLDYGIYVIN |
| | QAGDTIFNRAKLLNVGFQEALKDYDYTCFVFSDVDLIPMNDHNAYRCFSQPRHISVA |
| | MDKFGFSLPYVQYFGGVSALSKQQFLTINGFPNNYWGWGGEDDDIFNRLVFRGMSIS |
| | RPNAVVGRCRMIRHSRDKKNEPNPQRFDRIAHTKETMLSDGLNSLTYQVLDVQRYPL |
| | YTQITVDIGTPS |
| SEQ ID NO: 05 | MRLREPLLSGSAAMPGASLQRACRLLVAVCALHLGVTLVYYLAGRDLSRLPQLVGVS |
| B4GALT1, aa | TPLQGGSNSAAAIGQSSGELRTGGARPPPPLGASSQPRPGGDSSPVVDSGPGPASNLTS |
| | VPVPHTTALSLPACPEESPLLVGPMLIEFNMPVDLELVAKQNPNVKMGGRYAPRDCVS |
| | PHKVAIIIPFRNRQEHLKYWLYYLHPVLQRQQLDYGIYVINQAGDTIFNRAKLLNVGF |
| | QEALKDYDYTCFVFSDVDLIPMNDHNAYRCFSQPRHISVAMDKFGFSLPYVQYFGGV |
| | SALSKQQFLTINGFPNNYWGWGGEDDDIFNRLVFRGMSISRPNAVVGRCRMIRHSRD |
| | KKNEPNPQRFDRIAHTKETMLSDGLNSLTYQVLDVQRYPLYTQITVDIGTPS |
| SEQ ID NO: 06 | GSGATNFSLLKQAGDVEENPGP |
| Ribosomal | |
| Shifting peptide- | |
| P2A, aa | |
| SEQ ID NO: 07 | GSGEGRGSLLTCGDVEENPGP |
| Ribosomal | |
| Shifting peptide- | |
| T2A, aa | |
| SEQ ID NO: 08 | GSGQCTNYALLKLAGDVESNPGP |
| Ribosomal | |
| Shifting peptide- | |
| E2A, aa | |
| SEQ ID NO: 09 | GSGVKQTLNFDLLKLAGDVESNPGP |
| Ribosomal | |
| Shifting peptide- | |
| F2A, aa | |
| SEQ ID NO: 10 | ATGATCCACACCAATCTGAAGAAGAAGTTTAGCTGTTGCGTGCTGGTGTTCCTGCT |
| ST6GAL1, nt | GTTCGCTGTGATCTGCGTGTGGAAGGAGAAGAAGAAGGGCAGCTATTATGACTCC |
| | TTTAAGCTGCAGACCAAGGAGTTCCAGGTGCTGAAGTCCCTGGGCAAGCTGGCC |
| | ATGGGCAGCGACTCCCAGTCCGTGAGCTCCTCCAGCACCCAGGACCCCCACCGG |
| | GGAAGGCAGACCCTGGGATCCCTGAGGGGCCTGGCTAAGGCCAAGCCTGAGGCT |
| | TCCTTCCAGGTGTGGAATAAGGATAGCTCCAGCAAGAACCTGATCCCTAGGCTGC |
| | AGAAGATCTGGAAGAACTATCTGAGCATGAACAAGTATAAGGTGAGCTATAAGGG |
| | CCCTGGCCCTGGCATCAAGTTCTCCGCCGAGGCCCTGAGGTGCCACCTGAGAGAT |
| | CACGTGAATGTGAGCATGGTGGAGGTGACCGACTTCCCTTTTAACACCTCCGAGT |
| | GGGAGGGCTACCTGCCCAAGGAGAGCATCCGGACCAAGGCCGGCCCTTGGGGCA |
| | GATGTGCCGTGGTGTCCAGCGCCGGCTCCCTGAAGAGCTCCCAGCTGGGCCGGG |
| | AGATCGATGACCACGATGCTGTGCTGAGGTTCAATGGCGCTCCTACCGCTAACTTT |
| | CAGCAGGATGTGGGCACCAAGACCACCATCCGGCTGATGAATAGCCAGCTGGTGA |
| | CCACCGAGAAGCGGTTTCTGAAGGACTCCCTGTACAACGAGGGCATCCTGATCGT |
| | GTGGGACCCCTCCGTGTACCACAGCGACATCCCCAAGTGGTATCAGAACCCCGAC |
| | TACAACTTCTTCAATAATTACAAGACCTACCGGAAGCTGCACCCCAACCAGCCCTT |
| | TTATATCCTGAAGCCCCAGATGCCTTGGGAGCTGTGGGACATCCTGCAGGAGATCT |
| | CCCCCGAGGAGATCCAGCCTAACCCCCCTAGCAGCGGCATGCTGGGCATCATCATC |
| | ATGATGACCCTGTGCGATCAGGTGGATATCTACGAGTTCCTGCCCAGCAAGCGGA |
| | AGACCGACGTGTGTTATTATTATCAGAAGTTCTTCGATTCCGCCTGCACCATGGGC |
| | GCCTATCACCCTCTGCTGTACGAGAAGAATCTGGTGAAGCACCTGAACCAGGGCA |
| | CCGACGAGGACATCTATCTGCTGGGCAAGGCCACCCTGCCCGGCTTTAGGACCAT |
| | CCACTGCTGA |
| SEQ ID NO: 11 | ATGAAAAACTTTTTATTATTAACTTTAATATTACTTACTGCTTGTAATAATTCAGAAG |
| PspST only, nt | AAAATACACAATCTATTATTAAAAATGATATTAATAAAACTATTATTGATGAGGAGTA |
| | TGTTAATTTAGAGCCAATTAATCAATCAAACATCTCTTTTACAAAACACTCTTGGGT |
| | ACAAACTTGTGGTACGCAACAACTATTAACAGAACAAAATAAAGAGTCAATATCA |
| | TTATCTGTAGTGGCGCCACGATTAGATGACGATGAAAAGTACTGCTTTGATTTTAAT |
| | GGTGTTAGTAATAAAGGTGAAAAATATATAACAAAAGTAACATTAAACGTAGTGGC |
| | TCCATCTTTAGAGGTTTATGTTGATCATGCATCTCTTCCAACTCTTCAGCAGCTAAT |
| | GGATATTATTAAATCGGAAGAAGAAAATCCTACAGCACAAAGATATATAGCTTGGG |
| | GGAGAATAGTTCCGACTGATGAGCAAATGAAAGAGTTAAATATTACATCGTTTGCA |
| | TTGATAAATAACCATACACCAGCTGACTTAGTACAAGAAATTGTTAAGCAAGCACA |
| | AACAAAGCATAGATTGAATGTTAAACTTAGCTCTAACACTGCTCATTCATTTGATAA |
| | TTTAGTGCCAATACTAAAAGAATTAAATTCGTTTAATAACGTTACGGTAACAAATAT |
| | AGATTTATATGATGATGGTTCAGCAGAATATGTAAATTTATATAACTGGAGAGATAC |
| | ATTAAATAAAACAGATAATTTAAAAATTGGTAAAGATTATCTTGAGGATGTCATTAA |
| | TGGTATCAATGAAGACACTTCAAATACAGGAACATCATCTGTTTATAACTGGCAAA |
| | AACTATATCCAGCTAACTACCATTTTTTAAGAAAAGATTATTTAACTTTAGAACCAT |
| | CATTACATGAGTTACGAGACTATATTGGTGATAGTTTAAAGCAAATGCAATGGGATG |
| | GTTTCAAAAAATTCAATAGCAAACAACAAGAATTATTCTTATCGATTGTTAATTTTG |
| | ACAAACAAAAATTACAAAATGAATATAATTCATCTAATTTACCAAACTTTGTGTTTA |
| | CAGGTACGACTGTATGGGCTGGTAACCATGAAAGAGAGTATTATGCGAAACAACA |
| | AATTAATGTCATTAATAATGCAATTAATGAATCGAGCCCACATTATTTAGGCAATAGT |
| | TATGATTTGTTCTTCAAAGGTCACCCTGGTGGCGGTATCATTAATACATTAATAATG |
| | CAAAACTATCCTTCAATGGTTGATATTCCATCAAAAATATCATTTGAAGTTTTGATG |
| | ATGACAGATATGCTTCCTGATGCAGTTGCTGGTATAGCGAGCTCTTTATATTTCACG |
| | ATACCAGCTGAAAAAATTAAATTTATAGTTTTTACATCGACAGAAACTATAACTGAT |
| | CGTGAAACTGCTTTGAGAAGTCCTTTAGTTCAAGTAATGATAAAACTAGGTATTGT |
| | AAAAGAAGAGAATGTACTTTTTTGGGCTGATCTGCCAAATTGTGAAACAGGTGTT |
| | TGTATTGCAGTCTAG |
| SEQ ID NO: 12 | ATGATCCACACCAACCTGAAGAAGAAGTTCTCCTGCTGTGTGCTGGTGTTTCTGCT |
| Modified PspST | GTTCGCTGTGATCTGCGTGTGGAAGGAGAAGAAGAAGGGCAGCTATTATGACAGC |
| (ST6GAL1 | TTTAAGCTGCAGACCAAGGAGTTTCAGGTGCTGAAGTCCCTGGGCAAGCTGGCCA |
| signal peptide), | TGGGCTCCGACTCCCAGTCCGTGTCCAGCTCCAGCACCCAGGATCCTCACCGGGG |
| nt | CAGGCAGACCCTGGGCTCTCTGAGAGGCCTGGCTAAGGCTAAGCCCGGCGGCGG |
| | AGGCAGCGGAGGAGGAGGATCTGGCGGCGGAGGATCCAATGTGGTGGCCCCTTC |
| | CCTGGAGGTGTATGTGGACCACGCCTCCCTGCCCACCCTGCAGCAGCTGATGGAT |
| | ATCATCAAGAGCGAGGAGGAGAATCCTACCGCCCAGCGGTATATCGCCTGGGGCC |
| | GGATCGTGCCTACCGACGAGCAGATGAAGGAGCTGAACATCACCTCCTTCGCTCT |
| | GATCAACAATCACACCCCCGCCGACCTGGTGCAGGAGATCGTGAAGCAGGCCCA |
| | GACCAAGCACCGGCTGAACGTGAAGCTGTCCTCCAACACCGCCCACTCCTTTGAC |
| | AACCTGGTGCCTATCCTGAAGGAGCTGAATTCCTTTAATAACGTGACCGTGACCAA |
| | CATCGACCTGTACGACGACGGCTCCATGGAGTATGTGAACCTGTATAACTGGAGG |
| | GACACCCTGAATAAGACCGACAATCTGAAGATCGGCAAGGACTATCTGGAGGACG |
| | TGATCAATGGCATCAATGAGGACACCTCCAACACCGGCACCAGCAGCGTGTATAA |
| | CTGGCAGAAGCTGTACCCTGCTAATTACCACTTTCTGAGGAAGGATTATCTGACCC |
| | TGGAGCCTTCCCTGCACGAGCTGAGGGACTACATCGGCGATAGCCTGAAGCAGAT |
| | GCAGTGGGATGGCTTTAAGAAGTTCAACAGCAAGCAGCAGGAGCTGTTTCTGAG |
| | CATCGTGAATTTTGACAAGCAGAAGCTGCAGAACGAGTATAACTCCTCCAATCTG |
| | CCTAACTTTGTGTTCACCGGCACCACCGTGTGGGGCGGCAATCACGAGCGGGAGT |
| | ACTATGCCAAGCAGCAGATCAACGTGATCAATAATGCCATCAATGAGAGCAGCCC |
| | CCACTACCTGGGCAATAGCTACGATCTGTTCTTCAAGGGCCACCCTGGCGGCGGC |
| | ATCATCAACACCCTGATCATGCAGAACTATCCTTCCATGGTGGATATCCCTAGCAAG |
| | ATCTCCTTCGAGGTGCTGATGATGACCGACATGCTGCCCGACGCCGTGGCTGGCAT |
| | CGCCTCTAGCCTGTATTTTACCATCCCCGCTGAGAAGATCAAGTTCATCGTGTTTAC |
| | CAGCACCGAGACCATCACCGACCGGGAGACCGCCCTGAGGTCCCCACTGGTGCA |
| | GGTGATGATCAAGCTGGGCATCGTGAAGGAGGAGAACGTGCTGTTTTGGGCCGAC |
| | CTGCCCAACTGTGAGACCGGCGTGTGTGGCTCCGGCGCTACCAACTTCTCCCTGC |
| | TGAAGCAGGCTGGCGATGTGGAGGAGAATCCCGGCCCTATGCGGCTGCGGGAGC |
| | CCCTGTTGTCCGGCTCTGCCGCTATGCCCGGCGCTTCCCTGCAGAGAGCCTGTCGG |
| | CTGCTGGTGGCTGTGTGCGCTCTGCACCTGGGCGTGACCCTGGTGTACTATCTGGC |
| | TGGCCGGGATCTGAGCCGGCTGCCTCAGCTGGTGGGCGTGAGCACCCCCCTGCAG |
| | GGAGGATCCAACTCCGCCGCTGCCATCGGCCAGTCCTCCGGAGAGCTGCGGACCG |
| | GAGGCGCTAGGCCTCCACCACCACTGGGCGCTTCTTCCCAGCCTCGGCCCGGAGG |
| | AGATAGCAGCCCCGTGGTGGACTCCGGCCCTGGACCTGCTTCCAACCTGACCAGC |
| | GTGCCCGTGCCTCACACCACCGCTCTGTCCCTGCCCGCCTGTCCCGAGGAGTCCC |
| | CTCTGCTGGTGGGCCCTATGCTGATCGAGTTTAATATGCCTGTGGACCTGGAGCTG |
| | GTGGCTAAGCAGAATCCTAACGTGAAGATGGGCGGCAGGTACGCTCCCAGGGACT |
| | GCGTGAGCCCTCACAAGGTGGCCATCATCATCCCTTTCCGGAACCGGCAGGAGCA |
| | CCTGAAGTACTGGCTGTACTACCTGCACCCTGTGCTGCAGAGGCAGCAGCTGGAC |
| | TATGGCATCTACGTGATCAACCAGGCCGGCGACACCATCTTTAACCGGGCCAAGCT |
| | GCTGAATGTGGGCTTTCAGGAGGCCCTGAAGGATTATGACTACACCTGCTTTGTGT |
| | TTAGCGATGTGGATCTGATCCCTATGAACGATCACAACGCCTACCGGTGCTTCAGC |
| | CAGCCTCGGCACATCAGCGTGGCCATGGACAAGTTCGGCTTCTCCCTGCCTTACGT |
| | GCAGTACTTCGGCGGCGTGTCCGCCCTGTCCAAGCAGCAGTTCCTGACCATCAAC |
| | GGCTTTCCTAATAACTACTGGGGCTGGGGCGGCGAGGATGATGACATCTTTAATCG |
| | GCTGGTGTTTAGGGGCATGAGCATCAGCCGGCCTAACGCCGTGGTGGGCAGGTGC |
| | AGGATGATCAGGCACTCCCGGGACAAGAAGAACGAGCCTAATCCTCAGAGGTTTG |
| | ACAGGATCGCTCACACCAAGGAGACCATGCTGAGCGACGGCCTGAATAGCCTGAC |
| | CTACCAGGTGCTGGACGTGCAGAGGTACCCTCTGTATACCCAGATCACCGTGGATA |
| | TCGGCACCCCTAGCTGA |
| SEQ ID NO: 13 | ATGAGGCTGCGGGAGCCATTGCTGAGCGGCTCCGCTGCCATGCCTGGCGCTTCTC |
| Modified PspST | TGCAGAGGGCTTGTAGGCTGCTGGTGGCCGTGTGCGCCCTGCACCTGGGAGTGAC |
| (B4GALT1 | CCTGGTGTACTACCTGGCTGGCCGGGACCTGTCCCGGCTGCCTCAGTTGGTGGGC |
| signal peptide), | GTGTCCACCCCTCTGCAGGGCGGCGGAGGCAGCGGAGGAGGAGGATCTGGCGGC |
| nt | GGAGGATCCAATGTGGTGGCCCCTTCCCTGGAGGTGTATGTGGACCACGCCTCCC |
| | TGCCCACCCTGCAGCAGCTGATGGATATCATCAAGAGCGAGGAGGAGAATCCTAC |
| | CGCCCAGCGGTATATCGCCTGGGGCCGGATCGTGCCTACCGACGAGCAGATGAAG |
| | GAGCTGAACATCACCTCCTTCGCTCTGATCAACAATCACACCCCCGCCGACCTGG |
| | TGCAGGAGATCGTGAAGCAGGCCCAGACCAAGCACCGGCTGAACGTGAAGCTGT |
| | CCTCCAACACCGCCCACTCCTTTGACAACCTGGTGCCTATCCTGAAGGAGCTGAA |
| | TTCCTTTAATAACGTGACCGTGACCAACATCGACCTGTACGACGACGGCTCCATGG |
| | AGTATGTGAACCTGTATAACTGGAGGGACACCCTGAATAAGACCGACAATCTGAA |
| | GATCGGCAAGGACTATCTGGAGGACGTGATCAATGGCATCAATGAGGACACCTCC |
| | AACACCGGCACCAGCAGCGTGTATAACTGGCAGAAGCTGTACCCTGCTAATTACC |
| | ACTTTCTGAGGAAGGATTATCTGACCCTGGAGCCTTCCCTGCACGAGCTGAGGGA |
| | CTACATCGGCGATAGCCTGAAGCAGATGCAGTGGGATGGCTTTAAGAAGTTCAAC |
| | AGCAAGCAGCAGGAGCTGTTTCTGAGCATCGTGAATTTTGACAAGCAGAAGCTGC |
| | AGAACGAGTATAACTCCTCCAATCTGCCTAACTTTGTGTTCACCGGCACCACCGTG |
| | TGGGGCGGCAATCACGAGCGGGAGTACTATGCCAAGCAGCAGATCAACGTGATCA |
| | ATAATGCCATCAATGAGAGCAGCCCCCACTACCTGGGCAATAGCTACGATCTGTTC |
| | TTCAAGGGCCACCCTGGCGGCGGCATCATCAACACCCTGATCATGCAGAACTATCC |
| | TTCCATGGTGGATATCCCTAGCAAGATCTCCTTCGAGGTGCTGATGATGACCGACA |
| | TGCTGCCCGACGCCGTGGCTGGCATCGCCTCTAGCCTGTATTTTACCATCCCCGCT |
| | GAGAAGATCAAGTTCATCGTGTTTACCAGCACCGAGACCATCACCGACCGGGAGA |
| | CCGCCCTGAGGTCCCCACTGGTGCAGGTGATGATCAAGCTGGGCATCGTGAAGGA |
| | GGAGAACGTGCTGTTTTGGGCCGACCTGCCCAACTGTGAGACCGGCGTGTGT |
| SEQ ID NO: 14 | ATGAGGCTGCGGGAGCCATTGCTGAGCGGCTCCGCTGCCATGCCTGGCGCTTCTC |
| B4GALT1, nt | TGCAGAGGGCTTGTAGGCTGCTGGTGGCCGTGTGCGCCCTGCACCTGGGAGTGAC |
| | CCTGGTGTACTACCTGGCTGGCCGGGACCTGTCCCGGCTGCCTCAGTTGGTGGGC |
| | GTGTCCACCCCTCTGCAGGGCGGAAGCAATAGCGCCGCTGCCATCGGCCAGTCCT |
| | CCGGAGAGCTGCGGACCGGAGGCGCTAGGCCACCTCCACCTCTGGGCGCTTCCTC |
| | CCAGCCTCGGCCTGGAGGAGATAGCAGCCCCGTGGTGGATAGCGGCCCTGGCCCA |
| | GCTTCTAACCTGACCAGCGTGCCTGTGCCCCACACCACCGCCCTGAGCCTGCCTG |
| | CTTGCCCCGAGGAGTCCCCCCTGCTGGTGGGACCAATGCTGATCGAGTTCAATATG |
| | CCTGTGGATCTGGAGCTGGTGGCCAAGCAGAATCCCAATGTGAAGATGGGCGGCC |
| | GGTACGCTCCCCGGGATTGTGTGTCCCCTCACAAGGTGGCTATCATCATCCCTTTC |
| | CGGAACCGGCAGGAGCACCTGAAGTACTGGCTGTATTACCTGCACCCCGTGCTGC |
| | AGAGGCAGCAGCTGGACTATGGCATCTACGTGATCAATCAGGCTGGCGACACCAT |
| | CTTCAATCGGGCTAAGCTGCTGAACGTGGGCTTTCAGGAGGCTCTGAAGGACTAC |
| | GACTACACCTGCTTTGTGTTCAGCGATGTGGACCTGATCCCCATGAACGACCACA |
| | ACGCTTATAGGTGCTTCTCCCAGCCTAGGCACATCTCCGTGGCTATGGACAAGTTT |
| | GGCTTCTCCCTGCCCTACGTGCAGTATTTTGGCGGCGTGTCCGCCCTGAGCAAGCA |
| | GCAGTTTCTGACCATCAATGGCTTTCCTAACAATTACTGGGGCTGGGGCGGCGAG |
| | GATGATGACATCTTCAACCGGCTGGTGTTCAGGGGCATGTCCATCAGCAGGCCTAA |
| | CGCTGTGGTGGGCCGGTGTAGGATGATCAGGCACTCCCGGGACAAGAAGAATGA |
| | GCCCAATCCTCAGCGGTTTGACCGGATCGCCCACACCAAGGAGACCATGCTGTCC |
| | GACGGCCTGAATTCCCTGACCTACCAGGTGCTGGACGTGCAGAGGTATCCTCTGT |
| | ACACCCAGATCACCGTGGATATCGGCACCCCTTCCTGA |
| SEQ ID NO: 15 | MFTPVRRRVRTAALALSAAAALVLGSTAASGASATPSPAPAPAPAPVKQGPTSVAYVE |
| Endo H, aa | VNNNSMLNVGKYTLADGGGNAFDVAVIFAANINYDTGTKTAYLHFNENVQRVLDNA |
| | VTQIRPLQQQGIKVLLSVLGNHQGAGFANFPSQQAASAFAKQLSDAVAKYGLDGVDF |
| | DDEYAEYGNNGTAQPNDSSFVHLVTALRANMPDKIISLYNIGPAASRLSYGGVDVSD |
| | KFDYAWNPYYGTWQVPGIALPKAQLSPAAVEIGRTSRSTVADLARRTVDEGYGVYLT |
| | YNLDGGDRTADVSAFTRELYGSEAVRTP |
| SEQ ID NO: 16 | MDKHLLVKRTLGCVCAATLMGAALATHHDSLNTVKAEEKTVQTGKTDQQVGAKLV |
| Endo S2, aa | QEIREGKRGPLYAGYFRTWHDRASTGIDGKQQHPENTMAEVPKEVDILFVFHDHTAS |
| | DSPFWSELKDSYVHKLHQQGTALVQTIGVNELNGRTGLSKDYPDTPEGNKALAAAIV |
| | KAFVTDRGVDGLDIDIEHEFTNKRTPEEDARALNVFKEIAQLIGKNGSDKSKLLIMDT |
| | TLSVENNPIFKGIAEDLDYLLRQYYGSQGGEAEVDTINSDWNQYQNYIDASQFMIGF |
| | SFFEESASKGNLWFDVNEYDPNNPEKGKDIEGTRAKKYAEWQPSTGGLKAGIFSYAI |
| | DRDGVAHVPSTYKNRTSTNLQRHEVDNISHTDYTVSRKLKTLMTEDKRYDVIDQKDI |
| | PDPALREQIIQQVGQYKGDLERYNKTLVLTGDKIQNLKGLEKLSKLQKLELRQLSNV |
| | KEITPELLPESMKKDAELVMVGMTGLEKLNLSGLNRQTLDGIDVNSITHLTSFDISHN |
| | SLDLSEKSEDRKLLMTLMEQVSNHQKITVKNTAFENQKPKGYYPQTYDTKEGHYDV |
| | DNAEHDILTDFVFGTVTKRNTFIGDEEAFAIYKEGAVDGRQYVSKDYTYEAFRKDYK |
| | GYKVHLTASNLGETVTSKVTATTDETYLVDVSDGEKVVHHMKLNIGSGAIMMENLA |
| | KGAKVIGTSGDFEQAKKIFDGEKSDRFFTWGQTNWIAFDLGEINLAKEWRLFNAETN |
| | TEIKTDSSLNVAKGRLQILKDTTIDLEKMDIKNRKEYLSNDENWTDVAQMDDAKAIF |
| | NSKLSNVLSRYWRFCVDGGASSYYPQYTELQILGQRLSNDVANTLKD |
| SEQ ID NO: 17 | GACTCGAAGCACTCGCTCGGCGCTTCCGACTGGCGGGGCGCCGACCATCCCGTTG |
| Endo H, nt | GGAGACCATCAGCCATGTTCACTCCGGTTCGCAGAAGGGTGCGGACGGCTGCGCT |
| | CGCGCTCTCGGCCGCCGCGGCCCTCGTCCTCGGTTCCACCGCCGCGAGCGGCGCG |
| | TCAGCGACCCCCTCACCCGCTCCGGCCCCGGCCCCGGCCCCGGTGAAGCAGGGG |
| | CCGACCTCGGTGGCCTACGTCGAGGTGAACAACAACAGCATGCTCAACGTCGGC |
| | AAGTACACCCTGGCGGACGGAGGCGGCAACGCCTTCGACGTAGCCGTGATCTTCG |
| | CGGCGAACATCAACTACGACACCGGCACGAAGACGGCCTACCTGCACTTCAACG |
| | AGAACGTGCAGCGCGTCCTTGACAACGCTGTCACGCAGATACGGCCGTTGCAGCA |
| | ACAGGGCATCAAGGTCCTCCTCTCGGTGCTCGGCAACCACCAGGGCGCCGGGTTC |
| | GCGAACTTCCCCTCACAGCAGGCGGCTTCGGCGTTCGCGAAGCAGCTCTCGGAC |
| | GCCGTGGCGAAGTACGGCCTCGACGGCGTCGACTTCGACGACGAATACGCCGAG |
| | TACGGCAACAACGGCACCGCGCAGCCCAACGACAGTTCGTTCGTGCACCTGGTG |
| | ACGGCACTGCGCGCGAACATGCCCGACAAGATCATCAGCCTCTACAACATCGGCC |
| | CGGCCGCGTCCCGCCTGTCGTACGGCGGTGTCGACGTCTCCGACAAGTTCGACTA |
| | CGCCTGGAATCCCTACTACGGCACCTGGCAGGTCCCCGGCATCGCACTGCCCAAG |
| | GCGCAGCTGTCGCCGGCGGCCGTCGAGATCGGCCGGACCTCACGGAGCACCGTC |
| | GCCGACCTCGCCCGTCGCACCGTCGACGAGGGGTACGGCGTCTATCTGACGTACA |
| | ACCTCGACGGCGGCGATCGCACCGCCGACGTCTCCGCGTTCACCAGGGAGCTGTA |
| | CGGCAGCGAGGCGGTCCGGACGCCGTAGGGGCGTCGGGGCCTGCCGTCAGTCCA |
| | GTACGAAGGTGCCGCCGGCGGTGGTCGCCTGGCCGTGCCCGAAAGCGGCCGCCG |
| | GCGTCCAGGATCC |
| SEQ ID NO: 18 | GGCGAACTATAGGAATGCGCTTACATAGATGGTATATCAGATGGGAAGCCATGACT |
| Endo S2, nt | TAGTACCAAAAATAAGGAGTGTCCAAATGGATAAACATTTGTTGGTAAAAAGAAC |
| | ACTAGGGTGTGTTTGTGCTGCAACGTTGATGGGAGCTGCCTTAGCGACCCACCAT |
| | GATTCACTCAATACTGTAAAAGCGGAGGAGAAGACTGTTCAAACAGGAAAGACA |
| | GATCAGCAGGTTGGTGCTAAATTGGTACAGGAAATCCGTGAAGGAAAACGCGGA |
| | CCACTATATGCTGGTTATTTTAGGACATGGCATGATCGTGCTTCAACAGGAATAGAT |
| | GGTAAACAGCAACATCCAGAAAATACTATGGCTGAGGTCCCAAAAGAAGTTGATA |
| | TCTTATTTGTTTTTCATGACCATACAGCTTCAGATAGTCCATTTTGGTCTGAATTAAA |
| | GGACAGTTATGTCCATAAATTACATCAACAGGGAACGGCACTTGTTCAGACAATTG |
| | GTGTTAACGAATTAAATGGACGTACAGGTTTATCTAAAGATTATCCTGATACTCCTG |
| | AGGGGAACAAAGCTTTAGCAGCAGCCATTGTCAAGGCATTTGTAACTGATCGTGG |
| | TGTCGATGGACTAGATATTGATATTGAGCACGAATTTACGAACAAAAGAACACCTG |
| | AAGAAGATGCTCGTGCTCTAAATGTTTTTAAAGAGATTGCGCAGTTAATAGGTAAA |
| | AATGGTAGTGATAAATCTAAATTGCTCATCATGGACACTACCCTAAGTGTTGAAAA |
| | TAATCCAATATTTAAAGGGATAGCGGAAGATCTTGATTATCTTCTTAGACAATATTAT |
| | GGTTCACAAGGTGGAGAAGCTGAAGTGGATACTATAAACTCTGATTGGAACCAAT |
| | ATCAGAATTATATTGATGCTAGCCAGTTCATGATTGGATTCTCCTTTTTTGAAGAATC |
| | TGCGTCCAAAGGGAATTTATGGTTTGATGTTAACGAATACGACCCTAACAATCCTG |
| | AAAAAGGGAAAGATATTGAAGGAACACGTGCTAAAAAATATGCAGAGTGGCAAC |
| | CTAGTACAGGTGGTTTAAAAGCAGGTATATTCTCTTATGCTATTGATCGTGATGGAG |
| | TGGCTCATGTTCCTTCAACATATAAAAATAGGACTAGTACAAATTTACAACGGCAT |
| | GAAGTCGATAATATCTCACATACTGACTACACCGTATCTCGAAAATTAAAAACATTG |
| | ATGACCGAAGACAAACGCTATGATGTCATTGATCAAAAAGACATTCCTGACCCAG |
| | CATTAAGAGAACAAATCATTCAACAAGTTGGACAGTATAAAGGCGATTTGGAACG |
| | TTATAACAAGACATTGGTGCTTACAGGAGATAAGATTCAAAATCTTAAAGGACTAG |
| | AAAAATTAAGCAAGTTACAAAAATTAGAGTTGCGCCAGCTATCTAACGTTAAAGA |
| | AATTACTCCAGAACTTTTGCCGGAAAGCATGAAAAAAGATGCTGAGCTTGTTATG |
| | GTAGGCATGACTGGTTTAGAAAAACTAAACCTTAGTGGTCTAAATCGTCAAACTTT |
| | AGACGGTATAGACGTGAATAGTATTACGCATTTGACATCATTTGATATTTCACATAAT |
| | AGTTTGGACTTGTCGGAAAAGAGTGAAGACCGTAAACTATTAATGACTTTGATGG |
| | AGCAGGTTTCAAATCATCAAAAAATAACGGTGAAAAATACGGCTTTTGAAAATCA |
| | AAAACCGAAAGGTTATTATCCTCAGACGTATGATACCAAAGAAGGTCATTATGATG |
| | TTGATAATGCAGAACATGATATTTTAACTGATTTTGTTTTTGGAACTGTTACTAAAC |
| | GTAATACCTTTATTGGAGACGAAGAAGCATTTGCTATCTATAAAGAAGGAGCTGTC |
| | GATGGTCGACAATATGTGTCTAAAGACTATACTTATGAAGCTTTTCGTAAAGACTAT |
| | AAAGGTTACAAGGTTCATTTAACTGCTTCTAACCTAGGAGAAACAGTTACTTCTAA |
| | GGTAACTGCTACTACTGATGAAACTTACTTAGTAGATGTTTCTGATGGGGAAAAAG |
| | TTGTTCACCACATGAAACTCAATATAGGATCTGGTGCCATCATGATGGAAAATCTG |
| | GCAAAAGGGGCTAAAGTGATTGGTACATCTGGGGACTTTGAGCAAGCAAAGAAG |
| | ATTTTCGATGGTGAAAAGTCAGATAGATTCTTCACTTGGGGACAAACTAACTGGAT |
| | AGCTTTTGATCTAGGAGAAATTAATCTTGCGAAGGAATGGCGTTTATTTAATGCAG |
| | AGACAAATACTGAAATAAAGACAGATAGTAGCTTAAACGTGGCTAAAGGACGTCT |
| | TCAGATTTTAAAAGATACAACTATTGATTTAGAAAAAATGGACATAAAAAATCGTA |
| | AAGAGTATCTGTCGAATGATGAAAATTGGACTGATGTTGCTCAGATGGATGATGCA |
| | AAAGCGATATTTAATAGTAAATTATCCAATGTTTTATCTCGGTATTGGCGGTTTTGTG |
| | TAGATGGTGGAGCTAGCTCTTATTACCCTCAATATACCGAACTTCAAATCCTCGGAC |
| | AACGTTTATCAAATGATGTCGCTAATACGCTGAAGGATTGATATTAAAGTCCTACGA |
| | TTACAAAGATAAACAGTTTCTCAAAGTTGATTTAAGCACTTTAAAACCTAACTAAA |
| | AATCTGAGATGAATAGTCCCAGATTTTTAGTCTTTTATAGGTTTTGATGACATAAAG |
| | CTAAATAATCGTTAGACTACCAGAAAGTGGCGC |
| SEQ ID NO: 19 | GGCTCCGGCGCTACCAACTTCTCCCTGCTGAAGCAGGCTGGCGATGTGGAGGAGA |
| Ribosomal | ATCCCGGCCCT |
| Shifting peptide- | |
| P2A, nt | |
| SEQ ID NO: 20 | GGTTCAGGTGAGGGCCGCGGCAGTCTGCTGACTTGCGGCGACGTCGAGGAGAAC |
| Ribosomal | CCCGGTCCT |
| Shifting peptide- | |
| T2A, nt | |
| SEQ ID NO: 21 | GGGAGTGGGCAATGCACTAACTATGCATTGCTTAAGTTGGCTGGTGATGTAGAATC |
| Ribosomal | TAATCCTGGGCCC |
| Shifting peptide- | |
| E2A, nt | |
| SEQ ID NO: 22 | GGCAGTGGGGTGAAGCAGACCCTCAATTTTGACTTGTTGAAACTCGCAGGCGAC |
| Ribosomal | GTAGAATCTAACCCCGGACCT |
| Shifting peptide- | |
| F2A, nt | |
| SEQ ID NO: 23 | GACGGATCGGGAGATCTCCCGATCCCCTATGGTGCACTCTCAGTACAATCTGCTCT |
| SWG-006, nt | GATGCCGCATAGTTAAGCCAGTATCTGCTCCCTGCTTGTGTGTTGGAGGTCGCTGA |
| | GTAGTGCGCGAGCAAAATTTAAGCTACAACAAGGCAAGGCTTGACCGACAATTGC |
| | ATGAAGAATCTGCTTAGGGTTAGGCGTTTTGCGCTGCTTCGCGATGTACGGGCCAG |
| | ATATACGCGTTGACATTGATTATTGACTAGTTATTAATAGTAATCAATTACGGGGTCA |
| | TTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCC |
| | CATAGTAACGCCAATAGGGACTTTCCATTGACGTCAATGGGTGGAGTATTTACGGT |
| | AAACTGCCCACTTGGCAGTACATCAAGTGTATCATATGCCAAGTACGCCCCCTATT |
| | GACGTCAATGACGGTAAATGGCCCGCCTGGCATTATGCCCAGTACATGACCTTATG |
| | GGACTTTCCTACTTGGCAGTACATCTACGTATTAGTCATCGCTATTACCATGGTGAT |
| | GCGGTTTTGGCAGTACATCAATGGGCGTGGATAGCGGTTTGACTCACGGGGATTTC |
| | CAAGTCTCCACCCCATTGACGTCAATGGGAGTTTGTTTTGGCACCAAAATCAACG |
| | GGACTTTCCAAAATGTCGTAACAACTCCGCCCCATTGACGCAAATGGGCGGTAGG |
| | CGTGTACGGTGGGAGGTCTATATAAGCAGAGCTCTCTGGCTAACTAGAGAACCCA |
| | CTGCTTACTGGCTTATCGAAATTAATACGACTCACTATAGGGAGACCCAAGCTGGC |
| | TAGCGCCGCCACCATGATCCACACCAATCTGAAGAAGAAGTTTAGCTGTTGCGTG |
| | CTGGTGTTCCTGCTGTTCGCTGTGATCTGCGTGTGGAAGGAGAAGAAGAAGGGCA |
| | GCTATTATGACTCCTTTAAGCTGCAGACCAAGGAGTTCCAGGTGCTGAAGTCCCTG |
| | GGCAAGCTGGCCATGGGCAGCGACTCCCAGTCCGTGAGCTCCTCCAGCACCCAG |
| | GACCCCCACCGGGGAAGGCAGACCCTGGGATCCCTGAGGGGCCTGGCTAAGGCC |
| | AAGCCTGAGGCTTCCTTCCAGGTGTGGAATAAGGATAGCTCCAGCAAGAACCTGA |
| | TCCCTAGGCTGCAGAAGATCTGGAAGAACTATCTGAGCATGAACAAGTATAAGGT |
| | GAGCTATAAGGGCCCTGGCCCTGGCATCAAGTTCTCCGCCGAGGCCCTGAGGTGC |
| | CACCTGAGAGATCACGTGAATGTGAGCATGGTGGAGGTGACCGACTTCCCTTTTA |
| | ACACCTCCGAGTGGGAGGGCTACCTGCCCAAGGAGAGCATCCGGACCAAGGCCG |
| | GCCCTTGGGGCAGATGTGCCGTGGTGTCCAGCGCCGGCTCCCTGAAGAGCTCCCA |
| | GCTGGGCCGGGAGATCGATGACCACGATGCTGTGCTGAGGTTCAATGGCGCTCCT |
| | ACCGCTAACTTTCAGCAGGATGTGGGCACCAAGACCACCATCCGGCTGATGAATA |
| | GCCAGCTGGTGACCACCGAGAAGCGGTTTCTGAAGGACTCCCTGTACAACGAGG |
| | GCATCCTGATCGTGTGGGACCCCTCCGTGTACCACAGCGACATCCCCAAGTGGTAT |
| | CAGAACCCCGACTACAACTTCTTCAATAATTACAAGACCTACCGGAAGCTGCACC |
| | CCAACCAGCCCTTTTATATCCTGAAGCCCCAGATGCCTTGGGAGCTGTGGGACATC |
| | CTGCAGGAGATCTCCCCCGAGGAGATCCAGCCTAACCCCCCTAGCAGCGGCATGC |
| | TGGGCATCATCATCATGATGACCCTGTGCGATCAGGTGGATATCTACGAGTTCCTGC |
| | CCAGCAAGCGGAAGACCGACGTGTGTTATTATTATCAGAAGTTCTTCGATTCCGCC |
| | TGCACCATGGGCGCCTATCACCCTCTGCTGTACGAGAAGAATCTGGTGAAGCACC |
| | TGAACCAGGGCACCGACGAGGACATCTATCTGCTGGGCAAGGCCACCCTGCCCGG |
| | CTTTAGGACCATCCACTGCGGCAGCGGCGCTACCAATTTCAGCCTGCTGAAGCAG |
| | GCTGGCGATGTGGAGGAGAACCCCGGCCCCATGAGGCTGCGGGAGCCATTGCTG |
| | AGCGGCTCCGCTGCCATGCCTGGCGCTTCTCTGCAGAGGGCTTGTAGGCTGCTGG |
| | TGGCCGTGTGCGCCCTGCACCTGGGAGTGACCCTGGTGTACTACCTGGCTGGCCG |
| | GGACCTGTCCCGGCTGCCTCAGTTGGTGGGCGTGTCCACCCCTCTGCAGGGCGGA |
| | AGCAATAGCGCCGCTGCCATCGGCCAGTCCTCCGGAGAGCTGCGGACCGGAGGC |
| | GCTAGGCCACCTCCACCTCTGGGCGCTTCCTCCCAGCCTCGGCCTGGAGGAGATA |
| | GCAGCCCCGTGGTGGATAGCGGCCCTGGCCCAGCTTCTAACCTGACCAGCGTGCC |
| | TGTGCCCCACACCACCGCCCTGAGCCTGCCTGCTTGCCCCGAGGAGTCCCCCCTG |
| | CTGGTGGGACCAATGCTGATCGAGTTCAATATGCCTGTGGATCTGGAGCTGGTGGC |
| | CAAGCAGAATCCCAATGTGAAGATGGGCGGCCGGTACGCTCCCCGGGATTGTGTG |
| | TCCCCTCACAAGGTGGCTATCATCATCCCTTTCCGGAACCGGCAGGAGCACCTGA |
| | AGTACTGGCTGTATTACCTGCACCCCGTGCTGCAGAGGCAGCAGCTGGACTATGG |
| | CATCTACGTGATCAATCAGGCTGGCGACACCATCTTCAATCGGGCTAAGCTGCTGA |
| | ACGTGGGCTTTCAGGAGGCTCTGAAGGACTACGACTACACCTGCTTTGTGTTCAG |
| | CGATGTGGACCTGATCCCCATGAACGACCACAACGCTTATAGGTGCTTCTCCCAGC |
| | CTAGGCACATCTCCGTGGCTATGGACAAGTTTGGCTTCTCCCTGCCCTACGTGCAG |
| | TATTTTGGCGGCGTGTCCGCCCTGAGCAAGCAGCAGTTTCTGACCATCAATGGCTT |
| | TCCTAACAATTACTGGGGCTGGGGCGGCGAGGATGATGACATCTTCAACCGGCTG |
| | GTGTTCAGGGGCATGTCCATCAGCAGGCCTAACGCTGTGGTGGGCCGGTGTAGGA |
| | TGATCAGGCACTCCCGGGACAAGAAGAATGAGCCCAATCCTCAGCGGTTTGACCG |
| | GATCGCCCACACCAAGGAGACCATGCTGTCCGACGGCCTGAATTCCCTGACCTAC |
| | CAGGTGCTGGACGTGCAGAGGTATCCTCTGTACACCCAGATCACCGTGGATATCGG |
| | CACCCCTTCCTGATGATGACTCGAGTCTAGAGGGCCCGTTTAAACCCGCTGATCAG |
| | CCTCGACTGTGCCTTCTAGTTGCCAGCCATCTGTTGTTTGCCCCTCCCCCGTGCCTT |
| | CCTTGACCCTGGAAGGTGCCACTCCCACTGTCCTTTCCTAATAAAATGAGGAAATT |
| | GCATCGCATTGTCTGAGTAGGTGTCATTCTATTCTGGGGGGTGGGGGGGGCAGG |
| | ACAGCAAGGGGGAGGATTGGGAAGACAATAGCAGGCATGCTGGGGATGCGGTGG |
| | GCTCTATGGCTTCTGAGGCGGAAAGAACCAGCTGGGGCTCTAGGGGGTATCCCCA |
| | CGCGCCCTGTAGCGGCGCATTAAGCGCGGCGGGTGTGGTGGTTACGCGCAGCGTG |
| | ACCGCTACACTTGCCAGCGCCCTAGCGCCCGCTCCTTTCGCTTTCTTCCCTTCCTTT |
| | CTCGCCACGTTCGCCGGCTTTCCCCGTCAAGCTCTAAATCGGGGGCTCCCTTTAGG |
| | GTTCCGATTTAGTGCTTTACGGCACCTCGACCCCAAAAAACTTGATTAGGGTGATG |
| | GTTCACGTAGTGGGCCATCGCCCTGATAGACGGTTTTTCGCCCTTTGACGTTGGAG |
| | TCCACGTTCTTTAATAGTGGACTCTTGTTCCAAACTGGAACAACACTCAACCCTAT |
| | CTCGGTCTATTCTTTTGATTTATAAGGGATTTTGCCGATTTCGGCCTATTGGTTAAAA |
| | AATGAGCTGATTTAACAAAAATTTAACGCGAATTAATTCTGTGGAATGTGTGTCAG |
| | TTAGGGTGTGGAAAGTCCCCAGGCTCCCCAGCAGGCAGAAGTATGCAAAGCATGC |
| | ATCTCAATTAGTCAGCAACCAGGTGTGGAAAGTCCCCAGGCTCCCCAGCAGGCAG |
| | AAGTATGCAAAGCATGCATCTCAATTAGTCAGCAACCATAGTCCCGCCCCTAACTC |
| | CGCCCATCCCGCCCCTAACTCCGCCCAGTTCCGCCCATTCTCCGCCCCATGGCTGA |
| | CTAATTTTTTTTATTTATGCAGAGGCCGAGGCCGCCTCTGCCTCTGAGCTATTCCAG |
| | AAGTAGTGAGGAGGCTTTTTTGGAGGCCTAGGCTTTTGCAAAAAGCTCCCGGGAG |
| | CTTGTATATCCATTTTCGGATCTGATCAAGAGACAGGATGAGGATCGTTTCGCATGA |
| | TTGAACAAGATGGATTGCACGCAGGTTCTCCGGCCGCTTGGGTGGAGAGGCTATT |
| | CGGCTATGACTGGGCACAACAGACAATCGGCTGCTCTGATGCCGCCGTGTTCCGG |
| | CTGTCAGCGCAGGGGCGCCCGGTTCTTTTTGTCAAGACCGACCTGTCCGGTGCCC |
| | TGAATGAACTGCAGGACGAGGCAGCGCGGCTATCGTGGCTGGCCACGACGGGCG |
| | TTCCTTGCGCAGCTGTGCTCGACGTTGTCACTGAAGCGGGAAGGGACTGGCTGCT |
| | ATTGGGCGAAGTGCCGGGGCAGGATCTCCTGTCATCTCACCTTGCTCCTGCCGAG |
| | AAAGTATCCATCATGGCTGATGCAATGCGGCGGCTGCATACGCTTGATCCGGCTAC |
| | CTGCCCATTCGACCACCAAGCGAAACATCGCATCGAGCGAGCACGTACTCGGATG |
| | GAAGCCGGTCTTGTCGATCAGGATGATCTGGACGAAGAGCATCAGGGGCTCGCGC |
| | CAGCCGAACTGTTCGCCAGGCTCAAGGCGCGCATGCCCGACGGCGAGGATCTCGT |
| | CGTGACCCATGGCGATGCCTGCTTGCCGAATATCATGGTGGAAAATGGCCGCTTTT |
| | CTGGATTCATCGACTGTGGCCGGCTGGGTGTGGCGGACCGCTATCAGGACATAGC |
| | GTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTT |
| | CTTGACGAGTTCTTCTGAGCGGGACTCTGGGGTTCGAAATGACCGACCAAGCGAC |
| | GCCCAACCTGCCATCACGAGATTTCGATTCCACCGCCGCCTTCTATGAAAGGTTGG |
| | GCTTCGGAATCGTTTTCCGGGACGCCGGCTGGATGATCCTCCAGCGCGGGGATCT |
| | CATGCTGGAGTTCTTCGCCCACCCCAACTTGTTTATTGCAGCTTATAATGGTTACAA |
| | ATAAAGCAATAGCATCACAAATTTCACAAATAAAGCATTTTTTTCACTGCATTCTAG |
| | TTGTGGTTTGTCCAAACTCATCAATGTATCTTATCATGTCTGTATACCGTCGACCTCT |
| | AGCTAGAGCTTGGCGTAATCATGGTCATAGCTGTTTCCTGTGTGAAATTGTTATCCG |
| | CTCACAATTCCACACAACATACGAGCCGGAAGCATAAAGTGTAAAGCCTGGGGTG |
| | CCTAATGAGTGAGCTAACTCACATTAATTGCGTTGCGCTCACTGCCCGCTTTCCAG |
| | TCGGGAAACCTGTCGTGCCAGCTGCATTAATGAATCGGCCAACGCGCGGGGAGAG |
| | GCGGTTTGCGTATTGGGCGCTCTTCCGCTTCCTCGCTCACTGACTCGCTGCGCTCG |
| | GTCGTTCGGCTGCGGCGAGCGGTATCAGCTCACTCAAAGGCGGTAATACGGTTATC |
| | CACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCC |
| | CCTGACGAGCATCACAAAAATCGACGCTCAAGTCAGAGGTGGCGAAACCCGACA |
| | GGACTATAAAGATACCAGGCGTTTCCCCCTGGAAGCTCCCTCGTGCGCTCTCCTGT |
| | TCCGACCCTGCCGCTTACCGGATACCTGTCCGCCTTTCTCCCTTCGGGAAGCGTGG |
| | CGCTTTCTCATAGCTCACGCTGTAGGTATCTCAGTTCGGTGTAGGTCGTTCGCTCC |
| | AAGCTGGGCTGTGTGCACGAACCCCCCGTTCAGCCCGACCGCTGCGCCTTATCCG |
| | GTAACTATCGTCTTGAGTCCAACCCGGTAAGACACGACTTATCGCCACTGGCAGC |
| | AGCCACTGGTAACAGGATTAGCAGAGCGAGGTATGTAGGCGGTGCTACAGAGTTC |
| | TTGAAGTGGTGGCCTAACTACGGCTACACTAGAAGAACAGTATTTGGTATCTGCGC |
| | TCTGCTGAAGCCAGTTACCTTCGGAAAAAGAGTTGGTAGCTCTTGATCCGGCAAA |
| | CAAACCACCGCTGGTAGCGGTTTTTTTGTTTGCAAGCAGCAGATTACGCGCAGAA |
| | AAAAAGGATCTCAAGAAGATCCTTTGATCTTTTCTACGGGGTCTGACGCTCAGTG |
| | GAACGAAAACTCACGTTAAGGGATTTTGGTCATGAGATTATCAAAAAGGATCTTC |
| | ACCTAGATCCTTTTAAATTAAAAATGAAGTTTTAAATCAATCTAAAGTATATATGAG |
| | TAAACTTGGTCTGACAGTTACCAATGCTTAATCAGTGAGGCACCTATCTCAGCGAT |
| | CTGTCTATTTCGTTCATCCATAGTTGCCTGACTCCCCGTCGTGTAGATAACTACGAT |
| | ACGGGAGGGCTTACCATCTGGCCCCAGTGCTGCAATGATACCGCGAGACCCACGC |
| | TCACCGGCTCCAGATTTATCAGCAATAAACCAGCCAGCCGGAAGGGCCGAGCGCA |
| | GAAGTGGTCCTGCAACTTTATCCGCCTCCATCCAGTCTATTAATTGTTGCCGGGAA |
| | GCTAGAGTAAGTAGTTCGCCAGTTAATAGTTTGCGCAACGTTGTTGCCATTGCTAC |
| | AGGCATCGTGGTGTCACGCTCGTCGTTTGGTATGGCTTCATTCAGCTCCGGTTCCC |
| | AACGATCAAGGCGAGTTACATGATCCCCCATGTTGTGCAAAAAAGCGGTTAGCTC |
| | CTTCGGTCCTCCGATCGTTGTCAGAAGTAAGTTGGCCGCAGTGTTATCACTCATGG |
| | TTATGGCAGCACTGCATAATTCTCTTACTGTCATGCCATCCGTAAGATGCTTTTCTG |
| | TGACTGGTGAGTACTCAACCAAGTCATTCTGAGAATAGTGTATGCGGCGACCGAG |
| | TTGCTCTTGCCCGGCGTCAATACGGGATAATACCGCGCCACATAGCAGAACTTTAA |
| | AAGTGCTCATCATTGGAAAACGTTCTTCGGGGCGAAAACTCTCAAGGATCTTACC |
| | GCTGTTGAGATCCAGTTCGATGTAACCCACTCGTGCACCCAACTGATCTTCAGCAT |
| | CTTTTACTTTCACCAGCGTTTCTGGGTGAGCAAAAACAGGAAGGCAAAATGCCGC |
| | AAAAAAGGGAATAAGGGCGACACGGAAATGTTGAATACTCATACTCTTCCTTTTTC |
| | AATATTATTGAAGCATTTATCAGGGTTATTGTCTCATGAGCGGATACATATTTGAATG |
| | TATTTAGAAAAATAAACAAATAGGGGTTCCGCGCACATTTCCCCGAAAAGTGCCA |
| | CCTGACGTC |
| SEQ ID NO: 24 | GACGGATCGGGAGATCTCCCGATCCCCTATGGTGCACTCTCAGTACAATCTGCTCT |
| SWG-015, nt | GATGCCGCATAGTTAAGCCAGTATCTGCTCCCTGCTTGTGTGTTGGAGGTCGCTGA |
| | GTAGTGCGCGAGCAAAATTTAAGCTACAACAAGGCAAGGCTTGACCGACAATTGC |
| | ATGAAGAATCTGCTTAGGGTTAGGCGTTTTGCGCTGCTTCGCGATGTACGGGCCAG |
| | ATATACGCGTTGACATTGATTATTGACTAGTTATTAATAGTAATCAATTACGGGGTCA |
| | TTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCC |
| | CATAGTAACGCCAATAGGGACTTTCCATTGACGTCAATGGGTGGAGTATTTACGGT |
| | AAACTGCCCACTTGGCAGTACATCAAGTGTATCATATGCCAAGTACGCCCCCTATT |
| | GACGTCAATGACGGTAAATGGCCCGCCTGGCATTATGCCCAGTACATGACCTTATG |
| | GGACTTTCCTACTTGGCAGTACATCTACGTATTAGTCATCGCTATTACCATGGTGAT |
| | GCGGTTTTGGCAGTACATCAATGGGCGTGGATAGCGGTTTGACTCACGGGGATTTC |
| | CAAGTCTCCACCCCATTGACGTCAATGGGAGTTTGTTTTGGCACCAAAATCAACG |
| | GGACTTTCCAAAATGTCGTAACAACTCCGCCCCATTGACGCAAATGGGCGGTAGG |
| | CGTGTACGGTGGGAGGTCTATATAAGCAGAGCTCTCTGGCTAACTAGAGAACCCA |
| | CTGCTTACTGGCTTATCGAAATTAATACGACTCACTATAGGGAGACCCAAGCTGGC |
| | TAGCGCCGCCACCATGATCCACACCAACCTGAAGAAGAAGTTCTCCTGCTGTGTG |
| | CTGGTGTTTCTGCTGTTCGCTGTGATCTGCGTGTGGAAGGAGAAGAAGAAGGGCA |
| | GCTATTATGACAGCTTTAAGCTGCAGACCAAGGAGTTTCAGGTGCTGAAGTCCCT |
| | GGGCAAGCTGGCCATGGGCTCCGACTCCCAGTCCGTGTCCAGCTCCAGCACCCAG |
| | GATCCTCACCGGGGCAGGCAGACCCTGGGCTCTCTGAGAGGCCTGGCTAAGGCTA |
| | AGCCCGGCGGCGGAGGCAGCGGAGGAGGAGGATCTGGCGGCGGAGGATCCAATG |
| | TGGTGGCCCCTTCCCTGGAGGTGTATGTGGACCACGCCTCCCTGCCCACCCTGCA |
| | GCAGCTGATGGATATCATCAAGAGCGAGGAGGAGAATCCTACCGCCCAGCGGTAT |
| | ATCGCCTGGGGCCGGATCGTGCCTACCGACGAGCAGATGAAGGAGCTGAACATCA |
| | CCTCCTTCGCTCTGATCAACAATCACACCCCCGCCGACCTGGTGCAGGAGATCGT |
| | GAAGCAGGCCCAGACCAAGCACCGGCTGAACGTGAAGCTGTCCTCCAACACCGC |
| | CCACTCCTTTGACAACCTGGTGCCTATCCTGAAGGAGCTGAATTCCTTTAATAACG |
| | TGACCGTGACCAACATCGACCTGTACGACGACGGCTCCATGGAGTATGTGAACCT |
| | GTATAACTGGAGGGACACCCTGAATAAGACCGACAATCTGAAGATCGGCAAGGAC |
| | TATCTGGAGGACGTGATCAATGGCATCAATGAGGACACCTCCAACACCGGCACCA |
| | GCAGCGTGTATAACTGGCAGAAGCTGTACCCTGCTAATTACCACTTTCTGAGGAAG |
| | GATTATCTGACCCTGGAGCCTTCCCTGCACGAGCTGAGGGACTACATCGGCGATAG |
| | CCTGAAGCAGATGCAGTGGGATGGCTTTAAGAAGTTCAACAGCAAGCAGCAGGA |
| | GCTGTTTCTGAGCATCGTGAATTTTGACAAGCAGAAGCTGCAGAACGAGTATAAC |
| | TCCTCCAATCTGCCTAACTTTGTGTTCACCGGCACCACCGTGTGGGGCGGCAATCA |
| | CGAGCGGGAGTACTATGCCAAGCAGCAGATCAACGTGATCAATAATGCCATCAATG |
| | AGAGCAGCCCCCACTACCTGGGCAATAGCTACGATCTGTTCTTCAAGGGCCACCC |
| | TGGCGGCGGCATCATCAACACCCTGATCATGCAGAACTATCCTTCCATGGTGGATA |
| | TCCCTAGCAAGATCTCCTTCGAGGTGCTGATGATGACCGACATGCTGCCCGACGCC |
| | GTGGCTGGCATCGCCTCTAGCCTGTATTTTACCATCCCCGCTGAGAAGATCAAGTT |
| | CATCGTGTTTACCAGCACCGAGACCATCACCGACCGGGAGACCGCCCTGAGGTCC |
| | CCACTGGTGCAGGTGATGATCAAGCTGGGCATCGTGAAGGAGGAGAACGTGCTG |
| | TTTTGGGCCGACCTGCCCAACTGTGAGACCGGCGTGTGTGGCTCCGGCGCTACCA |
| | ACTTCTCCCTGCTGAAGCAGGCTGGCGATGTGGAGGAGAATCCCGGCCCTATGCG |
| | GCTGCGGGAGCCCCTGTTGTCCGGCTCTGCCGCTATGCCCGGCGCTTCCCTGCAG |
| | AGAGCCTGTCGGCTGCTGGTGGCTGTGTGCGCTCTGCACCTGGGCGTGACCCTGG |
| | TGTACTATCTGGCTGGCCGGGATCTGAGCCGGCTGCCTCAGCTGGTGGGCGTGAG |
| | CACCCCCCTGCAGGGAGGATCCAACTCCGCCGCTGCCATCGGCCAGTCCTCCGGA |
| | GAGCTGCGGACCGGAGGCGCTAGGCCTCCACCACCACTGGGCGCTTCTTCCCAGC |
| | CTCGGCCCGGAGGAGATAGCAGCCCCGTGGTGGACTCCGGCCCTGGACCTGCTTC |
| | CAACCTGACCAGCGTGCCCGTGCCTCACACCACCGCTCTGTCCCTGCCCGCCTGT |
| | CCCGAGGAGTCCCCTCTGCTGGTGGGCCCTATGCTGATCGAGTTTAATATGCCTGT |
| | GGACCTGGAGCTGGTGGCTAAGCAGAATCCTAACGTGAAGATGGGCGGCAGGTA |
| | CGCTCCCAGGGACTGCGTGAGCCCTCACAAGGTGGCCATCATCATCCCTTTCCGG |
| | AACCGGCAGGAGCACCTGAAGTACTGGCTGTACTACCTGCACCCTGTGCTGCAGA |
| | GGCAGCAGCTGGACTATGGCATCTACGTGATCAACCAGGCCGGCGACACCATCTT |
| | TAACCGGGCCAAGCTGCTGAATGTGGGCTTTCAGGAGGCCCTGAAGGATTATGAC |
| | TACACCTGCTTTGTGTTTAGCGATGTGGATCTGATCCCTATGAACGATCACAACGC |
| | CTACCGGTGCTTCAGCCAGCCTCGGCACATCAGCGTGGCCATGGACAAGTTCGGC |
| | TTCTCCCTGCCTTACGTGCAGTACTTCGGCGGCGTGTCCGCCCTGTCCAAGCAGCA |
| | GTTCCTGACCATCAACGGCTTTCCTAATAACTACTGGGGCTGGGGCGGCGAGGAT |
| | GATGACATCTTTAATCGGCTGGTGTTTAGGGGCATGAGCATCAGCCGGCCTAACGC |
| | CGTGGTGGGCAGGTGCAGGATGATCAGGCACTCCCGGGACAAGAAGAACGAGCC |
| | TAATCCTCAGAGGTTTGACAGGATCGCTCACACCAAGGAGACCATGCTGAGCGAC |
| | GGCCTGAATAGCCTGACCTACCAGGTGCTGGACGTGCAGAGGTACCCTCTGTATA |
| | CCCAGATCACCGTGGATATCGGCACCCCTAGCTGACTCGAGTCTAGAGGGCCCGT |
| | TTAAACCCGCTGATCAGCCTCGACTGTGCCTTCTAGTTGCCAGCCATCTGTTGTTT |
| | GCCCCTCCCCCGTGCCTTCCTTGACCCTGGAAGGTGCCACTCCCACTGTCCTTTCC |
| | TAATAAAATGAGGAAATTGCATCGCATTGTCTGAGTAGGTGTCATTCTATTCTGGGG |
| | GGTGGGGGGGGCAGGACAGCAAGGGGGAGGATTGGGAAGACAATAGCAGGCAT |
| | GCTGGGGATGCGGTGGGCTCTATGGCTTCTGAGGCGGAAAGAACCAGCTGGGGCT |
| | CTAGGGGGTATCCCCACGCGCCCTGTAGCGGCGCATTAAGCGCGGCGGGTGTGGT |
| | GGTTACGCGCAGCGTGACCGCTACACTTGCCAGCGCCCTAGCGCCCGCTCCTTTC |
| | GCTTTCTTCCCTTCCTTTCTCGCCACGTTCGCCGGCTTTCCCCGTCAAGCTCTAAAT |
| | CGGGGGCTCCCTTTAGGGTTCCGATTTAGTGCTTTACGGCACCTCGACCCCAAAAA |
| | ACTTGATTAGGGTGATGGTTCACGTAGTGGGCCATCGCCCTGATAGACGGTTTTTC |
| | GCCCTTTGACGTTGGAGTCCACGTTCTTTAATAGTGGACTCTTGTTCCAAACTGGA |
| | ACAACACTCAACCCTATCTCGGTCTATTCTTTTGATTTATAAGGGATTTTGCCGATT |
| | TCGGCCTATTGGTTAAAAAATGAGCTGATTTAACAAAAATTTAACGCGAATTAATT |
| | CTGTGGAATGTGTGTCAGTTAGGGTGTGGAAAGTCCCCAGGCTCCCCAGCAGGCA |
| | GAAGTATGCAAAGCATGCATCTCAATTAGTCAGCAACCAGGTGTGGAAAGTCCCC |
| | AGGCTCCCCAGCAGGCAGAAGTATGCAAAGCATGCATCTCAATTAGTCAGCAACC |
| | ATAGTCCCGCCCCTAACTCCGCCCATCCCGCCCCTAACTCCGCCCAGTTCCGCCCA |
| | TTCTCCGCCCCATGGCTGACTAATTTTTTTTATTTATGCAGAGGCCGAGGCCGCCTC |
| | TGCCTCTGAGCTATTCCAGAAGTAGTGAGGAGGCTTTTTTGGAGGCCTAGGCTTTT |
| | GCAAAAAGCTCCCGGGAGCTTGTATATCCATTTTCGGATCTGATCAAGAGACAGG |
| | ATGAGGATCGTTTCGCATGATTGAACAAGATGGATTGCACGCAGGTTCTCCGGCCG |
| | CTTGGGTGGAGAGGCTATTCGGCTATGACTGGGCACAACAGACAATCGGCTGCTC |
| | TGATGCCGCCGTGTTCCGGCTGTCAGCGCAGGGGCGCCCGGTTCTTTTTGTCAAG |
| | ACCGACCTGTCCGGTGCCCTGAATGAACTGCAGGACGAGGCAGCGCGGCTATCGT |
| | GGCTGGCCACGACGGGCGTTCCTTGCGCAGCTGTGCTCGACGTTGTCACTGAAGC |
| | GGGAAGGGACTGGCTGCTATTGGGCGAAGTGCCGGGGCAGGATCTCCTGTCATCT |
| | CACCTTGCTCCTGCCGAGAAAGTATCCATCATGGCTGATGCAATGCGGCGGCTGCA |
| | TACGCTTGATCCGGCTACCTGCCCATTCGACCACCAAGCGAAACATCGCATCGAGC |
| | GAGCACGTACTCGGATGGAAGCCGGTCTTGTCGATCAGGATGATCTGGACGAAGA |
| | GCATCAGGGGCTCGCGCCAGCCGAACTGTTCGCCAGGCTCAAGGCGCGCATGCCC |
| | GACGGCGAGGATCTCGTCGTGACCCATGGCGATGCCTGCTTGCCGAATATCATGGT |
| | GGAAAATGGCCGCTTTTCTGGATTCATCGACTGTGGCCGGCTGGGTGTGGCGGAC |
| | CGCTATCAGGACATAGCGTTGGCTACCCGTGATATTGCTGAAGAGCTTGGCGGCGA |
| | ATGGGCTGACCGCTTCCTCGTGCTTTACGGTATCGCCGCTCCCGATTCGCAGCGCA |
| | TCGCCTTCTATCGCCTTCTTGACGAGTTCTTCTGAGCGGGACTCTGGGGTTCGAAA |
| | TGACCGACCAAGCGACGCCCAACCTGCCATCACGAGATTTCGATTCCACCGCCGC |
| | CTTCTATGAAAGGTTGGGCTTCGGAATCGTTTTCCGGGACGCCGGCTGGATGATCC |
| | TCCAGCGCGGGGATCTCATGCTGGAGTTCTTCGCCCACCCCAACTTGTTTATTGCA |
| | GCTTATAATGGTTACAAATAAAGCAATAGCATCACAAATTTCACAAATAAAGCATTT |
| | TTTTCACTGCATTCTAGTTGTGGTTTGTCCAAACTCATCAATGTATCTTATCATGTCT |
| | GTATACCGTCGACCTCTAGCTAGAGCTTGGCGTAATCATGGTCATAGCTGTTTCCTG |
| | TGTGAAATTGTTATCCGCTCACAATTCCACACAACATACGAGCCGGAAGCATAAAG |
| | TGTAAAGCCTGGGGTGCCTAATGAGTGAGCTAACTCACATTAATTGCGTTGCGCTC |
| | ACTGCCCGCTTTCCAGTCGGGAAACCTGTCGTGCCAGCTGCATTAATGAATCGGCC |
| | AACGCGCGGGGAGAGGCGGTTTGCGTATTGGGCGCTCTTCCGCTTCCTCGCTCAC |
| | TGACTCGCTGCGCTCGGTCGTTCGGCTGCGGCGAGCGGTATCAGCTCACTCAAAG |
| | GCGGTAATACGGTTATCCACAGAATCAGGGGATAACGCAGGAAAGAACATGTGAG |
| | CAAAAGGCCAGCAAAAGGCCAGGAACCGTAAAAAGGCCGCGTTGCTGGCGTTTT |
| | TCCATAGGCTCCGCCCCCCTGACGAGCATCACAAAAATCGACGCTCAAGTCAGAG |
| | GTGGCGAAACCCGACAGGACTATAAAGATACCAGGCGTTTCCCCCTGGAAGCTCC |
| | CTCGTGCGCTCTCCTGTTCCGACCCTGCCGCTTACCGGATACCTGTCCGCCTTTCT |
| | CCCTTCGGGAAGCGTGGCGCTTTCTCATAGCTCACGCTGTAGGTATCTCAGTTCGG |
| | TGTAGGTCGTTCGCTCCAAGCTGGGCTGTGTGCACGAACCCCCCGTTCAGCCCGA |
| | CCGCTGCGCCTTATCCGGTAACTATCGTCTTGAGTCCAACCCGGTAAGACACGACT |
| | TATCGCCACTGGCAGCAGCCACTGGTAACAGGATTAGCAGAGCGAGGTATGTAGG |
| | CGGTGCTACAGAGTTCTTGAAGTGGTGGCCTAACTACGGCTACACTAGAAGAACA |
| | GTATTTGGTATCTGCGCTCTGCTGAAGCCAGTTACCTTCGGAAAAAGAGTTGGTAG |
| | CTCTTGATCCGGCAAACAAACCACCGCTGGTAGCGGTTTTTTTGTTTGCAAGCAG |
| | CAGATTACGCGCAGAAAAAAAGGATCTCAAGAAGATCCTTTGATCTTTTCTACGG |
| | GGTCTGACGCTCAGTGGAACGAAAACTCACGTTAAGGGATTTTGGTCATGAGATT |
| | ATCAAAAAGGATCTTCACCTAGATCCTTTTAAATTAAAAATGAAGTTTTAAATCAAT |
| | CTAAAGTATATATGAGTAAACTTGGTCTGACAGTTACCAATGCTTAATCAGTGAGG |
| | CACCTATCTCAGCGATCTGTCTATTTCGTTCATCCATAGTTGCCTGACTCCCCGTCG |
| | TGTAGATAACTACGATACGGGAGGGCTTACCATCTGGCCCCAGTGCTGCAATGATA |
| | CCGCGAGACCCACGCTCACCGGCTCCAGATTTATCAGCAATAAACCAGCCAGCCG |
| | GAAGGGCCGAGCGCAGAAGTGGTCCTGCAACTTTATCCGCCTCCATCCAGTCTATT |
| | AATTGTTGCCGGGAAGCTAGAGTAAGTAGTTCGCCAGTTAATAGTTTGCGCAACGT |
| | TGTTGCCATTGCTACAGGCATCGTGGTGTCACGCTCGTCGTTTGGTATGGCTTCATT |
| | CAGCTCCGGTTCCCAACGATCAAGGCGAGTTACATGATCCCCCATGTTGTGCAAA |
| | AAAGCGGTTAGCTCCTTCGGTCCTCCGATCGTTGTCAGAAGTAAGTTGGCCGCAG |
| | TGTTATCACTCATGGTTATGGCAGCACTGCATAATTCTCTTACTGTCATGCCATCCG |
| | TAAGATGCTTTTCTGTGACTGGTGAGTACTCAACCAAGTCATTCTGAGAATAGTGT |
| | ATGCGGCGACCGAGTTGCTCTTGCCCGGCGTCAATACGGGATAATACCGCGCCAC |
| | ATAGCAGAACTTTAAAAGTGCTCATCATTGGAAAACGTTCTTCGGGGCGAAAACT |
| | CTCAAGGATCTTACCGCTGTTGAGATCCAGTTCGATGTAACCCACTCGTGCACCCA |
| | ACTGATCTTCAGCATCTTTTACTTTCACCAGCGTTTCTGGGTGAGCAAAAACAGGA |
| | AGGCAAAATGCCGCAAAAAAGGGAATAAGGGCGACACGGAAATGTTGAATACTC |
| | ATACTCTTCCTTTTTCAATATTATTGAAGCATTTATCAGGGTTATTGTCTCATGAGCG |
| | GATACATATTTGAATGTATTTAGAAAAATAAACAAATAGGGGTTCCGCGCACATTTC |
| | CCCGAAAAGTGCCACCTGACGTC |
| SEQ ID NO: 25 | GACGGATCGGGAGATCTCCCGATCCCCTATGGTGCACTCTCAGTACAATCTGCTCG |
| ST6GAL1 | ATGCCGCATAGTTAAGCCAGTATCTGCTCCCTGCTTGTGTGTTGGAGGTCGCTGAG |
| expression | TAGTGCGCGAGCAAAATTTAAGCTACAACAAGGCAAGGCTTGACCGACAATTGCA |
| vector | TGAAGAATCTGCTTAGGGTTAGGCGTTTTGCGCTGCTTCGCGATGTACGGGCCAGA |
| | TATACGCGTTGACATTGATTATTGACTAGTTATTAATAGTAATCAATTACGGGGTCAT |
| | TAGTTCATAGCCCATATATGGAGTTCCGCGTTACATAACTTACGGTAAATGGCCCGC |
| | CTGGCTGACCGCCCAACGACCCCCGCCCATTGACGTCAATAATGACGTATGTTCCC |
| | ATAGTAACGCCAATAGGGACTTTCCATTGACGTCAATGGGTGGAGTATTTACGGTA |
| | AACTGCCCACTTGGCAGTACATCAAGTGTATCATATGCCAAGTACGCCCCCTATTG |
| | ACGTCAATGACGGTAAATGGCCCGCCTGGCATTATGCCCAGTACATGACCTTATGG |
| | GACTTTCCTACTTGGCAGTACATCTACGTATTAGTCATCGCTATTACCATGGTGATG |
| | CGGTTTTGGCAGTACATCAATGGGCGTGGATAGCGGTTTGACTCACGGGGATTTCC |
| | AAGTCTCCACCCCATTGACGTCAATGGGAGTTTGTTTTGGCACCAAAATCAACGG |
| | GACTTTCCAAAATGTCGTAACAACTCCGCCCCATTGACGCAAATGGGCGGTAGGC |
| | GTGTACGGTGGGAGGTCTATATAAGCAGAGCTCTCTGGCTAACTAGAGAACCCAC |
| | TGCTTACTGGCTTATCGAAATTAATACGACTCACTATAGGGAGACCCAAGCTGGCT |
| | AGCGCCGCCACCATGATCCACACCAATCTGAAGAAGAAGTTTAGCTGTTGCGTGC |
| | TGGTGTTCCTGCTGTTCGCTGTGATCTGCGTGTGGAAGGAGAAGAAGAAGGGCA |
| | GCTATTATGACTCCTTTAAGCTGCAGACCAAGGAGTTCCAGGTGCTGAAGTCCCTG |
| | GGCAAGCTGGCCATGGGCAGCGACTCCCAGTCCGTGAGCTCCTCCAGCACCCAG |
| | GACCCCCACCGGGGAAGGCAGACCCTGGGATCCCTGAGGGGCCTGGCTAAGGCC |
| | AAGCCTGAGGCTTCCTTCCAGGTGTGGAATAAGGATAGCTCCAGCAAGAACCTGA |
| | TCCCTAGGCTGCAGAAGATCTGGAAGAACTATCTGAGCATGAACAAGTATAAGGT |
| | GAGCTATAAGGGCCCTGGCCCTGGCATCAAGTTCTCCGCCGAGGCCCTGAGGTGC |
| | CACCTGAGAGATCACGTGAATGTGAGCATGGTGGAGGTGACCGACTTCCCTTTTA |
| | ACACCTCCGAGTGGGAGGGCTACCTGCCCAAGGAGAGCATCCGGACCAAGGCCG |
| | GCCCTTGGGGCAGATGTGCCGTGGTGTCCAGCGCCGGCTCCCTGAAGAGCTCCCA |
| | GCTGGGCCGGGAGATCGATGACCACGATGCTGTGCTGAGGTTCAATGGCGCTCCT |
| | ACCGCTAACTTTCAGCAGGATGTGGGCACCAAGACCACCATCCGGCTGATGAATA |
| | GCCAGCTGGTGACCACCGAGAAGCGGTTTCTGAAGGACTCCCTGTACAACGAGG |
| | GCATCCTGATCGTGTGGGACCCCTCCGTGTACCACAGCGACATCCCCAAGTGGTAT |
| | CAGAACCCCGACTACAACTTCTTCAATAATTACAAGACCTACCGGAAGCTGCACC |
| | CCAACCAGCCCTTTTATATCCTGAAGCCCCAGATGCCTTGGGAGCTGTGGGACATC |
| | CTGCAGGAGATCTCCCCCGAGGAGATCCAGCCTAACCCCCCTAGCAGCGGCATGC |
| | TGGGCATCATCATCATGATGACCCTGTGCGATCAGGTGGATATCTACGAGTTCCTGC |
| | CCAGCAAGCGGAAGACCGACGTGTGTTATTATTATCAGAAGTTCTTCGATTCCGCC |
| | TGCACCATGGGCGCCTATCACCCTCTGCTGTACGAGAAGAATCTGGTGAAGCACC |
| | TGAACCAGGGCACCGACGAGGACATCTATCTGCTGGGCAAGGCCACCCTGCCCGG |
| | CTTTAGGACCATCCACTGCTGACTCGAGTCTAGAGGGCCCGTTTAAACCCGCTGAT |
| | CAGCCTCGACTGTGCCTTCTAGTTGCCAGCCATCTGTTGTTTGCCCCTCCCCCGTG |
| | CCTTCCTTGACCCTGGAAGGTGCCACTCCCACTGTCCTTTCCTAATAAAATGAGGA |
| | AATTGCATCGCATTGTCTGAGTAGGTGTCATTCTATTCTGGGGGGTGGGGGGGGC |
| | AGGACAGCAAGGGGGAGGATTGGGAAGACAATAGCAGGCATGCTGGGGATGCGG |
| | TGGGCTCTATGGCTTCTGAGGCGGAAAGAACCAGCTGGGGCTCTAGGGGGTATCC |
| | CCACGCGCCCTGTAGCGGCGCATTAAGCGCGGCGGGTGTGGTGGTTACGCGCAGC |
| | GTGACCGCTACACTTGCCAGCGCCCTAGCGCCCGCTCCTTTCGCTTTCTTCCCTTC |
| | CTTTCTCGCCACGTTCGCCGGCTTTCCCCGTCAAGCTCTAAATCGGGGGCTCCCTT |
| | TAGGGTTCCGATTTAGTGCTTTACGGCACCTCGACCCCAAAAAACTTGATTAGGGT |
| | GATGGTTCACGTAGTGGGCCATCGCCCTGATAGACGGTTTTTCGCCCTTTGACGTT |
| | GGAGTCCACGTTCTTTAATAGTGGACTCTTGTTCCAAACTGGAACAACACTCAAC |
| | CCTATCTCGGTCTATTCTTTTGATTTATAAGGGATTTTGCCGATTTCGGCCTATTGGT |
| | TAAAAAATGAGCTGATTTAACAAAAATTTAACGCGAATTAATTCTGTGGAATGTGT |
| | GTCAGTTAGGGTGTGGAAAGTCCCCAGGCTCCCCAGCAGGCAGAAGTATGCAAA |
| | GCATGCATCTCAATTAGTCAGCAACCAGGTGTGGAAAGTCCCCAGGCTCCCCAGC |
| | AGGCAGAAGTATGCAAAGCATGCATCTCAATTAGTCAGCAACCATAGTCCCGCCC |
| | CTAACTCCGCCCATCCCGCCCCTAACTCCGCCCAGTTCCGCCCATTCTCCGCCCCA |
| | TGGCTGACTAATTTTTTTTATTTATGCAGAGGCCGAGGCCGCCTCTGCCTCTGAGCT |
| | ATTCCAGAAGTAGTGAGGAGGCTTTTTTGGAGGCCTAGGCTTTTGCAAAAAGCTC |
| | CCGGGAGCTTGTATATCCATTTTCGGATCTGATCAAGAGACAGGATGAGGATCGTT |
| | TCGCATGATTGAACAAGATGGATTGCACGCAGGTTCTCCGGCCGCTTGGGTGGAG |
| | AGGCTATTCGGCTATGACTGGGCACAACAGACAATCGGCTGCTCTGATGCCGCCG |
| | TGTTCCGGCTGTCAGCGCAGGGGCGCCCGGTTCTTTTTGTCAAGACCGACCTGTC |
| | CGGTGCCCTGAATGAACTGCAGGACGAGGCAGCGCGGCTATCGTGGCTGGCCAC |
| | GACGGGCGTTCCTTGCGCAGCTGTGCTCGACGTTGTCACTGAAGCGGGAAGGGA |
| | CTGGCTGCTATTGGGCGAAGTGCCGGGGCAGGATCTCCTGTCATCTCACCTTGCTC |
| | CTGCCGAGAAAGTATCCATCATGGCTGATGCAATGCGGCGGCTGCATACGCTTGAT |
| | CCGGCTACCTGCCCATTCGACCACCAAGCGAAACATCGCATCGAGCGAGCACGTA |
| | CTCGGATGGAAGCCGGTCTTGTCGATCAGGATGATCTGGACGAAGAGCATCAGGG |
| | GCTCGCGCCAGCCGAACTGTTCGCCAGGCTCAAGGCGCGCATGCCCGACGGCGA |
| | GGATCTCGTCGTGACCCATGGCGATGCCTGCTTGCCGAATATCATGGTGGAAAATG |
| | GCCGCTTTTCTGGATTCATCGACTGTGGCCGGCTGGGTGTGGCGGACCGCTATCAG |
| | GACATAGCGTTGGCTACCCGTGATATTGCTGAAGAGCTTGGCGGCGAATGGGCTG |
| | ACCGCTTCCTCGTGCTTTACGGTATCGCCGCTCCCGATTCGCAGCGCATCGCCTTC |
| | TATCGCCTTCTTGACGAGTTCTTCTGAGCGGGACTCTGGGGTTCGAAATGACCGAC |
| | CAAGCGACGCCCAACCTGCCATCACGAGATTTCGATTCCACCGCCGCCTTCTATGA |
| | AAGGTTGGGCTTCGGAATCGTTTTCCGGGACGCCGGCTGGATGATCCTCCAGCGC |
| | GGGGATCTCATGCTGGAGTTCTTCGCCCACCCCAACTTGTTTATTGCAGCTTATAAT |
| | GGTTACAAATAAAGCAATAGCATCACAAATTTCACAAATAAAGCATTTTTTTCACT |
| | GCATTCTAGTTGTGGTTTGTCCAAACTCATCAATGTATCTTATCATGTCTGTATACCG |
| | TCGACCTCTAGCTAGAGCTTGGCGTAATCATGGTCATAGCTGTTTCCTGTGTGAAA |
| | TTGTTATCCGCTCACAATTCCACACAACATACGAGCCGGAAGCATAAAGTGTAAAG |
| | CCTGGGGTGCCTAATGAGTGAGCTAACTCACATTAATTGCGTTGCGCTCACTGCCC |
| | GCTTTCCAGTCGGGAAACCTGTCGTGCCAGCTGCATTAATGAATCGGCCAACGCG |
| | CGGGGAGAGGCGGTTTGCGTATTGGGCGCTCTTCCGCTTCCTCGCTCACTGACTC |
| | GCTGCGCTCGGTCGTTCGGCTGCGGCGAGCGGTATCAGCTCACTCAAAGGCGGTA |
| | ATACGGTTATCCACAGAATCAGGGGATAACGCAGGAAAGAACATGTGAGCAAAAG |
| | GCCAGCAAAAGGCCAGGAACCGTAAAAAGGCCGCGTTGCTGGCGTTTTTCCATAG |
| | GCTCCGCCCCCCTGACGAGCATCACAAAAATCGACGCTCAAGTCAGAGGTGGCG |
| | AAACCCGACAGGACTATAAAGATACCAGGCGTTTCCCCCTGGAAGCTCCCTCGTG |
| | CGCTCTCCTGTTCCGACCCTGCCGCTTACCGGATACCTGTCCGCCTTTCTCCCTTCG |
| | GGAAGCGTGGCGCTTTCTCATAGCTCACGCTGTAGGTATCTCAGTTCGGTGTAGGT |
| | CGTTCGCTCCAAGCTGGGCTGTGTGCACGAACCCCCCGTTCAGCCCGACCGCTGC |
| | GCCTTATCCGGTAACTATCGTCTTGAGTCCAACCCGGTAAGACACGACTTATCGCC |
| | ACTGGCAGCAGCCACTGGTAACAGGATTAGCAGAGCGAGGTATGTAGGCGGTGCT |
| | ACAGAGTTCTTGAAGTGGTGGCCTAACTACGGCTACACTAGAAGAACAGTATTTG |
| | GTATCTGCGCTCTGCTGAAGCCAGTTACCTTCGGAAAAAGAGTTGGTAGCTCTTG |
| | ATCCGGCAAACAAACCACCGCTGGTAGCGGTTTTTTTGTTTGCAAGCAGCAGATT |
| | ACGCGCAGAAAAAAAGGATCTCAAGAAGATCCTTTGATCTTTTCTACGGGGTCTG |
| | ACGCTCAGTGGAACGAAAACTCACGTTAAGGGATTTTGGTCATGAGATTATCAAA |
| | AAGGATCTTCACCTAGATCCTTTTAAATTAAAAATGAAGTTTTAAATCAATCTAAAG |
| | TATATATGAGTAAACTTGGTCTGACAGTTACCAATGCTTAATCAGTGAGGCACCTAT |
| | CTCAGCGATCTGTCTATTTCGTTCATCCATAGTTGCCTGACTCCCCGTCGTGTAGAT |
| | AACTACGATACGGGAGGGCTTACCATCTGGCCCCAGTGCTGCAATGATACCGCGA |
| | GACCCACGCTCACCGGCTCCAGATTTATCAGCAATAAACCAGCCAGCCGGAAGGG |
| | CCGAGCGCAGAAGTGGTCCTGCAACTTTATCCGCCTCCATCCAGTCTATTAATTGT |
| | TGCCGGGAAGCTAGAGTAAGTAGTTCGCCAGTTAATAGTTTGCGCAACGTTGTTG |
| | CCATTGCTACAGGCATCGTGGTGTCACGCTCGTCGTTTGGTATGGCTTCATTCAGC |
| | TCCGGTTCCCAACGATCAAGGCGAGTTACATGATCCCCCATGTTGTGCAAAAAAG |
| | CGGTTAGCTCCTTCGGTCCTCCGATCGTTGTCAGAAGTAAGTTGGCCGCAGTGTTA |
| | TCACTCATGGTTATGGCAGCACTGCATAATTCTCTTACTGTCATGCCATCCGTAAGA |
| | TGCTTTTCTGTGACTGGTGAGTACTCAACCAAGTCATTCTGAGAATAGTGTATGCG |
| | GCGACCGAGTTGCTCTTGCCCGGCGTCAATACGGGATAATACCGCGCCACATAGCA |
| | GAACTTTAAAAGTGCTCATCATTGGAAAACGTTCTTCGGGGCGAAAACTCTCAAG |
| | GATCTTACCGCTGTTGAGATCCAGTTCGATGTAACCCACTCGTGCACCCAACTGAT |
| | CTTCAGCATCTTTTACTTTCACCAGCGTTTCTGGGTGAGCAAAAACAGGAAGGCA |
| | AAATGCCGCAAAAAAGGGAATAAGGGCGACACGGAAATGTTGAATACTCATACTC |
| | TTCCTTTTTCAATATTATTGAAGCATTTATCAGGGTTATTGTCTCATGAGCGGATACA |
| | TATTTGAATGTATTTAGAAAAATAAACAAATAGGGGTTCCGCGCACATTTCCCCGA |
| | AAAGTGCCACCTGACGTC |
| SEQ ID NO: 26 | GACGGATCGGGAGATCTCCCGATCCCCTATGGTGCACTCTCAGTACAATCTGCTCT |
| B4GALT1 | GATGCCGCATAGTTAAGCCAGTATCTGCTCCCTGCTTGTGTGTTGGAGGTCGCTGA |
| expression | GTAGTGCGCGAGCAAAATTTAAGCTACAACAAGGCAAGGCTTGACCGACAATTGC |
| vector | ATGAAGAATCTGCTTAGGGTTAGGCGTTTTGCGCTGCTTCGCGATGTACGGGCCAG |
| | ATATACGCGTTGACATTGATTATTGACTAGTTATTAATAGTAATCAATTACGGGGTCA |
| | TTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCC |
| | CATAGTAACGCCAATAGGGACTTTCCATTGACGTCAATGGGTGGAGTATTTACGGT |
| | AAACTGCCCACTTGGCAGTACATCAAGTGTATCATATGCCAAGTACGCCCCCTATT |
| | GACGTCAATGACGGTAAATGGCCCGCCTGGCATTATGCCCAGTACATGACCTTATG |
| | GGACTTTCCTACTTGGCAGTACATCTACGTATTAGTCATCGCTATTACCATGGTGAT |
| | GCGGTTTTGGCAGTACATCAATGGGCGTGGATAGCGGTTTGACTCACGGGGATTTC |
| | CAAGTCTCCACCCCATTGACGTCAATGGGAGTTTGTTTTGGCACCAAAATCAACG |
| | GGACTTTCCAAAATGTCGTAACAACTCCGCCCCATTGACGCAAATGGGCGGTAGG |
| | CGTGTACGGTGGGAGGTCTATATAAGCAGAGCTCTCTGGCTAACTAGAGAACCCA |
| | CTGCTTACTGGCTTATCGAAATTAATACGACTCACTATAGGGAGACCCAAGCTGGC |
| | TAGCGCCGCCACCATGAGGCTGCGGGAGCCATTGCTGAGCGGCTCCGCTGCCATG |
| | CCTGGCGCTTCTCTGCAGAGGGCTTGTAGGCTGCTGGTGGCCGTGTGCGCCCTGC |
| | ACCTGGGAGTGACCCTGGTGTACTACCTGGCTGGCCGGGACCTGTCCCGGCTGCC |
| | TCAGTTGGTGGGCGTGTCCACCCCTCTGCAGGGCGGAAGCAATAGCGCCGCTGCC |
| | ATCGGCCAGTCCTCCGGAGAGCTGCGGACCGGAGGCGCTAGGCCACCTCCACCTC |
| | TGGGCGCTTCCTCCCAGCCTCGGCCTGGAGGAGATAGCAGCCCCGTGGTGGATAG |
| | CGGCCCTGGCCCAGCTTCTAACCTGACCAGCGTGCCTGTGCCCCACACCACCGCC |
| | CTGAGCCTGCCTGCTTGCCCCGAGGAGTCCCCCCTGCTGGTGGGACCAATGCTGA |
| | TCGAGTTCAATATGCCTGTGGATCTGGAGCTGGTGGCCAAGCAGAATCCCAATGTG |
| | AAGATGGGCGGCCGGTACGCTCCCCGGGATTGTGTGTCCCCTCACAAGGTGGCTA |
| | TCATCATCCCTTTCCGGAACCGGCAGGAGCACCTGAAGTACTGGCTGTATTACCTG |
| | CACCCCGTGCTGCAGAGGCAGCAGCTGGACTATGGCATCTACGTGATCAATCAGG |
| | CTGGCGACACCATCTTCAATCGGGCTAAGCTGCTGAACGTGGGCTTTCAGGAGGC |
| | TCTGAAGGACTACGACTACACCTGCTTTGTGTTCAGCGATGTGGACCTGATCCCCA |
| | TGAACGACCACAACGCTTATAGGTGCTTCTCCCAGCCTAGGCACATCTCCGTGGCT |
| | ATGGACAAGTTTGGCTTCTCCCTGCCCTACGTGCAGTATTTTGGCGGCGTGTCCGC |
| | CCTGAGCAAGCAGCAGTTTCTGACCATCAATGGCTTTCCTAACAATTACTGGGGCT |
| | GGGGCGGCGAGGATGATGACATCTTCAACCGGCTGGTGTTCAGGGGCATGTCCAT |
| | CAGCAGGCCTAACGCTGTGGTGGGCCGGTGTAGGATGATCAGGCACTCCCGGGAC |
| | AAGAAGAATGAGCCCAATCCTCAGCGGTTTGACCGGATCGCCCACACCAAGGAG |
| | ACCATGCTGTCCGACGGCCTGAATTCCCTGACCTACCAGGTGCTGGACGTGCAGA |
| | GGTATCCTCTGTACACCCAGATCACCGTGGATATCGGCACCCCTTCCTGACTCGAG |
| | TCTAGAGGGCCCGTTTAAACCCGCTGATCAGCCTCGACTGTGCCTTCTAGTTGCCA |
| | GCCATCTGTTGTTTGCCCCTCCCCCGTGCCTTCCTTGACCCTGGAAGGTGCCACTC |
| | CCACTGTCCTTTCCTAATAAAATGAGGAAATTGCATCGCATTGTCTGAGTAGGTGT |
| | CATTCTATTCTGGGGGGTGGGGTGGGGCAGGACAGCAAGGGGGAGGATTGGGAA |
| | GACAATAGCAGGCATGCTGGGGATGCGGTGGGCTCTATGGCTTCTGAGGCGGAAA |
| | GAACCAGCTGGGGCTCTAGGGGGTATCCCCACGCGCCCTGTAGCGGCGCATTAAG |
| | CGCGGCGGGTGTGGTGGTTACGCGCAGCGTGACCGCTACACTTGCCAGCGCCCTA |
| | GCGCCCGCTCCTTTCGCTTTCTTCCCTTCCTTTCTCGCCACGTTCGCCGGCTTTCCC |
| | CGTCAAGCTCTAAATCGGGGGCTCCCTTTAGGGTTCCGATTTAGTGCTTTACGGCA |
| | CCTCGACCCCAAAAAACTTGATTAGGGTGATGGTTCACGTAGTGGGCCATCGCCC |
| | TGATAGACGGTTTTTCGCCCTTTGACGTTGGAGTCCACGTTCTTTAATAGTGGACT |
| | CTTGTTCCAAACTGGAACAACACTCAACCCTATCTCGGTCTATTCTTTTGATTTATA |
| | AGGGATTTTGCCGATTTCGGCCTATTGGTTAAAAAATGAGCTGATTTAACAAAAAT |
| | TTAACGCGAATTAATTCTGTGGAATGTGTGTCAGTTAGGGTGTGGAAAGTCCCCAG |
| | GCTCCCCAGCAGGCAGAAGTATGCAAAGCATGCATCTCAATTAGTCAGCAACCAG |
| | GTGTGGAAAGTCCCCAGGCTCCCCAGCAGGCAGAAGTATGCAAAGCATGCATCTC |
| | AATTAGTCAGCAACCATAGTCCCGCCCCTAACTCCGCCCATCCCGCCCCTAACTCC |
| | GCCCAGTTCCGCCCATTCTCCGCCCCATGGCTGACTAATTTTTTTTATTTATGCAGA |
| | GGCCGAGGCCGCCTCTGCCTCTGAGCTATTCCAGAAGTAGTGAGGAGGCTTTTTT |
| | GGAGGCCTAGGCTTTTGCAAAAAGCTCCCGGGAGCTTGTATATCCATTTTCGGATC |
| | TGATCAAGAGACAGGATGAGGATCGTTTCGCATGATTGAACAAGATGGATTGCAC |
| | GCAGGTTCTCCGGCCGCTTGGGTGGAGAGGCTATTCGGCTATGACTGGGCACAAC |
| | AGACAATCGGCTGCTCTGATGCCGCCGTGTTCCGGCTGTCAGCGCAGGGGCGCCC |
| | GGTTCTTTTTGTCAAGACCGACCTGTCCGGTGCCCTGAATGAACTGCAGGACGAG |
| | GCAGCGCGGCTATCGTGGCTGGCCACGACGGGCGTTCCTTGCGCAGCTGTGCTCG |
| | ACGTTGTCACTGAAGCGGGAAGGGACTGGCTGCTATTGGGCGAAGTGCCGGGGC |
| | AGGATCTCCTGTCATCTCACCTTGCTCCTGCCGAGAAAGTATCCATCATGGCTGAT |
| | GCAATGCGGCGGCTGCATACGCTTGATCCGGCTACCTGCCCATTCGACCACCAAGC |
| | GAAACATCGCATCGAGCGAGCACGTACTCGGATGGAAGCCGGTCTTGTCGATCAG |
| | GATGATCTGGACGAAGAGCATCAGGGGCTCGCGCCAGCCGAACTGTTCGCCAGG |
| | CTCAAGGCGCGCATGCCCGACGGCGAGGATCTCGTCGTGACCCATGGCGATGCCT |
| | GCTTGCCGAATATCATGGTGGAAAATGGCCGCTTTTCTGGATTCATCGACTGTGGC |
| | CGGCTGGGTGTGGCGGACCGCTATCAGGACATAGCGTTGGCTACCCGTGATATTGC |
| | TGAAGAGCTTGGCGGCGAATGGGCTGACCGCTTCCTCGTGCTTTACGGTATCGCC |
| | GCTCCCGATTCGCAGCGCATCGCCTTCTATCGCCTTCTTGACGAGTTCTTCTGAGC |
| | GGGACTCTGGGGTTCGAAATGACCGACCAAGCGACGCCCAACCTGCCATCACGA |
| | GATTTCGATTCCACCGCCGCCTTCTATGAAAGGTTGGGCTTCGGAATCGTTTTCCG |
| | GGACGCCGGCTGGATGATCCTCCAGCGCGGGGATCTCATGCTGGAGTTCTTCGCC |
| | CACCCCAACTTGTTTATTGCAGCTTATAATGGTTACAAATAAAGCAATAGCATCACA |
| | AATTTCACAAATAAAGCATTTTTTTCACTGCATTCTAGTTGTGGTTTGTCCAAACTC |
| | ATCAATGTATCTTATCATGTCTGTATACCGTCGACCTCTAGCTAGAGCTTGGCGTAA |
| | TCATGGTCATAGCTGTTTCCTGTGTGAAATTGTTATCCGCTCACAATTCCACACAAC |
| | ATACGAGCCGGAAGCATAAAGTGTAAAGCCTGGGGTGCCTAATGAGTGAGCTAAC |
| | TCACATTAATTGCGTTGCGCTCACTGCCCGCTTTCCAGTCGGGAAACCTGTCGTGC |
| | CAGCTGCATTAATGAATCGGCCAACGCGCGGGGAGAGGCGGTTTGCGTATTGGGC |
| | GCTCTTCCGCTTCCTCGCTCACTGACTCGCTGCGCTCGGTCGTTCGGCTGCGGCGA |
| | GCGGTATCAGCTCACTCAAAGGCGGTAATACGGTTATCCACAGAATCAGGGGATA |
| | ACGCAGGAAAGAACATGTGAGCAAAAGGCCAGCAAAAGGCCAGGAACCGTAAA |
| | AAGGCCGCGTTGCTGGCGTTTTTCCATAGGCTCCGCCCCCCTGACGAGCATCACA |
| | AAAATCGACGCTCAAGTCAGAGGTGGCGAAACCCGACAGGACTATAAAGATACC |
| | AGGCGTTTCCCCCTGGAAGCTCCCTCGTGCGCTCTCCTGTTCCGACCCTGCCGCTT |
| | ACCGGATACCTGTCCGCCTTTCTCCCTTCGGGAAGCGTGGCGCTTTCTCATAGCTC |
| | ACGCTGTAGGTATCTCAGTTCGGTGTAGGTCGTTCGCTCCAAGCTGGGCTGTGTGC |
| | ACGAACCCCCCGTTCAGCCCGACCGCTGCGCCTTATCCGGTAACTATCGTCTTGAG |
| | TCCAACCCGGTAAGACACGACTTATCGCCACTGGCAGCAGCCACTGGTAACAGGA |
| | TTAGCAGAGCGAGGTATGTAGGCGGTGCTACAGAGTTCTTGAAGTGGTGGCCTAA |
| | CTACGGCTACACTAGAAGAACAGTATTTGGTATCTGCGCTCTGCTGAAGCCAGTTA |
| | CCTTCGGAAAAAGAGTTGGTAGCTCTTGATCCGGCAAACAAACCACCGCTGGTAG |
| | CGGTTTTTTTGTTTGCAAGCAGCAGATTACGCGCAGAAAAAAAGGATCTCAAGAA |
| | GATCCTTTGATCTTTTCTACGGGGTCTGACGCTCAGTGGAACGAAAACTCACGTTA |
| | AGGGATTTTGGTCATGAGATTATCAAAAAGGATCTTCACCTAGATCCTTTTAAATTA |
| | AAAATGAAGTTTTAAATCAATCTAAAGTATATATGAGTAAACTTGGTCTGACAGTTA |
| | CCAATGCTTAATCAGTGAGGCACCTATCTCAGCGATCTGTCTATTTCGTTCATCCAT |
| | AGTTGCCTGACTCCCCGTCGTGTAGATAACTACGATACGGGAGGGCTTACCATCTG |
| | GCCCCAGTGCTGCAATGATACCGCGAGACCCACGCTCACCGGCTCCAGATTTATCA |
| | GCAATAAACCAGCCAGCCGGAAGGGCCGAGCGCAGAAGTGGTCCTGCAACTTTA |
| | TCCGCCTCCATCCAGTCTATTAATTGTTGCCGGGAAGCTAGAGTAAGTAGTTCGCC |
| | AGTTAATAGTTTGCGCAACGTTGTTGCCATTGCTACAGGCATCGTGGTGTCACGCT |
| | CGTCGTTTGGTATGGCTTCATTCAGCTCCGGTTCCCAACGATCAAGGCGAGTTACA |
| | TGATCCCCCATGTTGTGCAAAAAAGCGGTTAGCTCCTTCGGTCCTCCGATCGTTGT |
| | CAGAAGTAAGTTGGCCGCAGTGTTATCACTCATGGTTATGGCAGCACTGCATAATT |
| | CTCTTACTGTCATGCCATCCGTAAGATGCTTTTCTGTGACTGGTGAGTACTCAACC |
| | AAGTCATTCTGAGAATAGTGTATGCGGCGACCGAGTTGCTCTTGCCCGGCGTCAAT |
| | ACGGGATAATACCGCGCCACATAGCAGAACTTTAAAAGTGCTCATCATTGGAAAA |
| | CGTTCTTCGGGGCGAAAACTCTCAAGGATCTTACCGCTGTTGAGATCCAGTTCGAT |
| | GTAACCCACTCGTGCACCCAACTGATCTTCAGCATCTTTTACTTTCACCAGCGTTT |
| | CTGGGTGAGCAAAAACAGGAAGGCAAAATGCCGCAAAAAAGGGAATAAGGGCG |
| | ACACGGAAATGTTGAATACTCATACTCTTCCTTTTTCAATATTATTGAAGCATTTATC |
| | AGGGTTATTGTCTCATGAGCGGATACATATTTGAATGTATTTAGAAAAATAAACAAA |
| | TAGGGGTTCCGCGCACATTTCCCCGAAAAGTGCCACCTGACGTC |
| SEQ ID NO: 27 | GACGGATCGGGAGATCTCCCGATCCCCTATGGTGCACTCTCAGTACAATCTGCTCT |
| pcDNA2 TADA | GATGCCGCATAGTTAAGCCAGTATCTGCTCCCTGCTTGTGTGTTGGAGGTCGCTGA |
| (Adalimumab), | GTAGTGCGCGAGCAAAATTTAAGCTACAACAAGGCAAGGCTTGACCGACAATTGC |
| nt | ATGAAGAATCTGCTTAGGGTTAGGCGTTTTGCGCTGCTTCGCGATGTACGGGCCAG |
| | ATATACGCGTTGACATTGATTATTGACTAGTTATTAATAGTAATCAATTACGGGGTCA |
| | TTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCC |
| | CATAGTAACGCCAATAGGGACTTTCCATTGACGTCAATGGGTGGAGTATTTACGGT |
| | AAACTGCCCACTTGGCAGTACATCAAGTGTATCATATGCCAAGTACGCCCCCTATT |
| | GACGTCAATGACGGTAAATGGCCCGCCTGGCATTATGCCCAGTACATGACCTTATG |
| | GGACTTTCCTACTTGGCAGTACATCTACGTATTAGTCATCGCTATTACCATGGTGAT |
| | GCGGTTTTGGCAGTACATCAATGGGCGTGGATAGCGGTTTGACTCACGGGGATTTC |
| | CAAGTCTCCACCCCATTGACGTCAATGGGAGTTTGTTTTGGCACCAAAATCAACG |
| | GGACTTTCCAAAATGTCGTAACAACTCCGCCCCATTGACGCAAATGGGCGGTAGG |
| | CGTGTACGGTGGGAGGTCTATATAAGCAGAGCTCTCTGGCTAACTAGAGAACCCA |
| | CTGCTTACTGGCTTATCGAAATTAATACGACTCACTATAGGGAGACCCAAGCTAAG |
| | CTTGCCGCCACCATGAGGGTCCCCGCTCAGCTCCTGGGGCTCCTGCTGCTCTGGC |
| | TCCCAGGTGCACGATGTGATATCCAAATGACTCAAAGTCCAAGTAGTCTGTCCGCA |
| | AGCGTCGGCGATCGCGTGACCATCACATGTAGAGCTTCTCAAGGCATCCGGAACT |
| | ACCTGGCTTGGTACCAGCAGAAGCCTGGCAAGGCCCCCAAGCTGCTGATCTATGC |
| | CGCTTCTACCCTCCAGTCCGGCGTGCCTAGCAGATTTTCCGGCTCCGGATCTGGAA |
| | CAGACTTCACCCTGACCATCTCCTCTCTGCAGCCTGAGGACGTGGCCACCTACTAC |
| | TGCCAGCGGTACAACAGAGCCCCATACACCTTCGGCCAGGGCACCAAGGTGGAA |
| | ATCAAACGTACGGTGGCTGCACCATCTGTCTTCATCTTCCCGCCATCTGATGAGCA |
| | GTTGAAATCTGGAACTGCCTCTGTTGTGTGCCTGCTGAATAACTTCTATCCCAGAG |
| | AGGCCAAAGTACAGTGGAAGGTGGATAACGCCCTCCAATCGGGTAACTCCCAGGA |
| | GAGTGTCACAGAGCAGGACAGCAAGGACAGCACCTACAGCCTCAGCAGCACCCT |
| | GACGCTGAGCAAAGCAGACTACGAGAAACACAAAGTCTACGCCTGCGAAGTCAC |
| | CCATCAGGGCCTGAGCTCGCCCGTCACAAAGAGCTTCAACAGGGGAGAGTGTTG |
| | AGGATCCACTAGTCCAGTGTGGTGGAATTCAGATCCGTTAACGGTTACCAACTACC |
| | TAGACTGGATTCGTGACAACATGCGGCCGTGATATCTACGTATGATCAGCCTCGAC |
| | TGTGCCTTCTAGTTGCCAGCCATCTGTTGTTTGCCCCTCCCCCGTGCCTTCCTTGAC |
| | CCTGGAAGGTGCCACTCCCACTGTCCTTTCCTAATAAAATGAGGAAATTGCATCGC |
| | ATTGTCTGAGTAGGTGTCATTCTATTCTGGGGGGTGGGGTGGGGCAGGACAGCAA |
| | GGGGGAGGATTGGGAAGACAATAGCAGGCATGCTGGGGATGCGGTGGGCTCTATG |
| | GAACCAGCTGGGGCTCGACAGCTATGCCAAGTACGCCCCCTATTGACGTCAATGA |
| | CGGTAAATGGCCCGCCTGGCATTATGCCCAGTACATGACCTTATGGGACTTTCCTA |
| | CTTGGCAGTACATCTACGTATTAGTCATCGCTATTACCATGGTGATGCGGTTTTGGC |
| | AGTACATCAATGGGCGTGGATAGCGGTTTGACTCACGGGGATTTCCAAGTCTCCAC |
| | CCCATTGACGTCAATGGGAGTTTGTTTTGGCACCAAAATCAACGGGACTTTCCAA |
| | AATGTCGTAACAACTCCGCCCCATTGACGCAAATGGGCGGTAGGCGTGTACGGTG |
| | GGAGGTCTATATAAGCAGAGCTGGGTACGTCCTCACATTCAGTGATCAGCACTGAA |
| | CACAGACCCGCGGCCGCTCGAGGCCGCCACCATGGGTTGGAGCCTCATCTTGCTC |
| | TTCCTTGTCGCTGTTGCTACGCGTGTCGAAGTGCAACTAGTGGAAAGTGGTGGTG |
| | GTCTCGTGCAGCCCGGAAGGTCTCTGCGGCTGTCCTGTGCTGCTTCCGGCTTCAC |
| | CTTCGACGATTATGCCATGCACTGGGTCCGGCAAGCTCCTGGCAAGGGCCTGGAG |
| | TGGGTCTCTGCCATCACCTGGAATTCTGGCCACATCGACTACGCCGACTCCGTGGA |
| | AGGCAGATTTACCATCTCCAGAGACAACGCCAAGAACAGCCTGTACCTGCAGATG |
| | AACTCCCTGAGAGCCGAGGATACAGCCGTGTACTACTGCGCCAAAGTGTCCTACC |
| | TGTCTACCGCTTCTAGCCTGGACTACTGGGGCCAGGGCACCCTGGTGACCGTGTC |
| | CAGCGCTAGCACCAAGGGCCCATCGGTCTTCCCCCTGGCACCCTCCTCCAAGAGC |
| | ACCTCTGGGGGCACAGCGGCCCTGGGCTGCCTGGTCAAGGACTACTTCCCCGAAC |
| | CGGTGACGGTGTCGTGGAACTCAGGCGCCCTGACCAGCGGCGTGCACACCTTCC |
| | CGGCTGTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGCCCAGC |
| | AACACCAAGGTGGACAAGAAAGTAGAGCCCAAATCTTGTGACAAAACTCACACA |
| | TGCCCACCGTGCCCAGCACCTGAACTCCTGGGGGGACCGTCAGTCTTCCTCTTC |
| | CCCCCAAAACCCAAGGACACCCTCATGATCTCCCGGACCCCTGAGGTCACATGCG |
| | TGGTGGTGGACGTGAGCCACGAAGACCCTGAGGTCAAGTTCAACTGGTACGTGG |
| | ACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCATCGAGAA |
| | AACCATCTCCAAAGCCAAAGGGCAGCCCCGAGAACCACAGGTGTACACCCTGCC |
| | CCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAG |
| | AACAACTACAAGACCACGCCTCCCGTGCTGGACTCCGACGGCTCCTTCTTCCTCT |
| | ACAGCAAGCTCACCGTGGACAAGAGCAGGTGGCAGCAGGGGAACGTCTTCTCAT |
| | GCTCCGTGATGCATGAGGCTCTGCACAACCACTACACGCAGAAGAGCCTCTCCCT |
| | GTCTCCGGGTAAATGAGTTTAAACCCGCTGATCAGCCTCGACTGTGCCTTCTAGTT |
| | GCCAGCCATCTGTTGTTTGCCCCTCCCCCGTGCCTTCCTTGACCCTGGAAGGTGCC |
| | ACTCCCACTGTCCTTTCCTAATAAAATGAGGAAATTGCATCGCATTGTCTGAGTAG |
| | GTGTCATTCTATTCTGGGGGGTGGGGTGGGGCAGGACAGCAAGGGGGAGGATTG |
| | GGAAGACAATAGCAGGCATGCTGGGGATGCGGTGGGCTCTATGGCTTCTGAGGCG |
| | GAAAGAACCAGCTGGGGCTCTAGGGGGTATCCCCACGCGCCCTGTAGCGGCGCAT |
| | TAAGCGCGGCGGGTGTGGTGGTTACGCGCAGCGTGACCGCTACACTTGCCAGCGC |
| | CCTAGCGCCCGCTCCTTTCGCTTTCTTCCCTTCCTTTCTCGCCACGTTCGCCGGCTT |
| | TCCCCGTCAAGCTCTAAATCGGGGGCTCCCTTTAGGGTTCCGATTTAGTGCTTTAC |
| | GGCACCTCGACCCCAAAAAACTTGATTAGGGTGATGGTTCACGTAGTGGGCCATC |
| | GCCCTGATAGACGGTTTTTCGCCCTTTGACGTTGGAGTCCACGTTCTTTAATAGTG |
| | GACTCTTGTTCCAAACTGGAACAACACTCAACCCTATCTCGGTCTATTCTTTTGATT |
| | TATAAGGGATTTTGCCGATTTCGGCCTATTGGTTAAAAAATGAGCTGATTTAACAA |
| | AAATTTAACGCGAATTAATTCTGTGGAATGTGTGTCAGTTAGGGTGTGGAAAGTCC |
| | CCAGGCTCCCCAGCAGGCAGAAGTATGCAAAGCATGCATCTCAATTAGTCAGCAA |
| | CCAGGTGTGGAAAGTCCCCAGGCTCCCCAGCAGGCAGAAGTATGCAAAGCATGC |
| | ATCTCAATTAGTCAGCAACCATAGTCCCGCCCCTAACTCCGCCCATCCCGCCCCTA |
| | ACTCCGCCCAGTTCCGCCCATTCTCCGCCCCATGGCTGACTAATTTTTTTTATTTAT |
| | GCAGAGGCCGAGGCCGCCTCTGCCTCTGAGCTATTCCAGAAGTAGTGAGGAGGCT |
| | TTTTTGGAGGCCTAGGCTTTTGCAAAAAGCTCCCGGGAGCTTGTATATCCATTTTC |
| | GGATCTGATCAAGAGACAGGATGAGGATCGTTTCGCATGATTGAACAAGATGGAT |
| | TGCACGCAGGTTCTCCGGCCGCTTGGGTGGAGAGGCTATTCGGCTATGACTGGGC |
| | ACAACAGACAATCGGCTGCTCTGATGCCGCCGTGTTCCGGCTGTCAGCGCAGGGG |
| | CGCCCGGTTCTTTTTGTCAAGACCGACCTGTCCGGTGCCCTGAATGAACTGCAGG |
| | ACGAGGCAGCGCGGCTATCGTGGCTGGCCACGACGGGCGTTCCTTGCGCAGCTGT |
| | GCTCGACGTTGTCACTGAAGCGGGAAGGGACTGGCTGCTATTGGGCGAAGTGCC |
| | GGGGCAGGATCTCCTGTCATCTCACCTTGCTCCTGCCGAGAAAGTATCCATCATGG |
| | CTGATGCAATGCGGCGGCTGCATACGCTTGATCCGGCTACCTGCCCATTCGACCAC |
| | CAAGCGAAACATCGCATCGAGCGAGCACGTACTCGGATGGAAGCCGGTCTTGTCG |
| | ATCAGGATGATCTGGACGAAGAGCATCAGGGGCTCGCGCCAGCCGAACTGTTCGC |
| | CAGGCTCAAGGCGCGCATGCCCGACGGCGAGGATCTCGTCGTGACCCATGGCGAT |
| | GCCTGCTTGCCGAATATCATGGTGGAAAATGGCCGCTTTTCTGGATTCATCGACTG |
| | TGGCCGGCTGGGTGTGGCGGACCGCTATCAGGACATAGCGTTGGCTACCCGTGAT |
| | ATTGCTGAAGAGCTTGGCGGCGAATGGGCTGACCGCTTCCTCGTGCTTTACGGTAT |
| | CGCCGCTCCCGATTCGCAGCGCATCGCCTTCTATCGCCTTCTTGACGAGTTCTTCT |
| | GAGCGGGACTCTGGGGTTCGAAATGACCGACCAAGCGACGCCCAACCTGCCATC |
| | ACGAGATTTCGATTCCACCGCCGCCTTCTATGAAAGGTTGGGCTTCGGAATCGTTT |
| | TCCGGGACGCCGGCTGGATGATCCTCCAGCGCGGGGATCTCATGCTGGAGTTCTT |
| | CGCCCACCCCAACTTGTTTATTGCAGCTTATAATGGTTACAAATAAAGCAATAGCAT |
| | CACAAATTTCACAAATAAAGCATTTTTTTCACTGCATTCTAGTTGTGGTTTGTCCAA |
| | ACTCATCAATGTATCTTATCATGTCTGTATACCGTCGACCTCTAGCTAGAGCTTGGC |
| | GTAATCATGGTCATAGCTGTTTCCTGTGTGAAATTGTTATCCGCTCACAATTCCACA |
| | CAACATACGAGCCGGAAGCATAAAGTGTAAAGCCTGGGGTGCCTAATGAGTGAGC |
| | TAACTCACATTAATTGCGTTGCGCTCACTGCCCGCTTTCCAGTCGGGAAACCTGTC |
| | GTGCCAGCTGCATTAATGAATCGGCCAACGCGCGGGGAGAGGCGGTTTGCGTATT |
| | GGGCGCTCTTCCGCTTCCTCGCTCACTGACTCGCTGCGCTCGGTCGTTCGGCTGCG |
| | GCGAGCGGTATCAGCTCACTCAAAGGCGGTAATACGGTTATCCACAGAATCAGGG |
| | GATAACGCAGGAAAGAACATGTGAGCAAAAGGCCAGCAAAAGGCCAGGAACCG |
| | TAAAAAGGCCGCGTTGCTGGCGTTTTTCCATAGGCTCCGCCCCCCTGACGAGCATC |
| | ACAAAAATCGACGCTCAAGTCAGAGGTGGCGAAACCCGACAGGACTATAAAGAT |
| | ACCAGGCGTTTCCCCCTGGAAGCTCCCTCGTGCGCTCTCCTGTTCCGACCCTGCC |
| | GCTTACCGGATACCTGTCCGCCTTTCTCCCTTCGGGAAGCGTGGCGCTTTCTCATA |
| | GCTCACGCTGTAGGTATCTCAGTTCGGTGTAGGTCGTTCGCTCCAAGCTGGGCTGT |
| | GTGCACGAACCCCCCGTTCAGCCCGACCGCTGCGCCTTATCCGGTAACTATCGTCT |
| | TGAGTCCAACCCGGTAAGACACGACTTATCGCCACTGGCAGCAGCCACTGGTAAC |
| | AGGATTAGCAGAGCGAGGTATGTAGGCGGTGCTACAGAGTTCTTGAAGTGGTGGC |
| | CTAACTACGGCTACACTAGAAGAACAGTATTTGGTATCTGCGCTCTGCTGAAGCCA |
| | GTTACCTTCGGAAAAAGAGTTGGTAGCTCTTGATCCGGCAAACAAACCACCGCTG |
| | GTAGCGGTTTTTTTGTTTGCAAGCAGCAGATTACGCGCAGAAAAAAAGGATCTCA |
| | AGAAGATCCTTTGATCTTTTCTACGGGGTCTGACGCTCAGTGGAACGAAAACTCA |
| | CGTTAAGGGATTTTGGTCATGAGATTATCAAAAAGGATCTTCACCTAGATCCTTTTA |
| | AATTAAAAATGAAGTTTTAAATCAATCTAAAGTATATATGAGTAAACTTGGTCTGAC |
| | AGTTACCAATGCTTAATCAGTGAGGCACCTATCTCAGCGATCTGTCTATTTCGTTCA |
| | TCCATAGTTGCCTGACTCCCCGTCGTGTAGATAACTACGATACGGGAGGGCTTACC |
| | ATCTGGCCCCAGTGCTGCAATGATACCGCGAGACCCACGCTCACCGGCTCCAGAT |
| | TTATCAGCAATAAACCAGCCAGCCGGAAGGGCCGAGCGCAGAAGTGGTCCTGCA |
| | ACTTTATCCGCCTCCATCCAGTCTATTAATTGTTGCCGGGAAGCTAGAGTAAGTAGT |
| | TCGCCAGTTAATAGTTTGCGCAACGTTGTTGCCATTGCTACAGGCATCGTGGTGTC |
| | ACGCTCGTCGTTTGGTATGGCTTCATTCAGCTCCGGTTCCCAACGATCAAGGCGAG |
| | TTACATGATCCCCCATGTTGTGCAAAAAAGCGGTTAGCTCCTTCGGTCCTCCGATC |
| | GTTGTCAGAAGTAAGTTGGCCGCAGTGTTATCACTCATGGTTATGGCAGCACTGCA |
| | TAATTCTCTTACTGTCATGCCATCCGTAAGATGCTTTTCTGTGACTGGTGAGTACTC |
| | AACCAAGTCATTCTGAGAATAGTGTATGCGGCGACCGAGTTGCTCTTGCCCGGCG |
| | TCAATACGGGATAATACCGCGCCACATAGCAGAACTTTAAAAGTGCTCATCATTGG |
| | AAAACGTTCTTCGGGGCGAAAACTCTCAAGGATCTTACCGCTGTTGAGATCCAGT |
| | TCGATGTAACCCACTCGTGCACCCAACTGATCTTCAGCATCTTTTACTTTCACCAG |
| | CGTTTCTGGGTGAGCAAAAACAGGAAGGCAAAATGCCGCAAAAAAGGGAATAAG |
| | GGCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATAA |
| | ACAAATAGGGGTTCCGCGCACATTTCCCCGAAAAGTGCCACCTGACGTC |
| SEQ ID NO: 28 | DxExNPGP |

1. A CHO cell for expressing a sialylated glycoprotein, wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are expressed in a single transcript; wherein the exogenous sialyltransferase catalytic peptide comprises SEQ ID NO: 02, and the exogenous galactosyltransferase catalytic peptide comprises SEQ ID NO: 05.

2. (canceled)

3. The CHO cell of claim 1, comprising a first nucleic acid encoding the exogenous sialyltransferase catalytic peptide and a second nucleic acid encoding the exogenous galactosyltransferase catalytic peptide, wherein the first nucleic acid and the second nucleic acid are transcriptionally controlled by the same promoter.

4. The CHO cell of claim 3, wherein the first nucleic acid and the second nucleic acid are connected to each other via a connecting nucleic acid, which is configured to encode a ribosomal shifting peptide.

5-6. (canceled)

7. The CHO cell of claim 3, wherein the first nucleic acid and the second nucleic acid configured such that the exogenous sialyltransferase catalytic peptide and the exogenous galactosyltransferase catalytic peptide are expressed as a fusion protein.

8. (canceled)

9. The CHO cell of claim 3, wherein the first nucleic acid comprises SEQ ID NO: 11.

10-11. (canceled)

12. The CHO cell of claim 3, wherein the second nucleic acid comprises SEQ ID NO: 14.

13. (canceled)

14. The CHO cell of claim 3, wherein the promoter is a constitutive promoter or an activable promoter.

15-16. (canceled)

17. (canceled)

18-20. (canceled)

21. (canceled)

22-23. (canceled)

24. The CHO cell of claim 1, wherein the cell is deficient in fucosyltransferase 8 activity.

25-27. (canceled)

28. The CHO cell of claim 1, wherein the cell further comprises a payload nucleic acid encoding a recombinant glycoprotein, and the expression of the payload nucleic acid is transcriptionally controlled by a constitutive or an activable promoter.

29-30. (canceled)

31. A method for glycoengineering a recombinant glycoprotein comprising:
delivering an expression vector into a CHO cell according to claim 1, wherein the expression vector comprises a payload nucleic acid configured to encode the recombinant glycoprotein; and
expressing the payload nucleic acid in the cell, thereby obtaining a plurality of recombinant glycoproteins, where at least one recombinant glycoprotein of the plurality is conjugated with a sialylated glycan.

32. The method of claim 31, wherein the sialylated glycan is an α2-6 sialyl complex type (SCT) glycan.

33-35. (canceled)

36. The method of claim 31, wherein at least 50% of the plurality of the recombinant glycoproteins is conjugated with the sialylated glycan.

37. (canceled)

38. The method of claim 31, further comprising harvesting the plurality of recombinant glycoproteins within 200 hours from the expression of the payload nucleic acid in the cell.

39. (canceled)

40. The method of claim 31, wherein the recombinant glycoprotein is an antibody or an antigen-binding fragment thereof.

41-60. (canceled)

61. A cell for expressing a GlcNAc glycoprotein, being deficient in N-acetylglucosaminyltransferase I (GnTI) activity and constitutively or controllably expressing an exogenous endoglycosidase.

62-71. (canceled)

72. A method for glycoengineering a recombinant glycoprotein, comprising:
delivering an expression vector into a cell according to claim 61, wherein the expression vector comprises a payload nucleic acid configured to encode a recombinant glycoprotein; and
expressing the payload nucleic acid in the cell, thereby obtaining a plurality of recombinant glycoproteins, where at least one recombinant glycoprotein of the plurality is conjugated with a GlcNAc glycan.

73-89. (canceled)

90. A plurality of enriched recombinant glycoproteins, wherein at least 50% of the plurality of recombinant glycoproteins is configured with a GlcNAc glycan.

91-101. (canceled)

102. The CHO cell of claim 1, wherein the exogenous sialyltransferase catalytic peptide comprises SEQ ID NO: 03 or SEQ ID NO: 04.

103. The CHO cell of claim 3, wherein the first nucleic acid comprises SEQ ID NO: 12 or SEQ ID NO: 13.
